(19) United States
(12) Patent Application Publication
(10) Pub. No.: US 2013/0035358 A1
(43) Pub. Date: Feb. 7, 2013

(54) Bis Aromatic Compounds for Use as LTC4 Synthase Inhibitors

(71) Applicants: Peter Nilsson, Uppsala (SE); Benjamin Pelcman, Uppsala (SE); Martins Katkevics, Riga (LV); Robert Ronn, Uppsala (SE); Christian Krog-Jensen, Uppsala (SE)

(72) Inventors: Peter Nilsson, Uppsala (SE); Benjamin Pelcman, Uppsala (SE); Martins Katkevics, Riga (LV); Robert Ronn, Uppsala (SE); Christian Krog-Jensen, Uppsala (SE)

(21) Appl. No.: 13/634,221

(22) PCT Filed: Mar. 14, 2011
(86) PCT No.: PCT/GB2011/000358

§ 371 Date: Oct. 9, 2012

(30) Foreign Application Priority Data
Mar. 12, 2010 (GB) PCT/GB2010/000438

(51) Int. Cl. A61K 31/44; A61P 29/00; A61P 11/00; A61P 11/06; A61P 37/08; A61P 27/02; A61P 17/00; A61P 1/00; A61P 9/00; A61P 25/00; A61P 31/10; A61P 31/04; A61P 31/12; C07D 409/10; A61K 31/4436; C07D 405/10; A61K 31/443; A61K 31/4433; C07D 213/74

(52) U.S. Cl. 514/338; 546/310; 514/352; 546/281.4; 514/336; 546/283.7; 546/284.7; 546/282.4

(57) ABSTRACT

There is provided compounds of formula I,

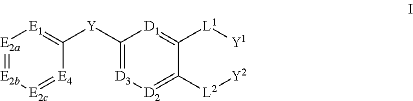

wherein E₁, E₂ₐ, E_{2b}, E_{2c}, E₄, D₁, D₂, D₃, L¹, Y¹, L² and Y² have meanings given in the description, and pharmaceutically-acceptable salts thereof, which compounds are useful in the treatment of diseases in which inhibition of leukotriene C₄ synthase is desired and/or required, and particularly in the treatment of a respiratory disorder and/or inflammation.

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. They may be converted, e.g. in the event of inflammation, into mediators, some of which are known to have beneficial properties and others that are harmful. Such mediators include leukotrienes (formed by the action of 5-lipoxygenase (5-LO), which acts by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRas) have been developed for the treatment of asthma, but they are often highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén J. Internal Med. 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRas have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of an apparently prior-published document in this specification should not necessarily be taken as an acknowledgement that the document is part of the state of the art or is common general knowledge.

International patent application WO 2008/107661 discloses various biphenyl/diphenyl compounds that may be useful as LTC₄ synthase inhibitors, and of use therefore in the treatment of inflammation. However, the two phenyl rings are linked together with via a methylene group. Further, international patent application WO 2009/030887 discloses, for that same use, various biaryl compounds linked together with a carbonyl group (i.e. diarylketones). However, there is no specific disclosure in that application of a biaryl/diaryl compound in which one of the requisite aromatic rings is a heteroaryl group.

International patent application WO 2010/103278 also discloses diarylketones, for use in the treatment of inflammation.

### DISCLOSURE OF THE INVENTION

According to the invention, there is provided a compound of formula I,

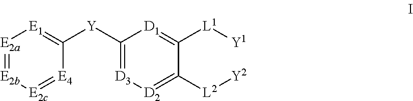

wherein
one of E₂ₐ, E_{2b} and E_{2c} represents —C(-L³-Y³)═ and the other two respectively represent E₂ and E₃;
Y represents —C(O)— or —C(═N—OR²⁸)—;
R²⁸ represents hydrogen or C₁₋₆ alkyl optionally substituted by one or more fluoro atoms;
at least one or two of D₁, D₂ and D₃ represent(s) —N═; and/or
at least one or two of E₁, E₂, E₃ and E₄ represent(s) —N═; and
those (or the) remaining D₁, D₂ and D₃ group(s) each independently represent —C(R¹)═; and
those remaining E₁, E₂, E₃ and E₄ groups each independently represent —C(R²)═;
each R¹ independently represents, on each occasion when used herein, hydrogen or a substituent selected from X¹;
each R² independently represents, on each occasion when used herein, hydrogen or a substituent selected from X²;
Y¹ represents —C(O)OR^{9a} or 5-tetrazolyl;
R^{9a} represents:
(i) hydrogen; or
(ii) C₁₋₈ alkyl or a heterocycloalkyl group, both of which are optionally substituted by one or more substituents selected from G¹ and/or Z¹;
one of Y² and Y³ represents an aryl group or a heteroaryl group (both of which groups are optionally substituted by one or more substituents selected from A) and the other represents either:
(a) an aryl group or a heteroaryl group (both of which groups are optionally substituted by one or more substituents selected from A); or
(b) C₁₋₁₂ alkyl or a heterocycloalkyl group, both of which are optionally substituted by one or more substituents selected from G¹ and/or Z¹;
A represents, on each occasion when used herein:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
X¹, X², G¹ and B independently represent halo, —R^{5a}, —C(O)R^{5b}, —CN, —NO₂, —C(O)N(R^{6a})R^{7a}, —N(R^{6b})R^{7b}, —N(R^{5c})C(O)R^{6c}, —N(R^{5d})C(O)OR^{6d}, —OR^{5e}, —OS(O)₂R^{5f}, —S(O)ₘR^{5g}, —OC(O)R^{5h} or —S(O)₂N(R^{6e})R^{7e};
R^{5b} to R^{5e}, R^{5g}, R^{5h}, R^{6a} to R^{6c}, R^{6e}, R^{7a}, R^{7b} and R^{7e} independently represent, on each occasion when used herein, H or R^{5a}; or
any of the pairs R^{6a} and R^{7a}, R^{6b} and R^{7b}, or R^{6e} and R^{7e} may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from fluoro, ═O, —OR^{5e} and/or R^{5a};
R^{5f} and R^{6d} independently represent R^{5a};
R^{5a} represents, on each occasion when used herein:
(i) C₁₋₆ alkyl optionally substituted by one or more substituents selected from fluoro, —CN, ═O, —OR^{8a}, —N(R^{8b})R^{8c}, —S(O)ₙR^{8d} and/or —S(O)₂N(R^{8e})R^{8f}; or
(ii) aryl or heteroaryl, both of which are optionally substituted by one or more substituents selected from halo, —CN, —OR^{8a}, —N(R^{8b})R^{8c}, —S(O)ₙR^{8d} and/or —S(O)₂N(R^{8e})R^{8f};
n represents 0, 1 or 2;
each R^{8b}, R^{8d} and R^{8e} independently represent H or C₁₋₆ alkyl optionally substituted by one or more substituents selected from fluoro, ═O, —OR^{11a} and/or —N(R^{12a})R^{12b};
each R^{8a}, R^{8c} and R^{8f} independently represent H or C₁₋₃ alkyl optionally substituted by one or more substituents selected from F, ═O, —OR^{13a}, —N(R^{14a})R^{14b}, —S(O)₂CH₃, —S(O)₂CHF₂ and/or —S(O)₂CF₃; or
R^{8b} and R^{8c} and/or R^{8e} and R^{8f} may be linked together to form, along with the atom(s) to which they are attached, a 3- to 6-membered ring, optionally substituted by one or more substituents selected from fluoro and C₁₋₂ alkyl;
R^{11a} and R^{13a} independently represent H or C₁₋₃ alkyl optionally substituted by one or more fluoro atoms;
R^{12a}, R^{12b}, R^{14a} and R^{14b} independently represent H, —CH₃ or —CH₂CH₃;
Z¹ represents, on each occasion when used herein, ═O or ═NOR^{16b};
R^{16b} represents hydrogen or C₁₋₆ alkyl optionally substituted by one or more fluoro atoms;
L¹ represents a single bond or —(CH₂)ₚ-Q-(CH₂)_{q}—;
Q represents —C(R^{y1})(R^{y2})—, —C(O)—, —N(R^{y3})— or —O—;
p and q independently represent 0, 1 or 2, but wherein the sum of p and q does not exceed 2;
one of L² and L³ represents —C(O)-A¹⁷- (e.g. —C(O)—) and the other may represent a single bond or a spacer group selected from —S(O)ₙ₁—, —C(R^{y4})(R^{y5})-A¹⁶, —N(R^{17a})-A¹⁶-, —OA¹⁷- and —C(O)-A¹⁷- (e.g. —C(O)—);
n1 represents 0, 1 or 2;
A¹⁶ represents a direct (i.e. a single) bond, —C(R^{y6})(R^{y7})—, —C(O)—, —C(O)N(R^{17b})—, —C(O)C(R^{y6})(R^{y7})— or —S(O)₂—;
each A¹⁷ independently represents a direct bond or —C(R^{y8})(R^{y9})—;
each R^{y1}, R^{y2}, R^{y4}, R^{y5}, R^{y6}, R^{y7}, R^{y8} and R^{y9} independently represent H, fluoro or C₁₋₃ alkyl optionally substituted by one or more fluoro atoms; or
R^{y1} and R^{y2}, R^{y4} and R^{y5}, R^{y6} and R^{y7} and R^{y8} and R^{y9} may be linked together to form a 3- to 6-membered ring optionally substituted by one or more substituents selected from fluoro and C₁₋₂ alkyl;
R^{y3} represents hydrogen or C₁₋₃ alkyl;
R^{17a} and R^{17b} independently represent hydrogen, C₁₋₆ alkyl (optionally substituted by one or more substituents selected from heterocycloalkyl, aryl, heteroaryl (which latter two groups are optionally substituted by one or more substituents selected from R³⁰), fluoro, —CN, —OR¹⁹ and/or ═O), aryl or heteroaryl (both of which latter two groups are optionally substituted by one or more substituents selected from R³¹);
R³⁰ and R³¹ independently represent halo, —R^{18a}, —C(O)R^{18b}, —CN, —C(O)N(R^{18c})R^{18d}, —N(R^{18e})R^{18f}, —N(R^{18g})C(O)R^{18h}, —N(R¹⁸ⁱ)C(O)OR^{18j}, —OR^{18k}, —OS(O)₂R^{18m}, —S(O)ₘR¹⁸ⁿ, —OC(O)R^{18p} or —S(O)₂N(R^{18q})R^{18r});
m represents, on each occasion when used herein, 0, 1 or 2;
R^{18a}, R^{18b}, R^{18c}, R^{18d}, R^{18e}, R^{18f}, R^{18g}, R^{18h}, R¹⁸ⁱ, R^{18k}, R¹⁸ⁿ, R^{18p}, R^{18q} and R^{18r} independently represent hydrogen or C₁₋₃ alkyl optionally substituted by one or more fluoro atoms;
R^{18j} and R^{18m} independently represent C₁₋₃ alkyl optionally substituted by one or more fluoro atoms;
R¹⁹ represents hydrogen or C₁₋₆ alkyl optionally substituted by one or more fluoro atoms;
or a pharmaceutically-acceptable salt thereof,
which compounds and salts are referred to hereinafter as “the compounds of the invention”. Such compounds are characterised in that at least one of D₁, D₂, D₃, E₁, E₂, E₃ and E₄ represents —N═. That is, either one of (or both) the D₁ to D₃-containing ring and the E₁ to E₄-containing ring contains (at least one) —N═.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group). Where the number of carbon atoms permits, C_{1-q} alkyl groups may also be spiro-groups (i.e. two cycloalkyl rings linked together by a single common carbon atom), although they are preferably not so.

The term “halo”, when used herein, includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten and, most preferably, between three and eight, e.g. a 5- or 6-membered heterocycloalkyl group). Further, such heterocycloalkyl groups may be saturated or unsaturated containing one or more double and/or triple bonds, forming for example a C_{2-q} (e.g. C_{4-q}) heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. At each occurrence when mentioned herein, a heterocycloalkyl group is preferably a 3- to 8-membered heterocycloalkyl group (e.g. a 5- or 6-membered heterocycloalkyl group).

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are preferably linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl) and, more preferably, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazopyridyl (such as imidazo[4,5-b]pyridyl, imidazo[5,4-b]pyridyl and, preferably, imidazo[1,2-a]pyridyl), indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are polycyclic, they are preferably linked to the rest of the molecule via an aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which X¹ and X² both represent R^{5a}, i.e. a C₁₋₆ alkyl group optionally substituted as hereinbefore defined, the alkyl groups in question may be the same or different. Similarly, when groups are substituted by more than one substituent as defined herein, the identities of those individual substituents are not to be regarded as being interdependent. For example, when there are two X¹ substituents present, which represent —R^{5a} and —C(O)R^{5b} in which R^{5b} represents R^{5a}, then the identities of the two R^{5a} groups are not to be regarded as being interdependent. Likewise, when Y² or Y³ are substituted by more than one G¹ group, then such substituents are not interdependent (i.e. they may be the same or different G¹ groups). For example, when Y² or Y³ represent e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R^{5a} to R^{5h}” is employed herein, this will be understood by the skilled person to mean R^{5a}, R^{5b}, R^{5c}, R^{5d}, R^{5e}, R^{5f}, R^{5g} and R^{5h} inclusively.

For the avoidance of doubt, when the term “an R⁵ group” is referred to herein, we mean any one of R^{5a} to R^{5h}. For the avoidance of doubt, the term “E₁ to E₄-containing ring” refers to the ring containing E₁, E₂ₐ, E_{2b}, E_{2c} and E₄. Further, the term “D₁ to D₃-containing ring” refers to the ring containing D₁, D₂ and D₃.

For the avoidance of doubt, the following compounds of formula Ia, Ib and Ic are included within the scope of the compounds of formula I:

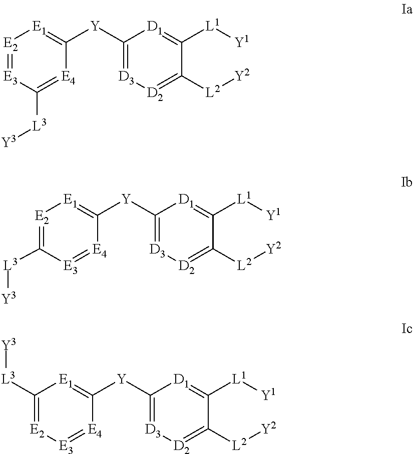

wherein the integers are as defined above. The skilled person will further appreciate that compounds of formula Ia and Ic may be identical, due to rotation around the bond linking the Y group to the E₁ to E₄-containing ring or to the D₁ to D₃-containing ring. Hence, the skilled person will appreciate that, given that there is an essential ‘-L³-Y³’ group present in the compound of formula I, then one -L³-Y³ group, as it is an essential feature.

Compounds of the invention, for instance those compounds of formula I in which L² represents —C(O)— (and Y² is as defined herein), L¹ represents a single bond and Y¹ represents —C(O)OR^{9a} (and R^{9a} is preferably hydrogen), may exist in cyclised form and/or in equilibrium with a corresponding compound in cyclised form. By cyclised form, we mean a form in which two substituents of the same molecule undergo an intramolecular cyclisation (e.g. a reversible intramolecular cyclisation), including the following compounds of formula IA,

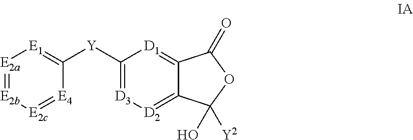

in which the integers are as defined herein (i.e. in respect of compounds of formula I and other preferred compounds of the invention). Such compounds may exist in particular when Y² represents C₁₋₁₂ alkyl as hereinbefore defined (e.g. acyclic C₁₋₁₂ alkyl). Such compounds are encompassed within the scope of compounds of the invention (and fall within the scope of compounds of formula I). Hence, compounds of formula I in which Y² represents —C(O)— may exist as such, may exist as compounds of formula IA, or may exist as a mixture of both (i.e. the compounds may be in equilibrium, such as a slow or rapid equilibrium measured on an NMR time scale). In such instances, the exact amount of compound of formula I or compound of formula IA may depend on the acidity of the environment, the solvent, concentration, temperature, and other factors known to the skilled person. In a further embodiment of the invention, there is provided a compound of formula IA as such and as defined above (which would include corresponding compounds of formula I in which Y² represents —C(O)—).

Compounds of the invention that may be mentioned include those in which:

n1 represents 1;
A¹⁶ represents a direct bond, —C(O)—, —C(O)N(R^{17b})—, —C(O)C(R^{y6})(R^{y7})— or —S(O)₂—;
R^{5a} represents, on each occasion when used herein, C₁₋₆ alkyl optionally substituted by one or more substituents selected from fluoro, —CN, ═O, —OR^{8a}, —N(R^{8b})R^{8c}, —S(O)ₙR^{8d} and/or —S(O)₂N(R^{8e})R^{8f};
R^{17a} and R^{17b} independently represent hydrogen, C₁₋₆ alkyl (optionally substituted by one or more substituents selected from fluoro, —CN, —OR¹⁹ and/or ═O), aryl or heteroaryl (both of which latter two groups are optionally substituted by one or more substituents selected from halo, —R^{18a}, —C(O)R^{18b}, —CN, —C(O)N(R^{18c})R^{18d}, —N(R^{18e})R^{18f}, —N(R^{18g})C(O)R^{18h}, —N(R¹⁸ⁱ)C(O)OR^{18j}, —OR^{18k}, —OS(O)₂R^{18m}, —S(O)ₘR¹⁸ⁿ, —OC(O)R^{18p} or —S(O)₂N(R^{18q})R^{18r});
X¹, X², G¹ and B independently represent halo, —R^{5a}, —C(O)R^{5b}, —CN, —C(O)N(R^{6a})R^{7a}, —N(R^{6b})R^{7b}, —N(R^{5c})C(O)R^{6c}, —N(R^{5d})C(O)OR^{6d}, —OR^{5e}, —OS(O)₂R^{5f}, —S(O)ₘR^{5g}, —OC(O)R^{5h} or —S(O)₂N(R^{6e})R^{7e};
each R^{8a}, R^{8b}, R^{8d} and R^{8e} independently represent H or C₁₋₆ alkyl optionally substituted by one or more substituents selected from fluoro, ═O, —OR^{11a} and/or —N(R^{12a})R^{12b};
when L² or L³ represent C(R^{y4})(R^{y5})-A¹⁶ in which A¹⁶ is other than a direct/single bond, then A¹⁶ is preferably —C(O)—.

Further compounds of the invention that may be mentioned include those in which:

when D₁, D₂, D₃, E₁, E₂, E₃ and E₄ represents —C(R¹)═ or —C(R²)═ (as appropriate), in which R¹ or R² represent a substituent defined by R^{5a}, then R^{5a} preferably represents C₁₋₆ alkyl optionally substituted as defined herein;
when L² or L³ (especially L²) represents a single bond, then Y² preferably does not represent a 5-membered heteroaryl group, an ortho-substituted phenyl group (in which the ortho substituent is e.g. an aromatic group, alkyl or heterocycloalkyl moiety, especially an aromatic group), naphthyl, a 9- or 10-membered heteroaryl group, a cycloalkyl group or a vinyl moiety (e.g. a bicyclic 5,6-fused heteroaryl group linked via the 5-membered ring; a 5-membered heteroaryl group substituted with at least one aromatic, alkyl or heterocycloalkyl (e.g. aromatic) group; a phenyl group substituted at the ortho-position e.g. with an aromatic group; or a vinylic moiety terminally substituted with e.g. an aromatic group).

Further compounds of the invention that may be mentioned include those in which:

L² represents a single bond or a spacer group selected from —C(R^{y4})(R^{y5})—, —N(R^{17a})-A¹⁶- and —OA¹⁷- (in which case L³ must represent —C(O)-A¹⁷-);
L² represents a spacer group selected from —S(O)—, —C(R^{y4})(R^{y5})—, —N(R^{17a})-A¹⁶- and —OA¹⁷-(in which case L³ must represent —C(O)-A¹⁷-);
L² represent a spacer group selected from —C(R^{y4})(R^{y5})—, —N(R^{17a})-A¹⁶- and —OA¹⁷- (in which case L³ must represent —C(O)-A¹⁷-).

Compounds of the invention that may be mentioned include those in which:

when R^{5a} represents C₁₋₆ alkyl, then that alkyl group may not be substituted at a terminal position of the alkyl group by both ═O and —OR^{8a} (hence, when R^{5a} represents C₁₋₆ alkyl, then it may not be substituted by a —C(O)OR^{8a} group);
when R^{5a} represents C₁₋₆ alkyl, then that alkyl group may not be substituted at a terminal position of the alkyl group by both ═O and —N(R^{8b})R^{8c} (hence, when R^{5a} represents C₁₋₆ alkyl, then it may not be substituted by a —C(O)N(R^{8b})R^{8c} group); when any of R^{8a}, R^{8b}, R^{8d} and R^{8e} represent C₁₋₆ alkyl, then that alkyl group may not be substituted at a terminal position of the alkyl group by both ═O and —OR^{11a} (hence, when such groups represent C₁₋₆ alkyl, then it may not be substituted by a —C(O)OR^{11a} group);
when any of R^{8a}, R^{8b}, R^{8d} and R^{8e} represent C₁₋₆ alkyl, then that alkyl group may not be substituted at a terminal position of the alkyl group by both ═O and —N(R^{12a})R^{12b} (hence, when such groups represent C₁₋₆ alkyl, then it may not be substituted by a —C(O)N(R^{12a})R^{12b} group);
when any of R^{8c} and/or R^{8f} represent C₁₋₃ alkyl, then that alkyl group may not be substituted at a terminal position of the alkyl group by both ═O and —OR^{13a} (hence, when such groups represent C₁₋₃ alkyl, then it may not be substituted by a —C(O)OR^{13a} group);
when any of R^{8c} and/or R^{8f} represent C₁₋₃ alkyl, then that alkyl group may not be substituted at a terminal position of the alkyl group by both ═O and —N(R^{14a})R^{14b} (hence, when such groups represent C₁₋₃ alkyl, then it may not be substituted by a —C(O)N(R^{14a})R^{14b} group);
when R^{17a} or R^{17b} represent a C₁₋₆ alkyl group, then that alkyl group may not be substituted at a terminal position by both a ═O and —OR¹⁹, i.e. it may not be substituted by a —COOR¹⁹ group.

Further compounds of the invention that may be mentioned include those in which:

R^{5a} represents, on each occasion when used herein, C₁₋₆ alkyl optionally substituted by one or more substituents selected from fluoro, —CN, —OR^{8a}, —N(R^{8b})R^{8c}, —S(O)ₙR^{8d} and/or —S(O)₂N(R^{8e})R^{8f}; or
R^{5a} represents, on each occasion when used herein, C₁₋₆ alkyl optionally substituted by one or more substituents selected from fluoro, —CN, ═O, —N(R^{8b})R^{8c}, —S(O)ₙR^{8d} and/or —S(O)₂N(R^{8e})R^{8f};
R^{8a}, R^{8b}, R^{8d} and R^{8e} independently represent H or C₁₋₆ alkyl optionally substituted by one or more substituents selected from fluoro, —OR^{11a} and/or —N(R^{12a})R^{12b}; or R^{8a}, R^{8b}, R^{8d} and R^{8e} independently represent H or C₁₋₆ alkyl optionally substituted by one or more substituents selected from fluoro, ═O and/or —N(R^{12a})R^{12b};
R^{8c} and R^{8f} independently represent H or C₁₋₃ alkyl optionally substituted by one or more substituents selected from F, —OR^{13a}, —N(R^{14a})R^{14b}, —S(O)₂CH₃, —S(O)₂CHF₂ and/or —S(O)₂CF₃; or
R^{8c} and R^{8f} independently represent H or C₁₋₃ alkyl optionally substituted by one or more substituents selected from F, ═O, —N(R^{14a})R^{14b}, —S(O)₂CH₃, —S(O)₂CHF₂ and/or —S(O)₂CF₃; and/or
when R^{17a} and R^{17b} represent optionally substituted C₁₋₆ alkyl, then the optional substituents are preferably selected from fluoro, —CN and/or —OR¹⁹ (or may alternatively be selected from fluoro, —CN and ═O);
when alkyl groups mentioned herein are substituted by halo, then that halo group is preferably fluoro.

In the compounds of the invention, when any of the pairs R^{6a} and R^{7a}, R^{6b} and R^{7b} and/or R^{6e} and R^{7e} are linked together to form a 3- to 6-membered ring, then preferably:

such rings are preferably 5- or 6-membered;
the ring so formed does not contain any further heteroatoms (other than the requisite nitrogen atom to which the relevant R⁶ and R⁷ groups are necessarily attached);
when such rings are substituted with R^{5a} then R^{5a} represents C₁₋₃ alkyl (e.g. ethyl, n-propyl or, more preferably, methyl) optionally substituted by one or more fluoro atoms (so forming, for example, a trifluoromethyl or difluoromethyl group);
such rings may be substituted with one or more substitutents selected from —OR^{5h} (e.g. —OH, —OCH₃, —OCF₃ or —OCHF₂) and, preferably, fluoro, ═O and, especially, R^{5a} (for example, as defined above), but are more preferably unsubstituted.

In the compounds of the invention, when any of the pairs R^{8b} and R^{8c} and/or R^{8e} and R^{8f}, are linked together to form a 3- to 6-membered ring, then preferably: such rings are preferably 5- or 6-membered;

when such rings are substituted, then they are preferably substituted with one or two substituents;
such rings are preferably unsubstituted.

In the compounds of the invention, when any of the pairs R^{y1} and R^{y2}, R^{y4} and R^{y5}, R^{y6} and R^{y7} and/or R^{y8} and R^{y9} are linked together to form a 3- to 6-membered ring, then preferably:

such rings are preferably 4-membered or, more preferably, 3-membered;
when such rings are substituted, then they are preferably substituted with one or two substituents;
such rings are preferably unsubstituted.

As stated herein, compounds of the invention that may be mentioned include those in which one or two of D₁, D₂ and D₃ represent(s) —N═ and/or one or two of E₁, E₂, E₃ and E₄ represent(s) —N═. The skilled person will appreciate that in the compounds of the invention at least one of the D₁ to D₃-containing ring and E₁ to E₄-containing ring contains (a) nitrogen atom(s) (i.e. either one of those rings, or both of those rings contains two or preferably one nitrogen atom(s)). Preferably, either one or the other of those D₁ to D₃-containing ring and E₁ to E₄-containing rings (preferably the E₁ to E₄-containing ring) contains two or preferably one nitrogen atom(s), and the other does not contain any nitrogen atoms (i.e. the relevant moieties D₁, D₂, D₃, E₁, E₂, E₃ and E₄ represent —C(R¹)═ or —C(R²)═ as appropriate).

Compounds of the invention that may be mentioned include those in which:

any one or two of E₁, E₂, E₃ and E₄ represent(s) —N═, and the others each independently represent —C(R²)═);
each of D₁, D₂ and D₃ independently represent —C(R¹)═, or each of D₁, D₂ and D₃ may alternatively and independently represent —N═.

Preferred compounds of the invention include those in which:
(i) either: at least one or two of D₁, D₂ and D₃ represent(s) —N═; or at least one or two of E₁, E₂, E₃ and E₄ represent(s) —N═ (and those remaining D₁, D₂ and D₃ groups each independently represent —C(R¹)═, and those remaining E₁, E₂, E₃ and E₄ groups each independently represent —C(R²)═);
(ii) when at least one or two of D₁, D₂ and D₃ represent(s) —N═, then E₁, E₂, E₃ and E₄ each independently represent —C(R²)═;
(iii) when at least one or two of E₁, E₂, E₃ and E₄ represent(s) —N═, then D₁, D₂ and D₃ each independently represent —C(R¹)═;
(iv) any one or two of E₁, E₂, E₃ and E₄ represent(s) —N═, and the others independently represent —C(R²)═, and each of D₁, D₂ and D₃ respectively represent —C(R¹)═;
(v) any one or two of D₁, D₂ and D₃ represent(s) —N═, and the others independently represent —C(R¹)═, and each of E₁, E₂, E₃ and E₄ respectively represent —C(R²)═.

The most preferred of the above preferences are (i) and, especially, (iv).

Preferred compounds of the invention that may be mentioned include those in which:

Y² and Y³ independently represent an aryl group or a heteroaryl group, both of which groups are optionally substituted by one or more substituents selected from A;
when Y² or Y³ represent optionally substituted C₁₋₁₂ alkyl, then it is preferably optionally substituted cycloalkyl (such as C₃₋₁₂ (e.g. C₃₋₈) cycloalkyl and, preferably, C₅₋₆ alkyl);
Y² and Y³ independently represent cyclic groups optionally substituted as defined herein, i.e. aryl, heteroaryl (which latter two groups are optionally substituted by one or more substituents selected from A), cycloalkyl or heterocycloalkyl (which latter two groups are as defined herein; and both of which are optionally substituted by one or more substituents selected from G¹ and/or Z¹);
Y represents —C(O)—.

Further preferred compounds of the invention that may be mentioned include those in which:

one of L² and L³ represent(s) a spacer group selected from —C(R^{y4})(R^{y5})—, —N(R^{17a})-A¹⁶-, and —OA¹⁷- (and the other represents the requisite —C(O)-A¹⁷-moiety);
(e.g. one of) Y² and Y³ represent an aryl group optionally substituted as defined herein;
when L² or L³ represent —N(R^{17a})A¹⁶-, in which A¹⁶ represents a single bond and R^{17a} represents H, then Y² or Y³ (as appropriate) preferably does/do not represent a benzimidazolyl (e.g. benzimidazol-2-yl) group.

Preferred rings that the D₁ to D₃-containing ring may represent include 2- or 4-pyridyl (relative to the point of attachment to the —C(O)— (or —C(═N—OR²⁸)—) moiety) or, most preferably, phenyl.

Preferred rings that the E₁ to E₄-containing ring may represent include pyrazinyl, pyrimidinyl, pyridazinyl and, preferably, pyridyl groups. For example:

when two of E₁, E₂, E₃ and E₄ represent —N═, then preferably the E₁ to E₄-containing ring represents a pyrazinyl, pyrimidinyl or pyridazinyl (e.g. E₁ and E_{2c}, E₂ₐ and E₄, E₁ and E_{2b}, E₂ₐ and E_{2c}, E_{2b} and E₄, E₁ and E₄, E₁ and E₂ₐ, E₂ₐ and E_{2b}, E_{2b} and E_{2c}, or E_{2c} and E₄ may be the two E₁ to E₄ groups that represent —N═, so forming for example, a 2-pyrazinyl, 2-pyrimidinyl, 4-pyrimidinyl, 5-pyrimidinyl, 3-pyridazinyl or 4-pyridazinyl) group (especially preferred are 2-pyrimidinyl groups);
preferably, only one of E₁, E₂ₐ, E_{2b}, E_{2c} and E₄ (e.g. E_{2b}, preferably, E₂ₐ or E_{2c}, or, especially, one of E₁ or E₄, i.e. one of the ortho positions, relative to the point of attachment with the Y moiety) represents —N═ (and the others each independently represent —C(R²)═, as appropriate), and hence the E₁ to E₄-containing ring is preferably a pyridyl (e.g. 4-pyridyl, 3-pyridyl or especially a 2-pyridyl) group.

Preferred aryl and heteroaryl groups that Y² and Y³ may independently represent include optionally substituted (i.e. by A) phenyl, naphthyl (e.g. 5,6,7,8-tetrahydronaphthyl), pyrrolyl, furanyl, thienyl (e.g. 2-thienyl or 3-thienyl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred groups include thienyl, thiazolyl, oxazolyl and phenyl.

Preferred substituents on Y² and Y³ groups include:

halo (e.g. bromo or, preferably, fluoro or chloro);
cyano;
C₁₋₆ alkyl, which alkyl group may be cyclic, part-cyclic, unsaturated or, preferably, linear or branched (e.g. C₁₋₄ alkyl (such as ethyl, n-propyl, isopropyl, t-butyl or, preferably, n-butyl or methyl), all of which are optionally substituted with one or more halo (e.g. fluoro) groups (so forming, for example, fluoromethyl, difluoromethyl or, preferably, trifluoromethyl);
heterocycloalkyl, such as a 5- or 6-membered heterocycloalkyl group, preferably containing a nitrogen atom and, optionally, a further nitrogen or oxygen atom, so forming for example morpholinyl, piperazinyl, piperidinyl or pyrrolidinyl, which heterocycloalkyl group is optionally substituted by one or more (e.g. one or two) substituents selected from C₁₋₃ alkyl (e.g. methyl) and ═O;

### —OR²⁶;

### —C(O)R²⁶;

### —C(O)OR²⁶;

### —N(R²⁶)R²⁷; and

—S(O)ₘR²⁶ (in which m is 0, 1 or 2);
wherein R²⁶ and R²⁷ independently represent, on each occasion when used herein, H, C₁₋₆ alkyl, such as C₁₋₄ alkyl (e.g. ethyl, n-propyl, t-butyl or, preferably, n-butyl, methyl or isopropyl) optionally substituted by one or more halo (e.g. fluoro) groups (so forming e.g. a perfluoroethyl or, preferably, a trifluoromethyl group) or aryl (e.g. phenyl) optionally substituted by one or more halo or C₁₋₃ (e.g. C₁₋₂) alkyl groups (which alkyl group is optionally substituted by one or more halo (e.g. fluoro) atoms). Preferably, when the substituent is —S(O)R²⁶ or —S(O)₂R²⁶, then R²⁶ does not represent hydrogen.

Preferred compounds of the invention include those in which:

any two (preferably any one or, more preferably, none) of D₁, D₂ and D₃ represents —N═;
any one of E₁, E₂, E₃ and E₄ represents —N═ and the others independently represent —C(R²)═;
E₁ (or E₄) represents —N═ or —C(R²)═;
E₂ₐ (or E_{2c}) represents —C(R²)═ or —N═;
E_{2b} represents —C(-L³-Y³)═;
when three R² groups are present, then at least one (e.g. at least two) of those R² groups represents hydrogen;
when two R² groups are present, then at least one of them represents hydrogen; at least one (e.g. at least two) R¹ group that may be present represents hydrogen;
X¹, X², G¹ and B independently represent —C(O)N(R^{6a})R^{7a}, —N(R^{6b})R^{7b} or, preferably, halo (e.g. chloro or fluoro), —R^{5a}, —OR^{5e} or —S(O)ₘR^{5g};
R^{8a}, R^{8b}, R^{8d} and R^{8e} independently represent hydrogen or C₁₋₆ (e.g. C₁₋₃) alkyl optionally substituted by one or more substituents selected from —OR^{11a}, preferably, ═O, and especially, fluoro (most preferably, R^{8a}, R^{8b}, R^{8d} and R^{8e} independently represent —CF₃, methyl or particularly, hydrogen);
R^{8c} and R^{8f} independently represent hydrogen or C₁₋₃ (e.g. C₁₋₂) alkyl optionally substituted by one or more substituents selected from —OR^{13a}, preferably, ═O, and especially, fluoro (most preferably, R^{8c} and R^{8f} independently represent —CF₃, methyl or particularly, hydrogen);
R^{11a} and R^{13a} independently represent —CF₃, preferably ethyl, and particularly hydrogen and/or methyl;
R^{12a}, R^{12b}, R^{14a} and R^{14b} independently represent methyl or hydrogen (particularly, methyl);
L¹ represents a single bond;
Y¹ represents 5-tetrazolyl (which is preferably unsubstituted) or, preferably, —C(O)OR^{9a};
R^{9a} represents C₁₋₆ alkyl (optionally substituted by one or more G¹ and/or Z¹ substituents; but preferably unsubstituted) or, preferably, hydrogen;
at least one of Y² and Y³ represents aryl (e.g. phenyl) optionally substituted as defined herein;
Y² and Y³ may be the same or different;
A represents aryl or heteroaryl (e.g. aryl, such as phenyl optionally substituted by halo, e.g. fluoro or chloro), but A preferably represents G¹ or C₁₋₆ (e.g. C₁₋₄) alkyl (e.g. butyl (such as n-butyl) or methyl) optionally substituted by one or more substituents selected from G¹;
R^{5a} represents C₁₋₆ (e.g. C₁₋₄) alkyl optionally substituted by one or more substituents selected from —N(R^{8b})R^{8c} and, preferably, fluoro and —OR^{8a};
R^{6a} and R^{7a}, R^{6b} and R^{7b} and/or R^{6e} and R^{7e} are preferably not linked together;
when R^{5e} represents R^{5a}, then R^{5a} preferably represents C₁₋₆ (e.g. C₁₋₄) alkyl (which group may be substituted by one or more fluoro atoms, but is more preferably unsubstituted);
Z¹ represents ═O;
R^{16b} represents C₁₋₂ alkyl (e.g. methyl) or, preferably, hydrogen;
L¹ represents a single bond;
Q represents —C(R^{y1})(R^{y2})—;
p and q represent 0 or 1;
the sum of p and q is 0 or 1;
R^{y1} and R^{y2} independently represent fluoro, methyl or, preferably, hydrogen;
R^{y1} and R^{y2} are preferably not linked together;
R^{y3} represents hydrogen or methyl;
one of L² and L³ represents —C(O)-A¹⁷- and the other represents a single bond or, more preferably, —N(R^{17a})-A¹⁶- or —OA¹⁷-;
A¹⁶ represents a direct bond, —C(O)— or —S(O)₂—;
R^{y4}, R^{y5}, R^{y6}, R^{y7}, R^{y8} and R^{y9} independently represent fluoro, methyl or, preferably, hydrogen;
R^{y4} and R^{y5}, R^{y6} and R^{y7} and/or R^{y8} and R^{y9} are preferably not linked together;
when R^{17a} or R^{17b} represent optionally substituted aryl or heteroaryl, then those optional substituents are preferably selected from halo (e.g. fluoro and chloro) and R^{18a};
R^{17a} and R^{17b} represents hydrogen or C₁₋₆ alkyl optionally substituted as hereinbefore defined (for example, by one or more substituents selected from fluoro, —CN, —OH, —OCH₃ and —OCH₂CH₃);
R^{18a}, R^{18b}, R^{18c}, R^{18d}, R^{18e}, R^{18f}, R^{18g}, R^{18h}, R¹⁸ⁱ, R^{18k}, R¹⁸ⁿ, R^{18p}, R^{18q} and R^{18r} independently represent —CHF₂ or, preferably, hydrogen, methyl or —CF₃;
R^{18j} and R^{18m} independently represent —CHF₂ or, preferably, methyl or —CF₃;
when Y² and/or Y³ represent an optionally substituted phenyl group, then that phenyl group may be substituted with a single substituent (e.g. at the para-, meta- or ortho-position) or with two substituents (e.g. with one at the para-position and the other at the meta-position or with one at the ortho- and the other at the meta-position, so forming for example a 3,4-substituted or 2,5-substituted phenyl group);
R²⁸ represents hydrogen or unsubstituted C₁₋₃ (e.g. C₁₋₂) alkyl (e.g. methyl).

More preferred compounds of the invention include those in which:

E_{2b} represents —C(-L³-Y³)═ (and hence, E₂ₐ and E_{2c} respectively represent E₂ and E₃);
E₁ represents —N═;
E₄ represents —N═ or, preferably, —C(R²)═;
E₂ and E₃ independently represent —C(R²)═;
each R² independently represents hydrogen;
D₂ represents —C(R¹)═;
D₁ and D₃ independently represent —C(R¹)═ or —N═;
most preferably, each D₁, D₂ and D₃ independently represents —C(R¹)═ (e.g. D₁, D₂ and D₃ independently represent —C(H)═);
each R¹ independently represents, on each occasion when used herein, hydrogen;
only one of the D₁ to D₃-containing ring and the E₁ to E₄-containing ring (preferably the E₁ to E₄-containing ring) contains a nitrogen atom (i.e. —N═) and the other (preferably the D₁ to D₃-containing ring) does not contain a nitrogen atom;
when the D₁ to D₃-containing ring contains a nitrogen atom, then preferably, either D₁ or D₃ represents —N═ and D₂ represents —C(R¹)═ (so forming, for example, a 2-pyridyl group);
when the E₁ to E₄-containing ring contains a nitrogen atom, then preferably, either E₁ or E₄ or both E₁ and E₄ represent(s) —N═ and E₂ and E₃ independently represent —C(R²)═ (so forming, for example, a 2-pyridyl group or a 2-pyrimidinyl group);
X¹, X² and B independently represent halo (e.g. chloro or fluoro), —R^{5a} or —OR^{5e} (most preferably, X¹, X² and B independently represent —R^{5a} or, preferably, halo (e.g. chloro or fluoro));
Y represents —C(O)—;
L¹ represents a single bond;
Y¹ represents —C(O)OR^{9a};
R^{9a} represents hydrogen;
one of L² and L³ represents —C(O)-A¹⁷- (e.g. —C(O)—) and the other represents a single bond, or, preferably —N(R^{17a})-A¹⁶- or —OA¹⁷-;
A¹⁶ represents a direct bond, —C(O)— or —S(O)₂—;
when L³ (or L²) represents —N(R^{17a})-A¹⁶-, then A¹⁶ preferably represents a direct bond;
A¹⁷ represents a direct bond;
R^{17a} represents hydrogen or C₁₋₆ alkyl optionally substituted by one or more (e.g. one) substituent(s) selected from —OCH₃, —OCH₂CH₃ and —CN;
when R^{17a} represents optionally substituted C₁₋₆ alkyl, then that group may represent: a linear unsaturated C₁₋₆ (e.g. C₁₋₄, such as C₁₋₃) alkyl group (e.g. methyl, ethyl or propyl) optionally substituted by —OCH₃, —OCH₂CH₃ and/or —CN, so forming for example a methoxyethyl (i.e. —(CH₂)₂—OCH₃), ethoxyethyl or cyanopropyl (i.e. —(CH₂)₃—CN); a part cyclic C₁₋₆ alkyl group (for example C₁₋₂ alkyl (e.g. methyl) substituted by C₃₋₅ cycloalkyl), such as cyclopropylmethyl (i.e. —CH₂-cyclopropyl), cyclobutylmethyl or cyclopentylmethyl; a linear saturated C₁₋₆ (e.g. C₁₋₄, such as C₁₋₃) alkyl group (in which the unsaturation is preferably one double or one triple bond), such as allyl (i.e. —CH₂—CH═CH) or propynyl (i.e. —CH₂—CH≡CH);
Y² and Y³ independently represent an aryl (e.g. phenyl) or heteroaryl (e.g. triazolyl, or, preferably, thiazolyl, oxazolyl or thienyl) group optionally substituted by one or more substitutents selected from A;
A represents aryl (optionally substituted by halo, such as chloro), or, preferably, G¹;
G¹ represents halo (e.g. chloro or fluoro), —R^{5a}, —OR^{5e} or —S(O)ₘR^{5g};
R^{5g} represents R^{5a};
R^{5a} represents C₁₋₆ (e.g. C₁₋₄) alkyl (such as methyl or butyl, e.g. n- or t-butyl; which alkyl group is optionally substituted by one or more fluoro atoms, so forming for example, a —CF₃ group);
when R^{5e} represents R^{5a}, then R^{5a} preferably represents C₁₋₆ (e.g. C₁₋₄) alkyl (which group may be substituted by one or more fluoro atoms, but is more preferably unsubstituted);
when R^{5g} represents R^{5a}, then R^{5a} preferably represents unsubstituted C₁₋₄ (e.g. C₁₋₃) alkyl.

Provided that at least one of L² and L³ represents —C(O)-A¹⁷- (e.g. —C(O)—), then particularly preferred:

L² groups (i.e. when L³ represents —C(O)-A¹⁷-) include a single bond, or, L² preferably represents —O—, —N(H)—, —N(H)C(O)— and —N(H)S(O)₂— (especially preferred are —O— linker groups); and
L³ groups (i.e. when L² represents —C(O)-A¹⁷-) include —N(CH₃)—, —N(ethyl)-, —N(cyclopropylmethyl)-, —N(cyclobutylmethyl)-, —N(cyclopentylmethyl)-, —N(2-ethoxyethyl)-, —N(allyl)-, —N(2-propynyl) and —N(3-cyanopropyl)- (especially preferred are —N(CH₃)—, —N(cyclobutylmethyl)-, —N(cyclopentylmethyl)-, —N(2-ethoxyethyl)-, —N(allyl)- and —N(2-propynyl).

Preferred Y² and Y³ groups that may be mentioned include optionally substituted phenyl (e.g. halophenyl (such as monohalo- or dihalo-phenyl, in which the halo atom is/are preferably chloro and/or fluoro), trifluoromethylphenyl, tert-butylphenyl, thiomethylphenyl (i.e. methylsulfanylphenyl), methylsulfinylphenyl, methylsulfonylmethylphenyl, hydroxyphenyl, n-butoxyphenyl) and thienyl (e.g. 2-thienyl; which is preferably unsubstituted). Especially preferred are optionally substituted phenyl groups (e.g. chlorophenyl and trifluoromethylphenyl).

Particularly preferred phenyl groups that Y² and Y³ may represent include unsubstituted phenyl, 4-chlorophenyl, 3-chlorophenyl, 4-trifluoromethylphenyl, 3-trifluoromethylphenyl, 3,4-difluorophenyl, 4-tert-butylphenyl, 2-thiomethylphenyl (or 2-methylsulfanylphenyl, i.e. (2-SCH₃)phenyl), 2-methylsulfinylphenyl (i.e. (2-S(O)CH₃)phenyl), methylsulfonylmethylphenyl (i.e. (2-S(O)₂CH₃)phenyl), 2-hydroxy-5-chlorophenyl and 4-n-butoxyphenyl. Especially preferred are unsubstituted phenyl and chlorophenyl (e.g. 4-chlorophenyl and 4-trifluoromethylphenyl).

Preferred substituents on Y² and Y³ groups (e.g. when they represent aryl or heteroaryl) include halo (e.g. chloro or fluoro), C₁₋₆ (e.g. C₁₋₄) alkyl (such as methyl or butyl, e.g. n- or t-butyl; which alkyl group is optionally substituted by one or more fluoro atoms, so forming for example, a —CF₃ group), —S—C₁₋₃ alkyl (e.g. —S—CH₃), —S(O)—C₁₋₃ alkyl (e.g. —S(O)CH₃), —S(O)₂—C₁₋₃ alkyl (e.g. —S(O)₂CH₃), hydroxy (i.e. —OH), —O—C₁₋₆ (e.g. —O—C₁₋₄) alkyl (e.g. —O-n-butyl). Especially preferred substituents on such Y² and Y³ groups are halo (e.g. chloro) and C₁₋₂ alkyl (e.g. methyl) optionally (and preferably) substituted by one or more fluoro atoms (so forming, for example, a trifluoromethyl group).

Particularly preferred compounds of the invention include those of the following formula:

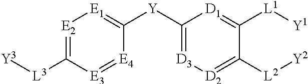

wherein
Y represents —C(O)— or —C(═N—OR²⁸)—;
R²⁸ represents hydrogen or C₁₋₃ alkyl;
either: one or two of D₁, D₂ and D₃ represents —N═; or one or two of E₁, E₂, E₃ and E₄ represent(s) —N═ (i.e. either the D₁ to D₃-containing ring or the E₁ to E₄-containing ring contains one or two —N═ moieties);
either:
(i) one of E₁, E₂, E₃ and E₄ (e.g. E₁ or E₄) represents —N═ and the others independently represent —C(R²)═; and D₁, D₂ and D₃ each independently represent —C(R¹)═;
(ii) one of D₁, D₂ and D₃ (e.g. D₁ or D₃) represents —N═ and the others independently represent —C(R¹)═; and E₁, E₂, E₃ and E₄ each independently represent —C(R²)═; or
(iii) two of E₁, E₂, E₃ and E₄ (e.g. E₁ and E₄) represent —N═ and the others independently represent —C(R²)═; and D₁, D₂ and D₃ each independently represent —C(R¹)═;
each R¹ and R² independently represent H;
Y¹ represents —C(O)OR^{9a};
R^{9a} represents: (i) hydrogen; or (ii) C₁₋₈ alkyl optionally substituted by one or more substituents selected from G¹ and/or Z¹ (but preferably unsubstituted);
L¹ represents a single bond;
one of L² and L³ (e.g. L²) represents —C(O)-A¹⁷ (e.g. —C(O)—, —C(O)—CH₂— or —C(O)-cyclopropylene-, i.e. —C(O)—C-(—CH₂—CH₂—)-) and the other is as defined herein;
L² represents —C(O)-A¹⁷ (e.g. —C(O)—, —C(O)—CH₂— or —C(O)-cyclopropylene-, i.e. —C(O)—C-(—CH₂—CH₂—)-) or, when L³ represents —C(O)-A¹⁷-, L² may represent a single bond, —OA¹⁷-, —N(R^{17a})-A¹⁶ (e.g. —N(R^{17a})—CH₂—, —N(R^{17a})—, —N(R^{17a})—C(O)— or —N(R^{17a})—S(O)₂—), —S— or —S(O)—;
L³ represents (when L² represents —C(O)-A¹⁷-) a single bond, —N(R^{17a})-A¹⁶- (e.g. —N(R^{17a})—) or —OA¹⁷ (e.g. —OCH₂—);
A¹⁶ represents —CH₂— or, preferably, a direct bond, —C(O)— or —S(O)₂—;
A¹⁷ represents a direct bond or —C(R^{y8})(R^{y9})— (in which R^{y8} and R^{y9} represent hydrogen, or, are linked together to form a cyclopropyl group);
R^{17a} represents hydrogen or C₁₋₆ alkyl (e.g. methyl, ethyl, propyl, cyclopropylmethyl, cyclobutylmethyl, cyclopentylmethyl, allyl and/or propynyl) optionally substituted (e.g. terminally substituted) by one or more (e.g. one) substituent(s) selected from fluoro, —CN, —OR¹⁹ (e.g. —OCH₂CH₃), heterocycloalkyl (which may be attached via a single common carbon atom; e.g oxetanyl), or aryl (e.g. phenyl; so forming e.g. a benzyl group);
Y² represents acyclic C₁₋₆ (e.g. C₄₋₆) alkyl or Y² more preferably represents: (i) phenyl; (ii) 5- or, preferably, 6-membered heteroaryl (e.g. in which there is preferably one heteroatom, preferably selected from nitrogen, oxygen and sulfur, so forming e.g. thienyl or, preferably, pyridyl); (iii) 9- or 10-membered bicyclic heteroaryl group (e.g. consisting of a benzene ring fused to a 5- or 6-membered heteroaryl or heterocycloalkyl group, so forming e.g. 3,4-methylenedioxyphenyl or 3,4-ethylenedioxyphenyl); (iv) C₃₋₈ (e.g. C₅₋₆) cycloalkyl; (v) or a 4- to 8-membered (e.g. 5- or 6-membered) heterocycloalkyl group (e.g. piperidinyl, tetrahydropyranyl, tetrahydrothiopyranyl or the 1,1-dioxo derivative thereof, or, tetrahydrofuranyl), all of which groups are optionally substituted by one or more substituents selected from A (or which alkyl and heterocycloalkyl groups are optionally substituted by one or more substituents selected from G¹ and Z¹);
when Y² or Y³ represent alkyl, then such groups are preferably cycloalkyl;
Y³ may represents a group as defined above for Y² (provided that at least one of Y² and Y³ represent an aromatic group), but Y³ preferably represents phenyl optionally substituted by one or more substituents selected from A;
A represents aryl or heteroaryl (e.g. phenyl or pyridyl; both of which aryl and heteroaryl groups are optionally substituted by one or more B substituents) or A more preferably represents G¹ or C₁₋₄ (e.g. C₁₋₂) alkyl (e.g. tert-butyl or methyl) optionally substituted by one or more substituents selected from G¹ (preferably A only represents an aryl (e.g. phenyl) substituent when it is on a Y² or Y³ (e.g. Y²) group that is an aromatic group, i.e. aryl or heteroaryl);
G¹ represents halo (e.g. chloro, fluoro or bromo), —CN, —NO₂, —OR^{5e}, —S(O)ₘR^{5g} or —S(O)₂N(R^{6e})R^{7e};
B represents halo (e.g. chloro or fluoro);
m represents 0, 1 or 2;
R^{5e} represents hydrogen, C₁₋₄ alkyl (which alkyl group is optionally substituted by one or more halo (e.g. fluoro) atoms; which alkyl group includes part-cyclic alkyl groups), or aryl (e.g. phenyl) or heteroaryl (e.g. pyridyl), which latter two aryl and heteroaryl groups are each optionally substituted by one or more (e.g. one) substituent(s) selected from fluoro, chloro and —CN;
R^{5g} represents C₁₋₄ alkyl (e.g. methyl);
R^{6e} and R^{7e} independently represent hydrogen or, preferably, C₁₋₂ alkyl (e.g. methyl);
Z¹ represents, on each occasion when used herein, ═O;
A substituents (i.e. on Y² or Y³ groups) include halo (e.g. chloro or fluoro), cyano, —NO₂, trifluoromethyl, methoxy, ethoxy, trifluoromethoxy, hydroxy, phenoxy (e.g. cyano-phenoxy, 2,4-difluoro-phenoxy, 2-chloro-phenoxy or 2-fluoro-phenoxy), 3-hydroxypropyl, methylsulfonyl, methylsulfanyl, methylsulfinyl and pyridyloxy (e.g. 3-pyridyloxy).

For the avoidance of doubt, all individual features (e.g. preferred features) mentioned herein may be taken in isolation or in combination with any other feature (including preferred feature) mentioned herein (hence, preferred features may be taken in conjunction with other preferred features, or independently of them).

Other preferred compounds of the invention that may be mentioned include those in which:

Y represents —C(O)—;
one of E₁, E₂, E₃ and E₄ (e.g. E₁ or E₄) represents —N═, and the others independently represent —C(R²)═; and D₁, D₂ and D₃ each independently represent —C(R¹)═;
each R¹ and R² independently represent H;
Y¹ represents —C(O)OR^{9a};
R^{9a} represents hydrogen;
L¹ represents a single bond;
L² represents —C(O)-A¹⁷- (especially, —C(O)—);
L³ represents —N(R^{17a})-A¹⁶-, —C(O)-A¹⁷- (especially, —C(O)—) or —C(R^{y4})(R^{y5})-A¹⁶-;
A¹⁶ represents a direct bond;

R^{y4} and R^{y5} independently represent hydrogen;

A¹⁷ represents a direct bond or —C(R^{y8})(R^{y9})— (in which R^{y8} and R^{y9} independently represent hydrogen, C₁₋₂ alkyl (e.g. methyl) or, are linked together to form a 3- to 6-membered cycloalkyl group, e.g. cyclopropyl, cyclopentyl or cyclohexyl);
R^{17a} represents C₁₋₆ alkyl (e.g. methyl, cyclopropylmethyl)
Y² may also represent, in addition to the groups mentioned hereinbefore: a fused and/or bridged C₆₋₁₂ cycloalkyl group, such as an adamantyl group or a bicyclo[2.2.1]heptanyl group; a C₁₋₆ acyclic alkyl group (e.g. methyl, ethyl, —CH₂-t-butyl or ethyl) or C₃₋₆ cycloakyl (e.g. cyclopropyl or cyclopentyl), which latter two groups are optionally substituted by one or more substituents selected from G¹;
G¹ may also represent, in addition to the groups mentioned hereinbefore, R^{5a} or —C(O)R^{5b};
R^{5a} may also represent, in addition to the groups mentioned hereinbefore, C₁₋₆ alkyl (e.g. C₃₋₅ cycloalkyl, such as cyclohexyl) or aryl (e.g. phenyl), preferably unsubstituted;
R^{5b} represents hydrogen.

Other preferred compounds of the invention that may be mentioned include those in which:

one of Y² and Y³ represents aryl or hetoaryl (both of which are optionally substituted by one or more (e.g. one or two) substitutent(s) selected from A) and the other represents C₁₋₁₂ alkyl (e.g. acyclic C₁₋₆ alkyl, C₃₋₆ cycloalkyl or bridged C₆₋₁₂ cycloalkyl, e.g. tert-butyl, n-hexyl, cyclohexyl, cyclopentyl, adamantyl, bicyclo[2.2.1]heptanyl, —CH₂-t-butyl, cyclopropyl, methyl or ethyl; optionally substituted by one or more (e.g. one) substituent(s) selected from G¹), a 5- to 6-membered heterocycloalkyl group (e.g. tetrahydrofuranyl), aryl or hetoaryl, both of which latter two groups are optionally substituted by one or more (e.g. one or two) substitutent(s) selected from A; or
Y² and Y³ independently represent aryl or heteroaryl, both of which are optionally substituted by one or more (e.g. one or two) substitutent(s) selected from A;
when Y² or Y³ represents aryl, it is preferably (optionally substituted) phenyl;
when Y² or Y³ represents heteroaryl, it is preferably a 5 to 6-membered monocyclic group or a 9- to 10-membered bicyclic group (e.g. benzene fused to a 5-6 membered heteroaryl group), for instance (optionally substituted) thienyl (e.g. 2-thienyl), 3,4-methylenedioxyphenyl or 3,4-ethylenedioxyphenyl;
A represents G¹ or C₁₋₃ alkyl (e.g. methyl or ethyl) optionally substituted by one or more G¹ groups (e.g. in which G¹ is fluoro, so forming e.g. a —CF₃ group, or chloro, so forming e.g. dichloromethyl);
G¹ (e.g. when A represents G¹) represents halo (e.g. chloro or fluoro), R^{5a}, —C(O)R^{5b}, —NO₂ or —OR^{5e};
G¹ may also represent (e.g. when Y² or Y³ represent alkyl substituted by G¹) R^{5a}, in which R^{5a} represents C₃₋₆ cycloalkyl (e.g. cyclohexyl or cyclopentyl) or aryl (e.g. phenyl; preferably unsubstituted);

R^{5e} represents hydrogen or R^{5a};

R^{5b} represents R^{5a} or, preferably, hydrogen;
R^{5a} represents C₁₋₆ (e.g. C₁₋₃) alkyl (e.g. methyl or ethyl) optionally substituted by one or more fluoro atoms;
Y² represents unsubstituted phenyl, methoxyphenyl, ethoxyphenyl, trifluoromethylphenyl, nitrophenyl, halophenyl, diethoxyphenyl, thienyl (e.g. 2-thienyl), 3,4-methylenedioxyphenyl, 3,4-ethylenedioxyphenyl, trifluoromethoxyhenyl, dihalomethylphenyl or hydroxyphenyl;
Y³ represents halophenyl, dihalophenyl, trifluoromethylphenyl, alkylphenyl (e.g. methylphenyl or ethylphenyl) or halo-alkyl-phenyl.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which Y represents —C(O)—, oxidation of a compound of formula II,

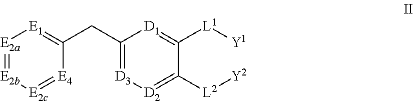

wherein ring E₁, E₂ₐ, E_{2b}, E_{2c}, E_{2d}, E₄, D₁, D₂, D₃, L¹, Y¹, L² and Y² are as hereinbefore defined, in the presence of a suitable oxidising agent;
(ia) for compounds of formula I in which Y represents —C(O)—, oxidation of a compound of formula IIA,

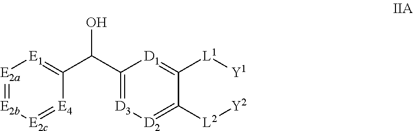

wherein E₁, E₂ₐ, E_{2b}, E_{2c}, E_{2d}, E₄, D₁, D₂, D₃, L¹, Y¹, L² and Y² are as hereinbefore defined, in the presence of a suitable oxidising agent, for example, pyridinium chlorochromate (PCC) or the like (e.g. pyridinium dichromate; PDC);
(ii) for compounds of formula I in which L² or L³ represents —N(R^{17a})A¹⁶- (and the other represents —C(O)-A¹⁷-) in which R^{17a} represents H (and, preferably, Y is —C(O)— or R²⁸ is C₁₋₆ alkyl optionally substituted by one or more halo atoms), reaction of a compound of formula III,

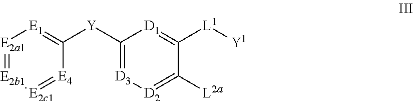

or a protected derivative thereof (e.g. an amino-protected derivative or a keto-protecting group, such as a ketal or thioketal) wherein one of E₂ₐ₁, E_{2b1}, E_{2c1} represents —C(-L^{3a})= and the other two respectively represent E₂ and E₃, one of L^{2a} and L^{3a} represents —NH₂ and the other represents —C(O)-A¹⁷-Y² or —C(O)-A¹⁷-Y³ as appropriate, and Y, E₁, E₂, E₃, E₄, D₁, D₂, D₃, L¹ and Y¹ are as hereinbefore defined, with:
(A) when A¹⁶ represents —C(O)N(R^{17b})—, in which R^{17b} represents H:
(a) a compound of formula IV,

Y^{a}—N═C═O IV
; or
(b) with CO (or a reagent that is a suitable source of CO (e.g. Mo(CO)₆ or Co₂(CO)₈)) or a reagent such as phosgene or triphosgene in the presence of a compound of formula V,

Y^{a}—NH₂ V

wherein, in both cases, Y^{a} represents Y² or Y³ (as appropriate/required) as hereinbefore defined. For example, in the case of (a) above, in the presence of a suitable solvent (e.g. THF, dioxane or diethyl ether) under reaction conditions known to those skilled in the art (e.g. at room temperature). In the case of (b), suitable conditions will be known to the skilled person, for example the reactions may be carried out in the presence of an appropriate catalyst system (e.g. a palladium catalyst), preferably under pressure and/or under microwave irradiation conditions. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof). The skilled person will appreciate that for preparation of compounds of formula I in which -L-Y² represents —N(H)C(O)N(H)—Y² and -L-Y³ represents —N(H)C(O)N(H)—Y³ and Y² and Y³ are different, two different compounds of formula IV or V (as appropriate) will need to be employed in successive reaction steps. For the preparation of such compounds starting from compounds of formula III in which both of L^{2a} and L^{3a} represent —NH₂, then mono-protection (at a single amino group) followed by deprotection may be necessary, or the reaction may be performed with less than 2 equivalents of the compound of formula IV or V (as appropriate);
(B) when A¹⁶ represents a direct bond, with a compound of formula VI,

Y^{a}-L^{a} VI

wherein L^{a} represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ (or a protected derivative thereof, e.g. an alkyl protected derivative, so forming, for example a 4,4,5,5-tetramethyl-1,3,2-dioxaborolan-2-yl group) and Y^{a} is as hereinbefore defined, for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when Y^{a} represents phenyl and L^{a} represents bromo, i.e. bromobenzene). This reaction may be carried out at room temperature or above (e.g. at a high temperature, such as the reflux temperature of the solvent system that is employed) or using microwave irradiation;
(C) when A¹⁶ represents —S(O)₂—, —C(O)— or —C(O)—C(R^{y6})(R^{y7})—, with a compound of formula VII,

Y^{a}-A^{16a}-L^{a} VII

wherein A^{16a} represents —S(O)₂—, —C(O)— or —C(O)—C(R^{y6})(R^{y7})—, and Y^{a} and L^{a} are as hereinbefore defined, and L^{a} is preferably, bromo or chloro, under reaction conditions known to those skilled in the art, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)-amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine);
(iii) for compounds of formula I in which one of L² and L³ represents —C(O)-A¹⁷- and the other represents —N(R^{17a})C(O)N(R^{17b})—, in which R^{17a} and R^{17b} represent H, and, preferably, Y is —C(O)— or R²⁸ is C₁₋₆ alkyl optionally substituted by one or more halo atoms, reaction of a compound of formula VIII,

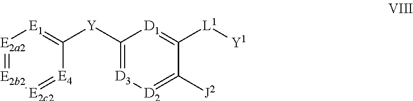

wherein one of E₂ₐ₂, E_{2b2}, E_{2c2} represents —C(-J¹)= and the other two respectively represent E₂ and E₃, one of J¹ and J² represents —N═C═O and the other represents —C(O)-A¹⁷-Y² or —C(O)-A¹⁷-Y³ (as appropriate), and Y, E₁, E₂, E₃, E₄, D₁, D₂, D₃, L¹ and Y¹ are as hereinbefore defined, with a compound of formula V as hereinbefore defined, under reaction conditions known to those skilled in the art, such as those described hereinbefore in respect of process step (ii)(A)(b) above;
(iv) for compounds of formula I in which, preferably, Y is —C(O)— or R²⁸ is C₁₋₆ alkyl optionally substituted by one or more halo atoms, reaction of a compound of formula IX,

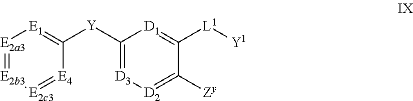

wherein one of E₂ₐ₃, E_{2b3}, E_{2c3} represents —C(—Z^{x})═ and the other two respectively represent E₂ and E₃, at least one of Z^{x} and Z^{y} represents a suitable leaving group and the other may also independently represent a suitable leaving group, or, Z^{y} may represent -L²-Y² and Z^{x} may represent -L³-Y³, in which the suitable leaving group may independently be fluoro or, preferably, chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃ or diazonium salts, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), and Y, E₁, E₂, E₃, E₄, D₁, D₂, D₃, L¹, Y¹, L², Y², L³ and Y³ are as hereinbefore defined, with a (or two separate) compound(s) (as appropriate/required) of formula X,

Y^{a}-L^{x}-H X

wherein L^{x} represents L² or L³ (as appropriate/required; in which they are preferably and independently selected from —N(R^{17a})-A¹⁶- and —OA¹⁷-, provided that at least one of L² and L³ represent —C(O)A¹⁷-Y^{a}), and Y^{a} is as hereinbefore defined, under suitable reaction conditions known to those skilled in the art, e.g. such as those hereinbefore described in respect of process (ii) above (e.g. (II)(B)), for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). Alternatively, for example, when L² or L³ represent —O— or —S-(and hence the compound of formula X is an alcohol, e.g. a phenol or a thiol, e.g. thiophenol), or, L² or L³ represent single bonds, and Y² or Y³ are to be attached to the requisite biaryl moiety (of the compounds of the invention, which may alternatively be termed the diaryl; for the purposes herein both terms may be interchangeably employed) via a heteroatom, e.g. nitrogen), the reaction may be performed in the presence of a mixture of KF/Al₂O₃ (e.g. in the presence of a suitable solvent such as acetonitrile, at elevated temperature, e.g. at about 100° C.; in this instance the leaving group that Z^{x} or Z^{y} may represent in the compound of formula IX is preferably fluoro). The skilled person will appreciate that when compounds of formula I in which L² and L³ are different are required, then reaction with different compounds of formula X may be required;
(v) compounds of formula I in which there is a R^{17a} or R^{17b} group present that does not represent hydrogen (or if there is R⁵, R⁶, R⁷, R⁸, R⁹, R¹¹, R¹², R¹³, R¹⁴, R¹⁶, R¹⁷ or R¹⁸ group present, which is attached to a heteroatom such as nitrogen or oxygen, and which does/do not represent hydrogen), may be prepared by reaction of a corresponding compound of formula I in which such a group is present that does represent hydrogen with a compound of formula XI,

R^{wy}-L^{b} XI

wherein R^{wy} represents either R^{17a} or R^{17b} (as appropriate) as hereinbefore defined provided that it does not represent hydrogen (or R^{wy} represents a R⁵ to R¹⁸ group in which those groups do not represent hydrogen), and L^{b} represents a suitable leaving group such as one hereinbefore defined in respect of L^{a} or —Sn(alkyl)₃ (e.g. —SnMe₃ or —SnBu₃), or a similar group known to the skilled person, under reaction conditions known to those skilled in the art, for example such as those described in respect of process step (ii)(C) above. The skilled person will appreciate that various groups (e.g. primary amino groups) may need to be mono-protected and then subsequently deprotected following reaction with the compound of formula XI;
(vi) for compounds of formula I that contain only saturated alkyl groups, reduction of a corresponding compound of formula I that contains an unsaturation, such as a double or triple bond, in the presence of suitable reducing conditions, for example by catalytic (e.g. employing Pd) hydrogenation;
(vii) for compounds of formula I in which Y¹ represents —C(O)OR^{9a}, in which R^{9a} represent hydrogen (or other carboxylic acid or ester protected derivatives (e.g. amide derivatives)), hydrolysis of a corresponding compound of formula I in which R^{9a} does not represent H, under standard conditions, for example in the presence of an aqueous solution of base (e.g. aqueous 2M NaOH) optionally in the presence of an (additional) organic solvent (such as dioxane or diethyl ether), which reaction mixture may be stirred at room or, preferably, elevated temperature (e.g. about 120° C.) for a period of time until hydrolysis is complete (e.g. 5 hours). Alternatively, non-hydrolytic means may be employed to convert esters to acids e.g. by hydrogentation or oxidation (e.g. for certain benzylic groups) known to those skilled in the art;
(viii) for compounds of formula I in which Y¹ represent —C(O)OR^{9a} and R^{9a} does not represent H:
(A) esterification (or the like) of a corresponding compound of formula I in which R^{9a} represents H; or
(B) trans-esterification (or the like) of a corresponding compound of formula I in which R^{9a} does not represent H (and does not represent the same value of the corresponding R^{9a} group in the compound of formula I to be prepared),
   under standard conditions in the presence of the appropriate alcohol of formula XII,

R^{9za}OH XII

in which R^{9za} represents R^{9a} provided that it does not represent H, for example further in the presence of acid (e.g. concentrated H₂SO₄) at elevated temperature, such as at the reflux temperature of the alcohol of formula XII;
(ix) for compounds of formula I in which Y¹ preferably represents —C(O)OR^{9a}, in which R^{9a} is other than H, and L¹ is as hereinbefore defined, provided that it does not represent —(CH₂)ₚ-Q-(CH₂)_{q}— in which p represents 0 and Q represents —O—, and, preferably, Y is —C(O)— or R²⁸ is C₁₋₆ alkyl optionally substituted by one or more halo atoms, reaction of a compound of formula XIII,

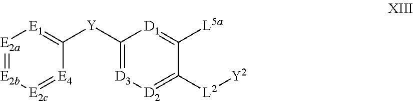

wherein L^{5a} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and Y, E₁, E₂ₐ, E_{2b}, E_{2c}, E₄, D₁, D₂, D₃, L² and Y² are as hereinbefore defined (the skilled person will appreciate that the compound of formula XIII in which L^{5a} represents an alkali metal (e.g. lithium), a Mg-halide or a zinc-based group may be prepared from a corresponding compound of formula XIII in which L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XIV,

L⁶-L^{xy}-Y^{b} XIV

wherein L^{xy} represents L¹ (provided that it does not represent —(CH₂)ₚ-Q-(CH₂)_{q}— in which p represents 0 and Q represents —O—) and Y^{b} represents —C(O)OR^{9a}, in which R^{9a} is other than H, and L⁶ represents a suitable leaving group known to those skilled in the art, such as C₁₋₃ alkoxy and, preferably, halo (especially chloro or bromo). For example, the compound of formula XIV may be Cl—C(O)OR^{9a}. The reaction may be performed under standard reaction conditions, for example in the presence of a polar aprotic solvent (e.g. THF or diethyl ether);
(x) compounds of formula I in which L¹ preferably represents a single bond, and Y¹ represents 5-tetrazolyl (and, preferably, Y is —C(O)— or R²⁸ is C₁₋₆ alkyl optionally substituted by one or more halo atoms), may be prepared in accordance with the procedures described in international patent application WO 2006/077366;
(xi) for compounds of formula I in which L¹ represents a single bond, and Y¹ represents —C(O)OR^{9a} in which R^{9a} is H, (and, preferably, Y is —C(O)— or R²⁸ is C₁₋₆ alkyl optionally substituted by one or more halo atoms), reaction of a compound of formula XIII as hereinbefore defined but in which L^{5a} represents either:
(I) an alkali metal (for example, such as one defined in respect of process step (ix) above); or
(II) —Mg-halide,
   with carbon dioxide, followed by acidification under standard conditions known to those skilled in the art, for example, in the presence of aqueous hydrochloric acid;
   (xii) for compounds of formula I in which L¹ represents a single bond, and Y¹ represents —C(O)OR^{9a} (and, preferably, Y is —C(O)— or R²⁸ is C₁₋₆ alkyl optionally substituted by one or more halo atoms), reaction of a corresponding compound of formula XIII as hereinbefore defined but in which L^{5a} is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or Co₂(CO)₈)), in the presence of a compound of formula XV,

R^{9a}OH XV

wherein R^{9a} is as hereinbefore defined, and an appropriate catalyst system (e.g. a palladium catalyst, such as PdCl₂, Pd(OAc)₂, Pd(Ph₃P)₂Cl₂, Pd(Ph₃P)₄, Pd₂(dba)₃ or the like) under conditions known to those skilled in the art;
(xiii) for compounds of formula I in which Y represents —C(O)—, reaction of either a compound of formula XVI or XVII,

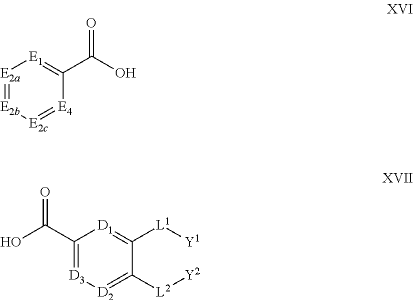

respectively with a compound of formula XVIII or XIX,

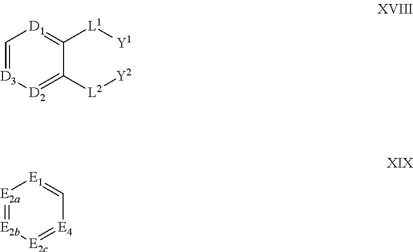

wherein (in all cases) E₁, E₂ₐ, E_{2b}, E_{2c}, E₄, D₁, D₂, D₃, L¹, Y¹, L² and Y² are as hereinbefore defined, in the presence of a suitable reagent that converts the carboxylic acid group of the compound of formula XVI or XVII to a more reactive derivative (e.g. an acid chloride or acid anhydride, or the like; which reactive derivative may itself be separately prepared and/or isolated, or where such a reactive derivative may be prepared in situ) such as POCl₃, in the presence of ZnCl₂, for example as described in Organic and Biomolecular Chemistry (2007), 5(3), 494-500 or, more preferably, PCl₃, PCl₅, SOCl₂ or (COCl)₂. Alternatively, such a reaction may be performed in the presence of a suitable catalyst (for example a Lewis acid catalyst such as SnCl₄), for example as described in Journal of Molecular Catalysis A: Chemical (2006), 256(1-2), 242-246 or under alternative Friedel-crafts acylation reaction conditions (or variations thereupon) such as those described in Tetrahedron Letters (2006), 47(34), 6063-6066; Synthesis (2006), (21), 3547-3574; Tetrahedron Letters (2006), 62(50), 11675-11678; Synthesis (2006), (15), 2618-2623; Pharmazie (2006), 61(6), 505-510; and Synthetic Communications (2006), 36(10), 1405-1411. Alternatively, such a reaction between the two relevant compounds may be performed under coupling reaction conditions (e.g. Stille coupling conditions), for example as described in Bioorganic and Medicinal Chemistry Letters (2004), 14(4), 1023-1026;
(xiv) for compounds of formula I in which Y represents —C(O)—, reaction of either a compound of formula XX or XXI,

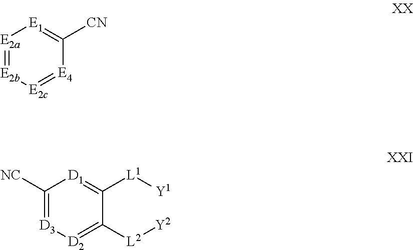

with a compound of formula XXII or XXIII,

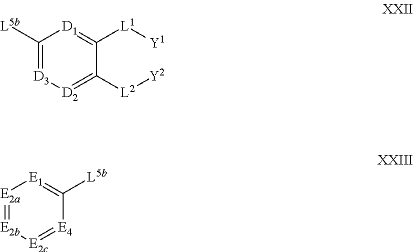

respectively, wherein L^{5b} represents L^{5a} as hereinbefore defined, and which may therefore represent —B(OH)₂ (or a protected derivative thereof), an alkali metal (such as lithium) or a —Mg-halide (such as —MgI or, preferably, —MgBr), and (in all cases) E₁, E₂ₐ, E_{2b}, E_{2c}, E₄, D₁, D₂, D₃, L¹, Y¹, L² and Y² are as hereinbefore defined, and (in the case of compounds of formulae XXII and XXIII), for example in the presence of a suitable solvent, optionally in the presence of a catalyst, for example, as described in Organic Letters (2006), 8(26), 5987-5990. Compounds of formula I may also be obtained by performing variations of such a reaction, for example by performing a reaction of a compound of formula XX or XXI respectively with a compound of formula XVIII or XIX as hereinbefore defined, for example under conditions described in Journal of Organic Chemistry (2006), 71(9), 3551-3558 or US patent application US 2005/256102;
(xv) for compounds of formula I in which Y represents —C(O)—, reaction of an activated derivative of a compound of formula XVI or XVII as hereinbefore defined (for example an acid chloride; the preparation of which is hereinbefore described in process step (xiii) above), with a compound of formula XXII or XXIII (as hereinbefore defined), respectively, for example under reaction conditions such as those hereinbefore described in respect of process step (xiii) above;
(xvi) for compounds of formula I in which Y represents —C(═N—OR²⁸)—, reaction of a corresponding compound of formula I in which Y represents —C(O)—, with a compound of formula XXIIIA,

H₂N—O—R²⁸ XXIIIA

wherein R²⁸ is represents hydrogen or C₁₋₆ alkyl optionally substituted by one or more halo atoms, under standard condensation reaction conditions, for example in the presence of an anhydrous solvent (e.g. dry pyridine, ethanol and/or another suitable solvent);
(xvii) for compounds of formula I in which Y represents —C(═N—OR²⁸)— and R²⁸ represents C₁₋₆ alkyl optionally substituted by one or more halo atoms, reaction of a corresponding compound of formula I, in which R²⁸ represents hydrogen, with a compound of formula XXIIIB,

R^{28a}-L⁷ XXIIIB

wherein R^{28a} represents R²⁸, provided that it does not represent hydrogen and L⁷ represents a suitable leaving group, such as one hereinbefore defined in respect of L^{a} (e.g. bromo or iodo), under standard alkylation reaction conditions, such as those hereinbefore described in respect of process step (ii) (e.g. (ii)(C)).

Compounds of formula II may be prepared by reaction of a compound of formula XVIII with a compound of formula XIX, both as hereinbefore defined, with formaldehyde (e.g. in the form of paraformaldehyde or an aqueous solution of formaldehyde such as a 3% aqueous solution), for example under acidic conditions (e.g. in the presence of aqueous HCl) at or above room temperature (e.g. at between 50° C. and 70° C.). Preferably, the formaldehyde is added (e.g. slowly) to an acidic solution of the compound of formula XVIII at about 50° C., with the reaction temperature rising to about 70° C. after addition is complete. When acidic conditions are employed, precipitation of the compound of formula II may be effected by the neutralisation (for example by the addition of a base such as ammonia). Compounds of formula I may also be prepared in accordance with such a procedure, for example under similar reaction conditions, employing similar reagents and reactants.

Compounds of formula IIA may be prepared by reaction of a compound of formula XXIIIC or XXIIID,

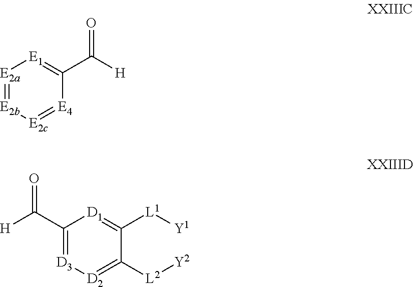

wherein E₁, E₂ₐ, E_{2b}, E_{2c}, E₄, D₁, D₂, D₃, L¹, L², Y¹ and Y² are as hereinbefore defined, with a compound of formula XXII or XXIII, respectively, for example under reaction conditions such as those hereinbefore described in respect of preparation of compounds of formula I (process step (xiii)).

Compounds of formulae III, VIII, IX and XIII in which Y represents —C(O)—, may be prepared by oxidation of a compound of formulae XXIV, XXV, XXVI and XXVII, respectively,

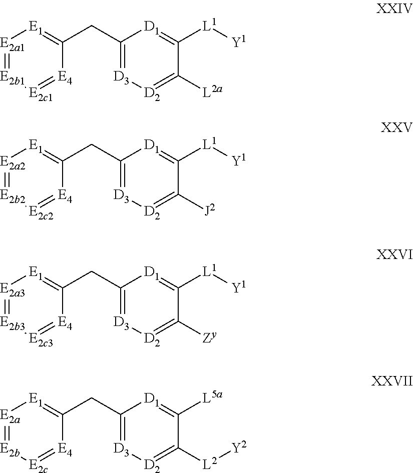

wherein E₁, E₂ₐ₁, E_{2b1}, E_{2c1}, E₂ₐ₂, E_{2b2}, E_{2c2}, E₂ₐ, E_{2b}, E_{2c}, E₄, D₁, D₂, D₃, L¹, Y¹, L^{2a}, J², Z^{y}, L², Y² and L^{5a} are as hereinbefore defined, under standard oxidation conditions known to those skilled in the art, for example such as those hereinbefore described in respect of preparation of compounds of formula I (process step (i) above). The skilled person will appreciate that, similarly, compounds of formulae XXIV, XXV, XXVI and XXVII may be prepared by reduction of corresponding compounds of formulae III, VIII, IX and XIII, under standard reaction conditions, such as those described herein.

Compounds of formula III in which Y represents —C(O)—, or, preferably, compounds of formula XXIV (or protected, e.g. mono-protected derivatives thereof) may be prepared by reduction of a compound of formula XXVIII,

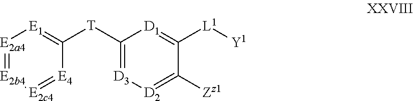

wherein T represents —C(O)— (in the case where compounds of formula III are to be prepared) or, preferably, —CH₂— (in the case where compounds of formula XXIV are to be prepared), one of E₂ₐ₄, E_{2b4} and E_{2c4} represents —C(—Z^{z2})═, and the others respectively represent E₂ and E₃, one of Z^{z1} and Z^{z2} represents —N₃ or —NO₂, and the other represents —C(O)-A¹⁷-Y² or —C(O)-A¹⁷-Y² (as appropriate), under standard reaction conditions known to those skilled in the art, in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane). The skilled person will appreciate that where the reduction is performed in the presence of a —C(O)— group (e.g. when T represents —C(O)—), a chemoselective reducing agent may need to be employed.

Compounds of formula III in which one of L^{2a} and L^{3a} represents —NH₂ (or a protected derivative thereof) may also be prepared by reaction of a compound of formula IX as defined above, with ammonia, or preferably with a protected derivative thereof (e.g. benzylamine or Ph₂C═NH), under conditions such as those described hereinbefore in respect of preparation of compounds of formula I (process step (iv) above).

Compounds of formulae III, IX, XXIV or XXV in which L¹ represents a single bond, and Y¹ represents —C(O)OR^{9a}, may be prepared by:

(I) reaction of a compound of formula XXIX,

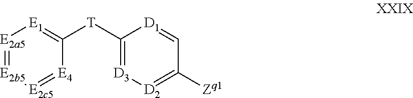

wherein one of E₂ₐ₅, E_{2b5} and E_{2c5} represents —C(—Z^{q2})═, and the others respectively represent E₂ and E₃, Z^{q1} and Z^{q2} respectively represent Z^{y} and Z^{x} (in the case of preparation of compounds of formulae IX or XXV), they respectively represent L^{2a} and L^{3a} (in the case of preparation of compounds of formulae III or XXIV), and E₁, E₂, E₃, E₄, D₁, D₂, D₃, Z^{x}, Z^{y}, L^{2a}, L^{3a} and T are as hereinbefore defined, with a suitable reagent such as phosgene or triphosgene in the presence of a Lewis acid, followed by reaction in the presence of a compound of formula XV as hereinbefore defined, hence undergoing a hydrolysis or alcoholysis reaction step;
(II) for such compounds in which R^{9a} represents hydrogen, formylation of a compound of formula XXIX as hereinbefore defined, for example in the presence of suitable reagents such as P(O)Cl₃ and DMF, followed by oxidation under standard conditions;
(III) reaction of a compound of formula XXX,

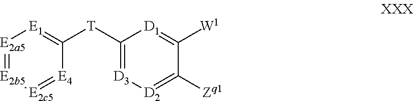

wherein W¹ represents a suitable leaving group such as one defined by Z^{x} and Z^{y} above, and E₁, E₂ₐ₅, E_{2b5}, E_{2c5}, E₄, D₁, D₂, D₃, Z^{q1} and T are as hereinbefore defined, are as hereinbefore defined, with CO (or a reagent that is a suitable source of CO (e.g. Mo(CO)₆ or Co₂(CO)₈) followed by reaction in the presence of a compound of formula XV as hereinbefore defined, under reaction conditions known to those skilled in the art, for example such as those hereinbefore described in respect of preparation of compounds of formula I (process step (ii), e.g. (ii)(A)(b) above), e.g. the carbonylation step being performed in the presence of an appropriate precious metal (e.g. palladium) catalyst;
(IV) reaction of a compound of formula XXXI,

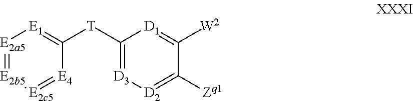

wherein W² represents a suitable group such as an appropriate alkali metal group (e.g. sodium, potassium or, especially, lithium), a —Mg-halide or a zinc-based group, and E₁, E₂ₐ₅, E_{2b5}, E_{2c5}, E₄, D₁, D₂, D₃, Z^{q1} and T are as hereinbefore defined, with e.g. CO₂ (in the case where R^{9a} in the compounds to be prepared represents hydrogen) or a compound of formula XIV in which L^{xy} represents a single bond, Y^{b} represents —C(O)OR^{9a}, in which R^{9a} is other than hydrogen, and L⁶ represents a suitable leaving group, such as chloro or bromo or a C₁₋₁₄ (such as C₁₋₆ (e.g. C₁₋₃) alkoxy group), under reaction conditions known to those skilled in the art. The skilled person will appreciate that this reaction step may be performed directly after (i.e. in the same reaction pot) the preparation of compounds of formula XXXI (which is described hereinafter).

Compounds of formula IX in which Z^{x} and/or Z^{y} represent a sulfonate group may be prepared from corresponding compounds in which the Z^{x} and Z^{y} groups represent a hydroxy group, with an appropriate reagent for the conversion of the hydroxy group to the sulfonate group (e.g. tosyl chloride, mesyl chloride, triflic anhydride and the like) under conditions known to those skilled in the art, for example in the presence of a suitable base and solvent (such as those described above in respect of process step (i), e.g. an aqueous solution of K₃PO₄ in toluene) preferably at or below room temperature (e.g. at about 10° C.).

Compounds of formulae XX and XXI may be prepared, for example, by reaction of a corresponding compound of formula XXIII or XXII, respectively (all of which are as hereinbefore defined, e.g. in which L^{5b} represents bromo or, preferably, iodo), for example, in the presence of a nucleophile that is a source of cyano ions, e.g. potassium or, preferably, copper cyanide.

Compounds of formulae XXII and XXIII in which L^{5b} represents a —Mg-halide may be prepared by reaction of a compound corresponding to a compound of formula XXII or XXIII but in which L^{5b} represents a halo group (e.g. bromo or iodo), under standard Grignard formation conditions, for example in the presence of i-PrMgCl (or the like) in the presence of a polar aprotic solvent (such as THF) under inert reaction condition, and preferably at low temperature (such as at below 0° C., e.g. at about 30° C.). The skilled person will appreciate that these compounds may be prepared in situ (see e.g. the process for the preparation of compounds of formula I (process steps (xvi) and (xvii)).

Compounds of formulae XXIIIC or XXIIID may be prepared by reaction of a corresponding compound of formula XXIII or XXII, as hereinbefore defined (and preferably one in which L^{5b} is a —Mg-halide, such as —Mg—I), with dimethylformamide (or a similar reagent for the introduction of the aldehyde group), under standard Grignard reaction conditions known to those skilled in the art (for example those described herein).

Compounds of formulae XXIX or XXX in which T represents —CH₂— may be prepared by reduction of a corresponding compound of formulae XXIX or XXX in which T represents —C(O)— (or from compounds corresponding to compounds of formulae XXIX or XXX but in which T represents —CH(OH)—), for example under standard reaction conditions known to those skilled in the art, for example reduction in the presence of a suitable reducing reagent such as LiAlH₄, NaBH₄ or trialkylsilane (e.g. triethylsilane) or reduction by hydrogenation (e.g. in the presence of Pd/C).

Alternatively, compounds of formulae XXIX or XXX in which T represents —CH₂— may be prepared by reaction of a compound of formula XXXII,

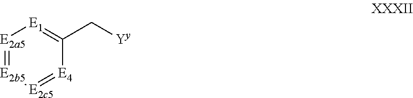

wherein Y^{y} represents a suitable group such as —OH, bromo, chloro or iodo, and E₁, E₂ₐ₅, E_{2b5}, E_{2c5} and E₄ are as hereinbefore defined, with a compound of formula XXXIII,

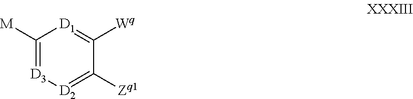

wherein M represents hydrogen and W^{q} represents hydrogen (for compounds of formula XXIX) or W¹ (for compounds of formula XXX) and D₁, D₂, D₃ and Z^{q1} are as hereinbefore defined, under standard conditions, for example in the presence of a Lewis or Brønsted acid. Alternatively, such compounds may be prepared from reaction of a compound of formula XXXII in which Y^{y} represents bromo or chloro with a compound corresponding to a compound of formula XXXIII but in which M represents —BF₃K (or the like), for example in accordance with the procedures described in Molander et al, J. Org. Chem. 71, 9198 (2006).

Compounds of formulae XXIX or XXX in which T represents —C(O)— may be prepared by reaction of a compound of formula XXXIV,

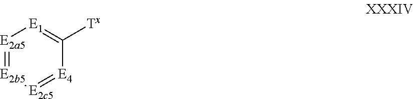

wherein T^{x} represents —C(O)Cl or —C═N—NH(t-butyl) (or the like) E₁, E₂ₐ₅, E_{2b5}, E_{2c5} and E₄ are as hereinbefore defined, with a compound of formula XXXIII, as defined above, but in which M represents hydrogen or an appropriate alkali metal group (e.g. sodium, potassium or, especially, lithium), a —Mg-halide or a zinc-based group, or, a bromo group, and D₁, D₂, D₃, Z^{q1} and W^{q} are as hereinbefore defined, under reaction conditions known to those skilled in the art. For example in the case of reaction of a compound of formula XXXIV in which T^{x} represents —C(O)Cl with a compound of formula XXXIII in which M represents hydrogen, in the presence of an appropriate Lewis acid. In the case where M represents an appropriate alkali metal group, a —Mg-halide or a zinc-based group, under reaction conditions such as those hereinbefore described in respect of preparation of compounds of formulae III, IX, XXIV or XXV (process step (IV) above) and preparation of compounds of formula XXXI (see below). In the case of a reaction of a compound of formula XXXIV in which T^{x} represents —C═N—NH(t-butyl) (or the like) with a compound of formula XXXIII in which M represents bromo, under reaction conditions such as those described in Takemiya et al, J. Am. Chem. Soc. 128, 14800 (2006).

For compounds corresponding to compounds of formula XXIX or XXX but in which T represents —CH(OH)—, reaction of a compound corresponding to a compound of formula XXXIV, but in which T^{x} represents —C(O)H, with a compound of formula XXXIII as defined above, under reaction conditions such as those hereinbefore described in respect of preparation of compounds of formulae XXIX or XXX in which T represents —C(O)—.

Compounds of formula XXXI may be prepared in several ways. For example, compounds of formula XXXI in which W² represents an alkali metal such as lithium, may be prepared from a corresponding compound of formula XXIX (in particular those in which Z^{q1} and/or Z^{q2} represents a chloro or sulfonate group or, especially, a protected —NH₂ group, wherein the protecting group is preferably a lithiation-directing group, e.g. an amido group, such as a pivaloylamido group, or a sulfonamido group, such as an arylsulfonamido group, e.g. phenylsulfonamide), by reaction with an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium coordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)), for example in the presence of a suitable solvent, such as a polar aprotic solvent (e.g. tetrahydrofuran or diethyl ether), at sub-ambient temperatures (e.g. 0° C. to −78° C.) under an inert atmosphere. Alternatively, such compounds of formula XXXI may be prepared by reaction of a compound of formula XXX in which W¹ represents chloro, bromo or iodo by a halogen-lithium reaction in the presence of an organolithium base such as t- or n-butyllithium under reaction conditions such as those described above. Compounds of formula XXXI in which W² represents —Mg-halide may be prepared from a corresponding compound of formula XXX in which W¹ represents halo (e.g. bromo), for example optionally in the presence of a catalyst (e.g. FeCl₃) under standard Grignard conditions known to those skilled in the art. The skilled person will also appreciate that the magnesium of the Grignard reagent or the lithium of the lithiated species may be exchanged to a different metal (i.e. a transmetallation reaction may be performed), for example to form compounds of formula XXXI in which W² represents a zinc-based group (e.g. using ZnCl₂).

Compounds mentioned herein (e.g. those of formulae IV, V, VA, VI, VII, X, XI, XII, XIII, XIV, XV, XVI, XVII, XVIII, XIX, XX, XXI, XXII, XXIII, XXIIIA, XXIIIB, XXV, XXVII, XXVIII, XXXII, XXXIII and XXXIV) are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further, the compounds described herein may also be prepared in accordance with synthetic routes and techniques described in international patent application WO 2006/077366.

The substituents E₁, E₂ₐ, E_{2b}, E_{2c}, E₄, D₁, D₂, D₃, L¹, Y¹, L², Y², L³ and Y³ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications (e.g. from a carboxylic acid, e.g. in the presence of H₂SO₄ and appropriate alcohol or in the presence of K₂CO₃ and alkyl iodide), etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases where Y¹ represents —C(O)OR^{9a} in which R^{9a} does not initially represent hydrogen (so providing at least one ester functional group), the skilled person will appreciate that at any stage during the synthesis (e.g. the final step), the relevant R^{9a}-containing group may be hydrolysed to form a carboxylic acid functional group (i.e. a group in which R^{9a} represents hydrogen). In this respect, the skilled person may also refer to “Comprehensive Organic Functional Group Transformations” by A. R. Katritzky, O. Meth-Cohn and C. W. Rees, Pergamon Press, 1995. Other specific transformation steps include: the reduction of a nitro group to an amino group; the hydrolysis of a nitrile group to a carboxylic acid group; standard nucleophilic aromatic substitution reactions, for example in which an iodo-, preferably, fluoro- or bromo-phenyl group is converted into a cyanophenyl group by employing a source of cyanide ions (e.g. by reaction with a compound which is a source of cyano anions, e.g. sodium, copper (I), zinc or, preferably, potassium cyanide) as a reagent (alternatively, in this case, palladium catalysed cyanation reaction conditions may also be employed); the reduction of an azido group to an amino group (e.g. in the presence of FeCl₃ trihydrate and zinc powder); and the oxidation of a sulfide to a sulfoxide or to a sulfone (e.g. conversion of a —SCH₃ substituent to a —S(O)CH₃ or —S(O)₂CH₃ substituent in the presence of a suitable oxidising agent such as Oxone or meta-chloroperbenzoic acid (MCPBA)), or the reverse reduction in the presence of a suitable reducing agent.

Other transformations that may be mentioned include: the conversion of a halo group (preferably iodo or bromo) to a 1-alkynyl group (e.g. by reaction with a 1-alkyne), which latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine); the introduction of amino groups and hydroxy groups in accordance with standard conditions using reagents known to those skilled in the art; the conversion of an amino group to a halo, azido or a cyano group, for example via diazotisation (e.g. generated in situ by reaction with NaNO₂ and a strong acid, such as HCl or H₂SO₄, at low temperature such as at 0° C. or below, e.g. at about −5° C.) followed by reaction with the appropriate reagent/nucleophile e.g. a source of the relevant reagent/anion, for example by reaction in the presence of a reagent that is a source of halogen (e.g. CuCl, CuBr or NaI), or a reagent that is a source of azido or cyanide anions, such as NaN₃, CuCN or NaCN; the conversion of —C(O)OH to a —NH₂ group, under Schmidt reaction conditions, or variants thereof, for example in the presence of HN₃ (which may be formed in by contacting NaN₃ with a strong acid such as H₂SO₄), or, for variants, by reaction with diphenyl phosphoryl azide ((PhO)₂P(O)N₃) in the presence of an alcohol, such as tert-butanol, which may result in the formation of a carbamate intermediate; the conversion of —C(O)NH₂ to —NH₂, for example under Hofmann rearrangement reaction conditions, for example in the presence of NaOBr (which may be formed by contacting NaOH and Br₂) which may result in the formation of a carbamate intermediate; the conversion of —C(O)N₃ (which compound itself may be prepared from the corresponding acyl hydrazide under standard diazotisation reaction conditions, e.g. in the presence of NaNO₂ and a strong acid such as H₂SO₄ or HCl) to —NH₂, for example under Curtius rearrangement reaction conditions, which may result in the formation of an intermediate isocyanate (or a carbamate if treated with an alcohol); the conversion of an alkyl carbamate to —NH₂, by hydrolysis, for example in the presence of water and base or under acidic conditions, or, when a benzyl carbamate intermediate is formed, under hydrogenation reaction conditions (e.g. catalytic hydrogenation reaction conditions in the presence of a precious metal catalyst such as Pd); halogenation of an aromatic ring, for example by an electrophilic aromatic substitution reaction in the presence of halogen atoms (e.g. chlorine, bromine, etc, or an equivalent source thereof) and, if necessary an appropriate catalyst/Lewis acid (e.g. AlCl₃ or FeCl₃).

Further, the skilled person will appreciate that the D₁ to D₃-containing ring, as well as the A ring may be heterocycles, which moieties may be prepared with reference to a standard heterocyclic chemistry textbook (e.g. “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 or “Science of Synthesis”, Volumes 9-17 (Hetarenes and Related Ring Systems), Georg Thieme Verlag, 2006). Hence, the reactions disclosed herein that relate to compounds containing hetereocycles may also be performed with compounds that are pre-cursors to heterocycles, and which pre-cursors may be converted to those heterocycles at a later stage in the synthesis.

Compounds of the invention may be isolated (or purified) from their reaction mixtures using conventional techniques (e.g. crystallisations, recrystallisations or chromatographic techniques).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein. Protecting groups that may be mentioned include lactone protecting groups (or derivatives thereof), which may serve to protect both a hydroxy group and an α-carboxy group (i.e. such that the cyclic moiety is formed between the two functional groups.

The type of chemistry involved will dictate the need, and type, of protecting groups as well as the sequence for accomplishing the synthesis.

The use of protecting groups is described in e.g. “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention, including, but not limited to:
(a) compounds of formula I in which Y¹ represents —C(O)OR^{9a} in which R^{9a} is/are other than hydrogen, so forming an ester group; and/or
(b) compounds of formula I in which Y represents —C(═N—OR²⁹)—, i.e. the following compound of formula Ia,

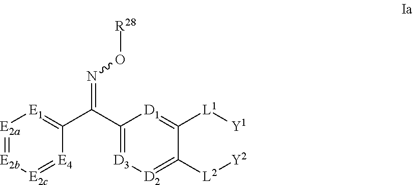

in which the integers are as hereinbefore defined (and the squiggly line indicates that the oxime may exist as a cis or trans isomer, as is apparent to the skilled person),
   may possess no or minimal pharmacological activity as such, but may be administered parenterally or orally, and thereafter be metabolised in the body to form compounds of the invention that possess pharmacological activity as such, including, but not limited to:
(A) corresponding compounds of formula I, in which Y¹ represents —C(O)OR^{9a} in which R^{9a} represent hydrogen (see (a) above); and/or
(B) corresponding compounds of formula I in which Y represents —C(O)—, for example in the case where the oxime or oxime ether of the compound of formula Ia (see (b) above) is hydrolysed to the corresponding carbonyl moiety.

Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the test described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III (preferably, MGST-II), thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTC₄ and/or LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulonephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined, in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of the invention, as hereinbefore defined, or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. leukotriene receptor antagonists (LTRas), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activting protein), immunosuppressants and sulphasalazine and related compounds and/or other therapeutic agents that are useful in the treatment of inflammation).

According to a further aspect of the invention, there is provided a combination product comprising:
(A) a compound of the invention, as hereinbefore defined; and
(B) another therapeutic agent that is useful in the treatment of a respiratory disorder and/or inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment of a respiratory disorder and/or inflammation, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Aqueous solubility is a fundamental molecular property that governs a large range of physical phenomena related to the specific chemical compound including e.g. environmental fate, human intestinal absorption, effectiveness of in vitro screening assays, and product qualities of water-soluble chemicals. By definition, the solubility of a compound is the maximum quantity of compound that can dissolve in a certain quantity of solvent at a specified temperature. Knowledge of a compound's aqueous solubility can lead to an understanding of its pharmacokinetics, as well as an appropriate means of formulation.

Compounds of the invention (especially those in which L² represents —C(O)-A¹⁷-, e.g. —C(O)—) may exhibit improved solubility properties (for instance compared to certain compounds disclosed in the prior art). Greater aqueous solubility (or greater aqueous thermodynamic solubility) may have advantages related to the effectiveness of the compounds of the invention (especially those in which L² represents —C(O)-A¹⁷-, e.g. —C(O)—), for instance improved absorption in vivo (e.g. in the human intestine) or the compounds may have other advantages associated with the physical phenomena related to improved aqueous stability (see above). Good (e.g. improved) aqueous solubility may aid the formulation of compounds of the invention, i.e. it may be easier and/or less expensive to manufacture tablets which will dissolve more readily in the stomach as potentially one can avoid esoteric and/or expensive additives and be less dependent on particle-size (e.g. micronization or grinding may be avoided) of the crystals, etc, and it may be easier to prepare formulations intended for intravenous administration.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### EXAMPLES

The invention is illustrated by way of the following examples, in which the following abbreviations may be employed:

### Abbreviations:

aq aqueous
BINAP 2,2′-bis(diphenylphosphino)-1,1′-binaphthyl
brine saturated aqueous solution of NaCl
DCM dichloromethane
DDQ 2,3-dicyano-5,6-dichloro-1,4-benzoquinone
DMAP N,N-dimethyl-4-aminopyridine
DMF dimethylformamide
DMSO dimethylsulfoxide
EtOAc ethyl acetate
EtOH ethanol
MeCN acetonitrile
MeOH methanol
NMR nuclear magnetic resonance
Oxone potassium peroxymonosulfate (2KHSO₅.KHSO₄.K₂SO₄)
Pd₂ dba₃ tris(dibenzylideneacetone)dipalladium(0)
dppf 1,1′-bis(diphenylphosphino)ferrocene
rt room temperature
rx reflux
sat saturated
THF tetrahydrofuran

### Biological Test

### Test 1

### In Vitro Assay

In the assay, LTC₄ synthase catalyses the reaction where the substrate LTA₄ is converted to LTC₄. Recombinant human LTC₄ synthase is expressed in Piccia pastoralis and the purified enzyme is dissolved in 25 mM tris-buffer pH 7.8 supplemented with 0.1 mM glutathione (GSH) and stored at −80° C. The assay is performed in phosphate buffered saline (PBS) pH 7.4 and 5 mM GSH in 384-well plates.

The following is added chronologically to each well:

1. 48 μL LTC₄ synthase in PBS with 5 mM GSH. The total protein concentration in this solution is 0.5 μg/mL.
2. 1 μL inhibitor in DMSO (final concentration 10 μM).
3. Incubation of the plate at room temperature for 10 min.
4. 1 μL LTA₄ (final concentration 2.5 μM).
5. Incubation of the plate at room temperature for 5 min.
6. 10 μL of the incubation mixture is analysed using homogenous time resolved fluorescent (HTRF) detection.

### Test 2

For certain compounds, in order to obtain IC₅₀-values for the compounds, following procedure was used:
1. A volume (24 μL) of 0.25 μg/mL LTC₄ synthase in 75 mM tris (pH ˜8.5), 0.5 mM MgCl₂ and 3 mM GSH was preincubated for 10 min with 0.5 μL of compound of interest in DMSO at ten different concentrations, typically in the range 10^{-9,5}-10⁻⁵M as well as with DMSO only. The buffer is used as background. Runs are performed in duplicates.
2. The enzymatic reaction is initiated by addition of 0.5 μL LTA₄ in diglyme (final assay concentration 8 μM).
3. The reaction is stopped after 1 min by addition of double the reaction volume of a stop solution (MeOH:H₂O:acetic acid 70:30:1).
4. After 15 fold dilution in PBS, LTC₄ formation is detected with LTC₄ HTRF kit (Cisbio, cat. No 64LC4PEC) and a fluorescence reader. Typically 10 μL of sample is mixed with 5 μL of each of the HTRF reagents d2 and k and analyzed as described in the kit manual.

The detected product concentration, with background subtracted, is plotted versus compound concentration and IC₅₀ is determined as 50% of maximum inhibition.

### Biological Examples

Title compounds of the Examples were tested in the biological in vitro assay described above (either Test 1 or Test 2 above) and were found to inhibit LTC₄ synthase. Title compounds of the examples exhibit a certain IC₅₀ value, which shows that they inhibit LTC₄ synthase. IC₅₀ values for title compounds of the examples are depicted in the tables hereinafter (where, they are the values obtained using Test 1, unless otherwise indicated).

In the event that there is a discrepancy between nomenclature and any compounds depicted graphically, then it is the latter that presides (unless contradicted by any experimental details that may be given or unless it is clear from the context).

The compounds of the examples (see hereinafter, and tables) may exist as a cyclised form, i.e. in a form depicted hereinbefore by compounds of formula I (whereby the compound depicted below may undergo an intramolecular cyclisation). Hence, the characterising data (e.g. NMR data) presented below may refer to the cyclised form of that compound. Alternatively, the compounds (of formula I; e.g. see the compounds of the examples depicted below) may exist in rapid or slow equilibrium (on an NMR time scale) with the cyclised form (of formula IA) and hence the spectra may represent either one of the compounds or both of the compounds (e.g. spectra for single compounds may be observed, or spectra for two compounds, which spectra may for instance overlap or merge).

### Example 1

### Example 1:1

### 2-Benzoyl-5-{5-[(4-chlorophenyl)(methyl)amino]picolinoyl}benzoic acid

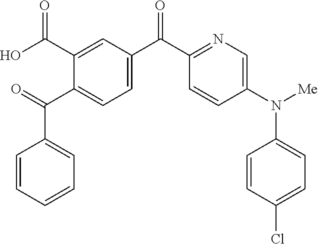 [SMILES: CN(c1ccc(Cl)cc1)c1ccc(C(=O)c2ccc(C(=O)c3ccccc3)c(C(=O)O)c2)nc1]

### (a) 2-Benzoyl-5-bromobenzoic acid methyl ester

The sub-title compound was obtained from 5-bromo-2-iodobenzoic acid methyl ester and benzoyl chloride in accordance with standard procedures. For instance, the iodo moiety may be converted to a Grignard reagent (e.g. by use of i-PrMgCl in THF) and the benzoyl chloride may then be added e.g. at low temperatures, such as below 0° C. (e.g. at −15° C. for 1 h) followed by standard work up (e.g. quenching with NH₄Cl (aq, sat) and extractive workup (EtOAc, H₂O, brine) and purification by chromatography).

### (b) 2-Benzoyl-5-iodobenzoic acid methyl ester

A mixture of 2-benzoyl-5-bromobenzoic acid methyl ester (5.207 g, 16.31 mmol), CuI (0.311 g, 1.63 mmol), NaI (4.889 g, 32.62 mmol), N¹,N²-dimethylethane-1,2-diamine (0.351 μL, 3.26 mmol) and dioxane (20 mL) was heated at 120° C. for 18 h. Extractive workup (EtOAc, NH₄Cl (aq, sat), H₂O, brine), drying (Na₂SO₄), concentration and purification by chromatography gave the sub-title compound. Yield: 4.633 g (78%).

### (c) 2-Benzoyl-5-[(5-bromo-2-pyridyl)hydroxymethyl]benzoic acid methyl ester

The sub-title compound was obtained from 2-benzoyl-5-iodobenzoic acid methyl ester and 5-bromopicolinaldehyde in accordance with the procedures described in step (a) above.

### (d) 2-Benzoyl-5-(5-bromopicolinoyl)benzoic acid methyl ester

Oxidation of 2-benzoyl-5-[(5-bromo-2-pyridyl)hydroxymethyl]benzoic acid methyl ester in accordance with standard procedures gave the sub-title compound. For instance, pyridinium chlorochromate (1 equivalent or slight excess) may added to the product in DCM at rt. After reaction, the mixture may be filtered through Celite, concentrated, treated with EtOAc:hexanes (1:2), filtered through silica and concentrated again.

### (e) 2-Benzoyl-5-{5-[(4-chlorophenyl)methylamino]picolinoyl}benzoic acid methyl ester

The title compound was prepared from 2-benzoyl-5-(5-bromopicolinoyl)benzoic acid methyl ester and 4-chloro-N-methylaniline in accordance with standard procedures. For instance, the compounds may be mixed/reacted with a catalyst system (e.g. Pd(OAc)₂, (catalytic amount), BINAP, base such as Cs₂CO₃ (e.g. at least one equivalent) and toluene and the reaction may be stirred for a period of time (e.g. at 80° C. for 20 h in a sealed tube). The mixture may be diluted with solvent (e.g. EtOAc) and filtered through Celite.

### (f) 2-Benzoyl-5-{5-[(4-chlorophenyl)(methyl)amino]picolinoyl}benzoic acid

The title compound was prepared from 2-benzoyl-5-{5-[(4-chlorophenyl)-methylamino]picolinoyl}benzoic acid methyl ester in accordance with standard procedures. For instance, NaOH in H₂O may be added to the compound dissolved in hot EtOH. The mixture may be heated at rx (e.g. for 30 min), cooled, concentrated and acidified with HCl to pH ˜2. This may be followed by extractive workup (EtOAc, H₂O, brine), drying (Na₂SO₄), concentration and purification by chromatography to give the title compound.

¹H NMR (DMSO-d₆) δ: 8.44-8.40 (1H, m) 8.20 (1H, d, J=2.8 Hz) 8.06-8.01 (1H, m) 7.97 (1H, d, J=8.9 Hz) 7.58-7.53 (2H, m) 7.53-7.46 (3H, m) 7.44-7.34 (4H, m) 7.31-7.23 (2H, m) 3.38 (3H, s). IC₅₀=72 nM.

### Examples 1:2-1:6

The title compounds were prepared in accordance with Example 1: 1 using the appropriate acid chloride in step 1:1 (a), the appropriate amine in step 1:1 (e), alkylation (when appropriate) in accordance with standard procedures (e.g. in the presence of base, such as NaH in an appropriate solvent, such as a polar aprotic solvent, and the alkylating reagent) followed by hydrolysis in accordance with Example 1: 1, step (e).

### Example 1:7

### 5-{5-[(4-Chlorophenyl)(methyl)amino]picolinoyl}-2-(3-methoxybenzoyl)benzoic acid

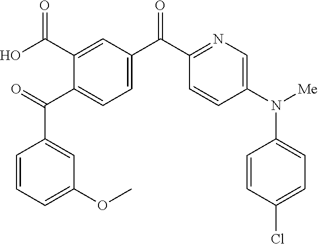 [SMILES: COc1cccc(C(=O)c2ccc(C(=O)c3ccc(N(C)c4ccc(Cl)cc4)cn3)cc2C(=O)O)c1]

### (a) 5-{5-[(4-Chlorophenyl)(methyl)amino]picolinoyl}-2-trimethylstannanylbenzoic acid methyl ester

A mixture of 2-bromo-5-{5-[(4-chlorophenyl)(methyl)amino]picolinoyl}benzoic acid methyl ester (100 mg, 0.217 mmol, see Example 2, step (c) hereinafter), 1,1,1,2,2,2-hexamethyldistannane (86 mg, 0.261 mmol), PdCl₂(PPh₃)₂ (5 mg, 0.0073 mmol) and toluene (15 mL) was stirred at 105° C. for 5 h. The mixture was cooled to rt, filtered through Celite, washed with EtOAc, concentrated and purified by chromatography to give the sub-title compound. Yield: 95 mg (80%).

### (b) 5-{5-[(4-Chlorophenyl)(methyl)amino]picolinoyl}-2-(3-methoxybenzoyl)-benzoic acid methyl ester

A mixture of 5-{5-[(4-chlorophenyl)(methyl)amino]picolinoyl}-2-trimethylstannanylbenzoic acid methyl ester (90 mg, 0.165 mmol), 3-methoxybenzoyl chloride (31 mg, 0.182 mmol), PdCl₂(PPh₃)₂ (2.2 mg, 0.0032 mmol) and toluene (1 mL) was stirred at 105° C. for 3 h. The mixture was cooled to rt and MeOH (2 mL) was added. The mixture was stirred at rt for 10 min, filtered through Celite, washed with EtOAc, concentrated and purified by chromatography to give the sub-title compound. Yield: 35 mg (40%).

### (c) 5-{5-[(4-Chlorophenyl)(methyl)amino]picolinoyl}-2-(3-methoxybenzoyl)-benzoic acid

The title compound was prepared from 5-{5-[(4-chlorophenyl)(methyl)amino]-picolinoyl}-2-(3-methoxybenzoyl)benzoic acid methyl ester in accordance with Example 1: 1, step (f). ¹H NMR (DMSO-d₆) δ: 8.54-8.43 (1H, m) 8.19 (1H, d, J=2.3 Hz) 8.10 (1H, d, J=6.7 Hz) 7.98 (1H, d, J=8.6 Hz) 7.55-7.45 (2H, m) 7.42-7.24 (5H, m) 7.22-7.15 (1H, m) 7.15-7.08 (1H, m) 7.04 (1H, d, J=7.4 Hz) 3.74 (3H, s) 3.38 (3H, s). IC₆₀=36 nM.

### Examples 1:8-1:20 and 1:24-1:29

The title compounds were prepared from 5-{5-[(4-chlorophenyl)(methyl)amino]-picolinoyl}-2-trimethylstannanylbenzoic acid methyl ester and the appropriate acid chloride in accordance with Example 1:7, steps (b) and (c), see Table 1. The palladium source in step (b) was allylpalladium(II) chloride dimer, Pd₂ dba₃, or Pd(P(t-Bu)₃)₂ with toluene or MeCN as solvent.

### Examples 1:21-1:23

The title compounds were prepared in accordance with Example 1:1 using the appropriate amine in step (e), followed by hydrolysis in accordance with Example 1:1, step (f).

TABLE 1

| | (in which the compounds may exist as the cyclised form; see above). | |
|---|---|---|
| | Chemical structure | IC₅₀ (nM) |
| | Name | |
| Ex. | ¹H-NMR (DMSO-d₆, δ) | |
| 1:2 | 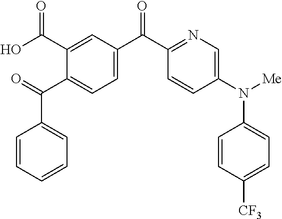 [SMILES: CN(c1ccc(C(F)(F)F)cc1)c1ccc(C(=O)c2ccc(C(=O)c3ccccc3)c(C(=O)O)c2)nc1] | 78 |
| | 2-Benzoyl-5-{5-[(methyl)(4-trifluoromethylphenyl)amino] | |
| | picolinoyl}benzoic acid | |
| | 8.45-8.39 (2H, m) 8.03-7.95 (2H, m) 7.75-7.68 (2H, m) 7.60-7.50 | |
| | (3H, m) 7.50-7.34 (5H, m) 7.19 (1H, d, J = 7.6 Hz) 3.45 (3H, s) | |
| 1:3 | 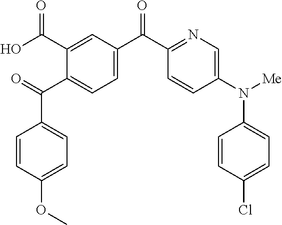 [SMILES: COc1ccc(C(=O)c2ccc(C(=O)c3ccc(N(C)c4ccc(Cl)cc4)cn3)cc2C(=O)O)cc1] | 50 |
| | 5-{5-[(4-Chlorophenyl)(methyl)amino]picolinoyl}-2- | |
| | (4-methoxybenzoyl)benzoic acid | |
| | 13.4-13.2 (1H, br s) 8.52 (1H, d, J = 1.6 Hz) 8.24 (1H, dd, J = 7.9, | |
| | 1.6 Hz) 8.19 (1H, d, J = 2.8 Hz) 8.01 (1H, d, J = 8.9 Hz) 7.63-7.57 | |
| | (2H, m) 7.53-7.45 (3H, m) 7.39-7.33 (2H, m) 7.28 (1H, dd, J = 8.9, | |
| | 2.8 Hz) 7.04-6.98 (2H, m) 3.80 (3H, s) 3.38 (3H, s) | |
| 1:4 | 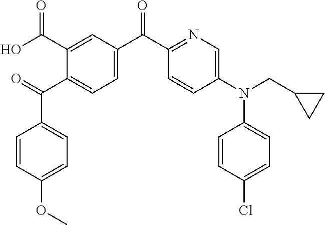 [SMILES: COc1ccc(C(=O)c2ccc(C(=O)c3ccc(N(CC4CC4)c4ccc(Cl)cc4)cn3)cc2C(=O)O)cc1] | 29 |
| | 5-{5-[(4-Chlorophenyl)(cyclopropylmethyl)amino]picolinoyl}- | |
| | 2-(4-methoxy-benzoyl)benzoic acid | |
| | 13.3-13.0 (1H, br s) 8.39 (1H, d, J = 1.6 Hz) 8.12 (1H, dd, | |
| | J = 7.9, 1.6 Hz) 8.01 (1H, d, J = 2.8 Hz) 7.89 (1H, d, J = 8.9 Hz) | |
| | 7.51-7.45 (2H, m) 7.44-7.38 (2H, m) 7.35 (1H, d, J = 7.9 Hz) | |
| | 7.28-7.22 (2H, m) 7.12 (1H, dd, J = 8.9, 2.8 Hz) 6.92-6.87 (2H, m) | |
| | 3.69 (3H, s) 3.58 (2H, d, J = 6.5 Hz) 1.01-0.90 (1H, m) 0.34-0.26 | |
| | (2H, m) 0.04-(−0.01) (2H, m) | |
| 1:5 | 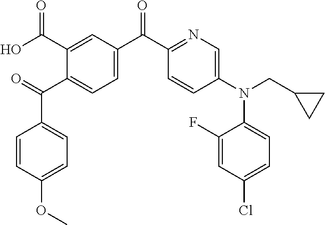 [SMILES: COc1ccc(C(=O)c2ccc(C(=O)c3ccc(N(CC4CC4)c4ccc(Cl)cc4F)cn3)cc2C(=O)O)cc1] | 46 |
| | 5-{5-[(4-Chloro-2-fluorophenyl)(cyclopropylmethyl)amino] | |
| | picolinoyl}-2-(4-methoxybenzoyl)benzoic acid | |
| | 13.2-13.1 (1H, br s) 8.41 (1H, d, J = 1.1 Hz) 8.14 (1H, dd, J = 7.8, | |
| | 1.1 Hz) 7.99 (1H, d, J = 2.5 Hz) 7.92 (1H, d, J = 8.9 Hz) 7.58-7.41 | |
| | (4H, m) 7.39-7.30 (2H, m) 7.08 (1H, dd, J = 8.9, 2.5 Hz) 6.94-6.88 | |
| | (2H, m) 3.70 (3H, s) 3.57 (2H, d, J = 6.5 Hz) 0.99-0.88 (1H, m) | |
| | 0.35-0.25 (2H, m) 0.03-(−0.02) (2H, m) | |
| 1:6 | 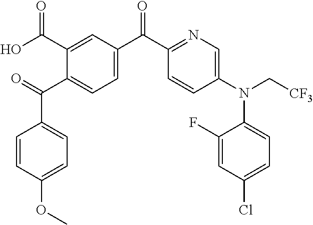 [SMILES: COc1ccc(C(=O)c2ccc(C(=O)c3ccc(N(CC(F)(F)F)c4ccc(Cl)cc4F)cn3)cc2C(=O)O)cc1] | 401 |
| | 5-{5-[(4-Chloro-2-fluorophenyl)(2,2,2-trifluoroethyl)amino] | |
| | picolinoyl}-2-(4-methoxybenzoyl)benzoic acid | |
| | 8.54-8.50 (1H, m) 8.29-8.20 (2H, m) 8.05 (1H, d, J = 8.9 Hz) | |
| | 7.75-7.55 (4H, m) 7.54-7.44 (2H, m) 7.36-7.24 (1H, m) 7.07-6.97 | |
| | (2H, m) 4.83 (2H, q, J = 8.7 Hz) 3.81 (3H, s) | |
| 1:8 | 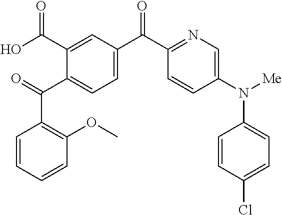 [SMILES: COc1ccccc1C(=O)c1ccc(C(=O)c2ccc(N(C)c3ccc(Cl)cc3)cn2)cc1C(=O)O] | 154 |
| | 5-{5-[(4-Chlorophenyl)(methyl)amino]picolinoyl}-2- | |
| | (2-methoxybenzoyl)benzoic acid | |
| | 8.49-8.37 (1H, m) 8.19 (1H, d, J = 2.3 Hz) 8.10 (1H, d, J = 7.0 Hz) | |
| | 7.97 (1H, d, J = 9.0 Hz) 7.66 (1H, d, J = 7.0 Hz) 7.54-7.45 (3H, m) | |
| | 7.38-7.33 (2H, m) 7.31-7.23 (2H, m) 7.06-6.97 (2H, m) 3.46 (3H, s) | |
| | 3.38 (3H, s) | |
| 1:9 | 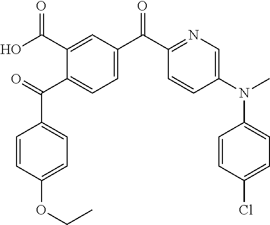 [SMILES: CCOc1ccc(C(=O)c2ccc(C(=O)c3ccc(N(C)c4ccc(Cl)cc4)cn3)cc2C(=O)O)cc1] | 101 |
| | 5-{5-[(4-Chlorophenyl)(methyl)amino]picolinoyl}-2- | |
| | (4-ethoxybenzoyl)benzoic acid | |
| | 13.8-13.1 (1H, br s) 8.54-8.44 (1H, m) 8.19 (1H, d, J = 2.3 Hz) 8.13 | |
| | (1H, d, J = 7.0 Hz) 7.99 (1H, d, J = 9.0 Hz) 7.60-7.45 (4H, m) | |
| | 7.41-7.31 (3H, m) 7.28 (1H, dd, J = 9.0, 2.7 Hz) 7.00-6.91 (2H, m) | |
| | 4.06 (2H, q, J = 7.0 Hz) 3.38 (3H, s) 1.30 (3H, t, J = 7.0 Hz) | |
| 1:10 | 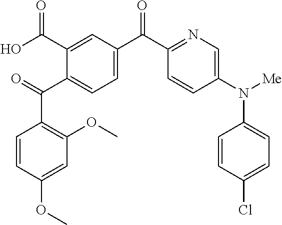 [SMILES: COc1ccc(C(=O)c2ccc(C(=O)c3ccc(N(C)c4ccc(Cl)cc4)cn3)cc2C(=O)O)c(OC)c1] | 68 |
| | 5-{5-[(4-Chlorophenyl)(methyl)amino]picolinoyl}-2- | |
| | (2,4-dimethoxybenzoyl)-benzoic acid | |
| | 8.50-8.37 (1H, m) 8.18 (1H, d, J = 2.3 Hz) 8.05 (1H, d, J = 7.0 Hz) | |
| | 7.95 (1H, d, J = 9.0 Hz) 7.61 (1H, d, J = 9.0 Hz) 7.55-7.44 (2H, m) | |
| | 7.39-7.33 (2H, m) 7.28 (1H, dd, J = 9.0, 2.7 Hz) 7.16 (1H, d, J = | |
| | 7.0 Hz) 6.56 (1H, d, J = 9.0 Hz) 6.53-6.48 (1H, m) 3.79 (3H, s) | |
| | 3.45 (3H, s) 3.37 (3H, s) | |
| 1:11 | 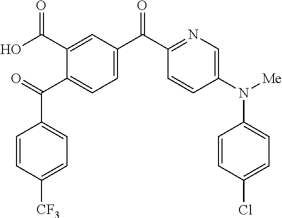 [SMILES: CN(c1ccc(Cl)cc1)c1ccc(C(=O)c2ccc(C(=O)c3ccc(C(F)(F)F)cc3)c(C(=O)O)c2)nc1] | 216 |
| | 5-{5-[(4-Chlorophenyl)(methyl)amino]picolinoyl}-2- | |
| | (4-trifluoromethylbenzoyl)-benzoic acid | |
| | 13.8-13.2 (1H, br s) 8.58-8.46 (1H, m) 8.23 (1H, d, J = 7.0 Hz) | |
| | 8.20 (1H, d, J = 2.7 Hz) 8.01 (1H, d, J = 9.0 Hz) 7.93-7.81 | |
| | (2H, m) 7.81-7.70 (2H, m) 7.65-7.42 (3H, m) 7.41-7.33 (2H, m) | |
| | 7.29 (1H, dd, J = 9.0, 2.7 Hz) 3.39 (3H, s) | |
| 1:12 | 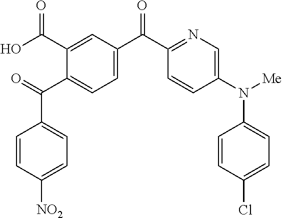 [SMILES: CN(c1ccc(Cl)cc1)c1ccc(C(=O)c2ccc(C(=O)c3ccc([N+](=O)[O-])cc3)c(C(=O)O)c2)nc1] | 202 |
| | 5-{5-[(4-Chlorophenyl)(methyl)amino]picolinoyl}-2- | |
| | (4-nitrobenzoyl)benzoic acid | |
| | 8.56-8.46 (1H, m) 8.32-8.23 (2H, m) 8.23-8.13 (2H, m) 8.00 | |
| | (1H, d, J = 9.0 Hz) 7.86-7.73 (2H, m) 7.58-7.43 (3H, m) 7.40-7.33 | |
| | (2H, m) 7.28 (1H, dd, J = 9.0, 2.7 Hz) 3.38 (3H, s) | |
| 1:13 | 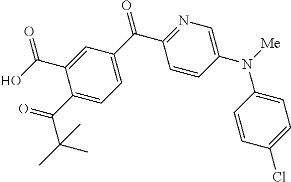 [SMILES: CN(c1ccc(Cl)cc1)c1ccc(C(=O)c2ccc(C(=O)C(C)(C)C)c(C(=O)O)c2)nc1] | 491 |
| | 5-{5-[(4-Chlorophenyl)(methyl)amino]picolinoyl}-2- | |
| | (2,2-dimethylpropionyl)-benzoic acid | |
| | 8.4-8.3 (1H, br s) 8.26 (1H, d, J = 7.0 Hz) 8.19 (1H, d, J = 2.7 Hz) | |
| | 8.00 (1H, d, J = 9.0 Hz) 7.95-7.83 (1H, m) 7.83-7.66 (1H, m) | |
| | 7.56-7.45 (2H, m) 7.40-7.32 (2H, m) 7.27 (1H, dd, J = 9.0, 2.7 Hz) | |
| | 3.38 (3H, s) 0.98 (9H, s) | |
| 1:14 | 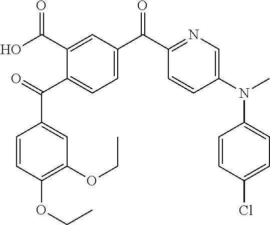 [SMILES: CCOc1ccc(C(=O)c2ccc(C(=O)c3ccc(N(C)c4ccc(Cl)cc4)cn3)cc2C(=O)O)cc1OCC] | 77 |
| | 5-{5-[(4-Chlorophenyl)(methyl)amino]picolinoyl}-2- | |
| | (3,4-diethoxybenzoyl)-benzoic acid | |
| | 8.52-8.44 (1H, m) 8.19 (1H, d, J = 2.7 Hz) 8.16-8.08 (1H, m) | |
| | 7.98 (1H, d, J = 9.0 Hz) 7.54-7.46 (2H, m) 7.41-7.31 (4H, m) 7.28 | |
| | (1H, dd, J = 9.0, 2.7 Hz) 6.98-6.90 (2H, m) 4.04 (2H, q, J = 7.0 | |
| | Hz) 4.01 (2H, q, J = 7.0 Hz) 3.38 (3H, s) 1.30 (3H, t, J = 7.0 Hz) | |
| | 1.30 (3H, t, J = 7.0 Hz) | |
| 1:15 | 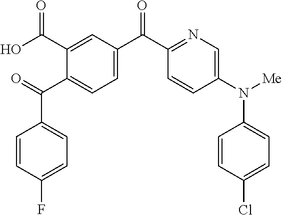 [SMILES: CN(c1ccc(Cl)cc1)c1ccc(C(=O)c2ccc(C(=O)c3ccc(F)cc3)c(C(=O)O)c2)nc1] | 126 |
| | 5-{5-[(4-Chlorophenyl)(methyl)amino]picolinoyl}-2- | |
| | (4-fluorobenzoyl)benzoic acid | |
| | 8.56-8.44 (1H, m) 8.19 (1H, d, J = 2.7 Hz) 8.17-8.09 (1H, m) | |
| | 7.99 (1H, d, J = 9.0 Hz) 7.72-7.60 (2H, m) 7.55-7.46 (2H, m) | |
| | 7.43-7.33 (3H, m) 7.32-7.22 (3H, m) 3.38 (3H, s) | |
| 1:16 | 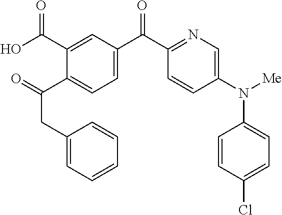 [SMILES: CN(c1ccc(Cl)cc1)c1ccc(C(=O)c2ccc(C(=O)Cc3ccccc3)c(C(=O)O)c2)nc1] | 257 |
| | 5-{5-[(4-Chlorophenyl)(methyl)amino]picolinoyl}-2- | |
| | phenylacetylbenzoic acid | |
| | 8.29-8.05 (2H, m) 8.17 (1H, d, J = 2.7 Hz) 7.97 (1H, d, J = 9.0 | |
| | Hz) 7.70-7.41 (1H, m) 7.53-7.47 (2H, m) 7.38-7.32 (2H, m) | |
| | 7.26 (1H, dd, J = 9.0, 2.7 Hz) 7.31-6.95 (5H, m) 3.62-3.32 (2H, m) | |
| | 3.37 (3H, s) | |
| 1:17 | 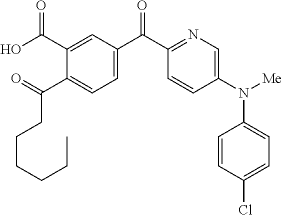 [SMILES: CCCCCCC(=O)c1ccc(C(=O)c2ccc(N(C)c3ccc(Cl)cc3)cn2)cc1C(=O)O] | 90 |
| | 5-{5-[(4-Chlorophenyl)(methyl)amino]picolinoyl}-2- | |
| | heptanoylbenzoic acid | |
| | 8.36-8.29 (1H, m) 8.26-8.19 (1H, m) 8.17 (1H, d, J = 2.7 Hz) 7.99 | |
| | (1H, d, J = 9.0 Hz) 7.72-7.54 (1H, m) 7.53-7.47 (2H, m) 7.38-7.33 | |
| | (2H, m) 7.27 (1H, dd, J = 9.0, 2.7 Hz) 3.38 (3H, s) 1.56-1.07 | |
| | (10H, m) 0.80 (3H, t, J = 6.7 Hz) | |
| 1:18 | 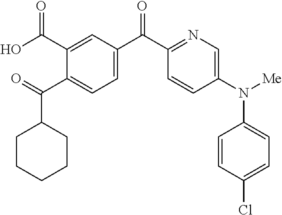 [SMILES: CN(c1ccc(Cl)cc1)c1ccc(C(=O)c2ccc(C(=O)C3CCCCC3)c(C(=O)O)c2)nc1] | 246 |
| | 5-{5-[(4-Chlorophenyl)(methyl)amino]picolinoyl}-2- | |
| | cyclohexylcarbonylbenzoic acid | |
| | 8.40-8.29 (1H, m) 8.29-8.20 (1H, m) 8.18 (1H, d, J = 2.7 Hz) 8.00 | |
| | (1H, d, J = 9.0 Hz) 7.80-7.54 (1H, m) 7.54-7.47 (2H, m) 7.40-7.32 | |
| | (2H, m) 7.27 (1H, dd, J = 9.0, 2.7 Hz) 3.38 (3H, s) 2.10-2.73 | |
| | (2H, m) 1.71-1.63 (2H, m) 1.60-1.55 (1H, m) 1.36-0.70 (6H, m) | |
| 1:19 | 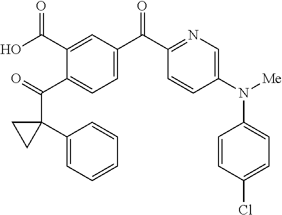 [SMILES: CN(c1ccc(Cl)cc1)c1ccc(C(=O)c2ccc(C(=O)C3(c4ccccc4)CC3)c(C(=O)O)c2)nc1] | 32 |
| | 5-{5-[(4-Chlorophenyl)(methyl)amino]picolinoyl}-2- | |
| | (1-phenylcyclopropyl-carbonyl)benzoic acid | |
| | 8.33-8.20 (1H, m) 8.14 (1H, d, J = 2.7 Hz) 8.07 (1H, d, J = 7.4 Hz) | |
| | 7.92 (1H, d, J = 9.0 Hz) 7.56-7.45 (2H, m) 7.42-7.31 (3H, m) 7.30- | |
| | 7.26 (2H, m) 7.24 (1H, dd, J = 9.0, 2.7 Hz) 7.15-7.03 (3H, m) 3.36 | |
| | (3H, s) 1.65-1.53 (2H, m) 1.34-1.20 (2H, m) | |
| 1:20 | 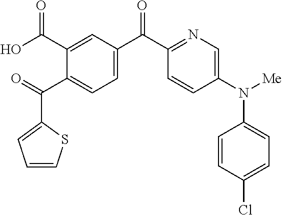 [SMILES: CN(c1ccc(Cl)cc1)c1ccc(C(=O)c2ccc(C(=O)c3cccs3)c(C(=O)O)c2)nc1] | 104 |
| | 5-{5-[(4-Chlorophenyl)(methyl)amino]picolinoyl}-2- | |
| | (2-thienylcarbonyl)benzoic acid | |
| | 8.51-8.41 (1H, m) 8.18 (1H, d, J = 2.7 Hz) 8.06 (1H, d, J = 7.4 Hz) | |
| | 7.97 (1H, d, J = 9.0 Hz) 7.91 (1H, d, J = 4.3 Hz) 7.54-7.47 (2H, m) | |
| | 7.42-7.32 (3H, m) 7.28 (1H, dd, J = 9.0, 2.7 Hz) 7.15 (1H, d, J = 2.7 | |
| | Hz) 7.12-7.01 (1H, m) 3.38 (3H, s) | |
| 1:21 | 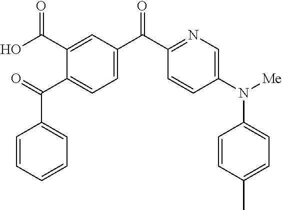 [SMILES: Cc1ccc(N(C)c2ccc(C(=O)c3ccc(C(=O)c4ccccc4)c(C(=O)O)c3)nc2)cc1] | 250 |
| | 2-Benzoyl-5-{[5-(methyl)(4-methylphenyl)amino]picolinoyl} | |
| | benzoic acid | |
| | 13.5-13.2 (1H, br s) 8.55 (1H, d, J = 1.5 Hz) 8.27 (1H, dd, J = 7.8, | |
| | 1.5 Hz) 8.14 (1H, d, J = 2.9 Hz) 8.03 (1H, d, J = 9.0 Hz) 7.68-7.59 | |
| | (3H, m) 7.54-7.48 (3H, m) 7.33-7.29 (2H, m) 7.26-7.22 (2H, m) 7.20 | |
| | (1H, dd, J = 9.0, 2.9 Hz) 3.39 (3H, s) 3.35 (3H, s) | |
| 1:22 | 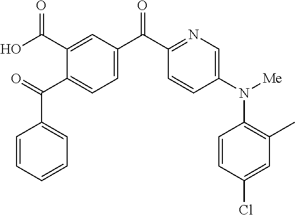 [SMILES: Cc1cc(Cl)ccc1N(C)c1ccc(C(=O)c2ccc(C(=O)c3ccccc3)c(C(=O)O)c2)nc1] | 442 |
| | 2-Benzoyl-5-{5-[(4-chloro-2-methylphenyl)(methyl)amino] | |
| | picolinoyl}benzoic acid | |
| | 13.6-13.3 (1H, br s) 8.54 (1H, d, J = 1.5 Hz) 8.26 (1H, dd, J = 8.0, | |
| | 1.5 Hz) 8.03 (1H, d, J = 9.0 Hz) 8.00-7.94 (1H, m) 7.67-7.59 | |
| | (3H, m) 7.55-7.47 (4H, m) 7.42 (1H, dd, J = 8.5, 2.5 Hz) 7.32 | |
| | (1H, d, J = 8.5 Hz) 7.01-6.63 (1H, m) 3.34 (3H, s) 2.11 (3H, s) | |
| 1:23 | 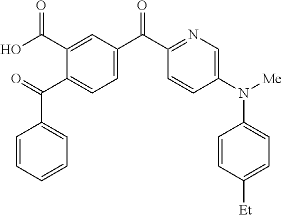 [SMILES: CCc1ccc(N(C)c2ccc(C(=O)c3ccc(C(=O)c4ccccc4)c(C(=O)O)c3)nc2)cc1] | 184 |
| | 2-Benzoyl-5-{5-[(4-ethylphenyl)(methyl)amino]picolinoyl} | |
| | benzoic acid | |
| | 13.6-13.2 (1H, br s) 8.54 (1H, d, J = 1.5 Hz) 8.25 (1H, dd, J = 8.0, | |
| | 1.5 Hz) 8.13 (1H, d, J = 3.0 Hz) 8.02 (1H, d, J = 9.0 Hz) 7.68-7.58 | |
| | (3H, m) 7.53-7.46 (3H, m) 7.36-7.31 (2H, m) 7.28-7.24 (2H, m) | |
| | 7.20 (1H, dd, J = 9.0, 3.0 Hz) 3.39 (3H, s) 2.64 (2H, q, J = 7.7 Hz) | |
| | 1.20 (3H, t, J = 7.7 Hz) | |
| 1:24 | 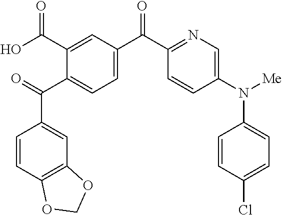 [SMILES: CN(c1ccc(Cl)cc1)c1ccc(C(=O)c2ccc(C(=O)c3ccc4c(c3)OCO4)c(C(=O)O)c2)nc1] | 77 |
| | 5-{5-[(4-Chlorophenyl)(methyl)amino]picolinoyl}-2- | |
| | (3,4-methylenedioxy-benzoyl)benzoic acid | |
| | 8.52-8.42 (1H, m) 8.19 (1H, d, J = 2.7 Hz) 8.09 (1H, d, J = 6.7 | |
| | Hz) 7.98 (1H, d, J = 9.0 Hz) 7.53-7.49 (2H, m) 7.39-7.34 (2H, | |
| | m) 7.34-7.27 (2H, m) 7.22-7.10 (1H, m) 7.01 (1H, dd, J = 8.2, | |
| | 1.6 Hz) 6.91 (1H, d, J = 8.2 Hz) 6.09 (2H, s) 3.38 (3H, s) | |
| 1:25 | 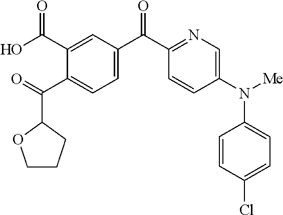 [SMILES: CN(c1ccc(Cl)cc1)c1ccc(C(=O)c2ccc(C(=O)C3CCCO3)c(C(=O)O)c2)nc1] | 860 |
| | 5-{5-[(4-Chlorophenyl)(methyl)amino]picolinoyl}- | |
| | 2-tetrahydrofuran-2-carbonylbenzoic acid | |
| | 8.35-8.26 (1H, m) 8.25 (1H, d, J = 8.2 Hz) 8.22-8.12 (2H, m) 8.01 | |
| | (1H, d, J = 9.0 Hz) 7.84-7.70 (1H, m) 7.58-7.45 (2H, m) 7.42-7.33 | |
| | (2H, m) 7.28 (1H, dd, J = 9.0, 2.7 Hz) 4.43-4.05 (1H, m) 3.68-3.47 | |
| | (2H, m) 3.38 (3H, s) 2.16-1.94 (2H, m) 1.88-1.67 (2H, m) | |
| 1:26 | 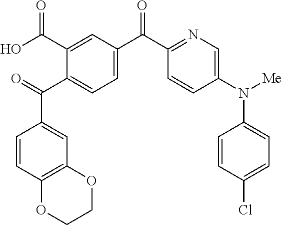 [SMILES: CN(c1ccc(Cl)cc1)c1ccc(C(=O)c2ccc(C(=O)c3ccc4c(c3)OCCO4)c(C(=O)O)c2)nc1] | 27 |
| | 5-{5-[(4-Chlorophenyl)(methyl)amino]picolinoyl- | |
| | 2-(3,4-ethylenedioxybenzoyl)-benzoic acid | |
| | 13.7-13.2 (1H, br s) 8.54-8.44 (1H, m) 8.20 (1H, d, J = 2.7 Hz) | |
| | 8.14 (1H, d, J = 6.7 Hz) 7.99 (1H, d, J = 9.0 Hz) 7.55-7.46 (2H, m) | |
| | 7.42-7.32 (3H, m) 7.28 (1H, dd, J = 9.0, 2.7 Hz) 7.11-7.01 (2H, m) | |
| | 6.90 (1H, d, J = 9.0 Hz) 4.30-4.26 (2H, m) 4.26-4.21 (2H, m) 3.38 | |
| | (3H, s) | |
| 1:27 | 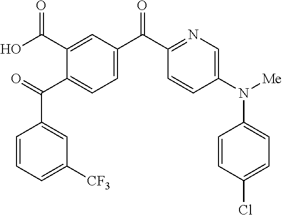 [SMILES: CN(c1ccc(Cl)cc1)c1ccc(C(=O)c2ccc(C(=O)c3cccc(C(F)(F)F)c3)c(C(=O)O)c2)nc1] | 54 |
| | 5-{5-[(4-Chlorophenyl)(methyl)amino]picolinoyl-2- (3-trifluoromethylbenzoyl)-benzoic acid | |
| | 13.7-13.3 (1H, br s) 8.59-8.48 (1H, m) 8.27 (1H, d, J = 7.8 Hz) | |
| | 8.20 (1H, d, J = 2.7 Hz) 8.02 (1H, d, J = 9.0 Hz) 7.98 (1H, d, | |
| | J = 7.8 Hz) 7.95-7.88 (1H, m) 7.81 (1H, d, J = 7.8 Hz) 7.76-7.68 | |
| | (1H, m) 7.57 (1H, d, J = 7.8 Hz) 7.54-7.46 (2H, m) 7.40-7.33 | |
| | (2H, m) 7.28 (1H, dd, J = 9.0, 2.7 Hz) 3.38 (3H, s) | |
| 1:28 | 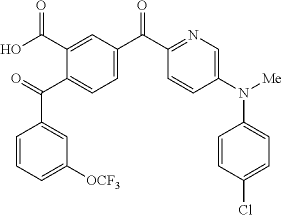 [SMILES: CN(c1ccc(Cl)cc1)c1ccc(C(=O)c2ccc(C(=O)c3cccc(OC(F)(F)F)c3)c(C(=O)O)c2)nc1] | 60 |
| | 5-{5-[(4-Chlorophenyl)(methyl)amino]picolinoyl-2- | |
| | (3-trifluoromethoxybenzoyl)-benzoic acid | |
| | 8.54-8.42 (1H, m) 8.19 (1H, d, J = 2.7 Hz) 8.11 (1H, d, J = 7.0 Hz) | |
| | 7.98 (1H, d, J = 9.0 Hz) 7.61-7.52 (3H, m) 7.52-7.48 (2H, m) 7.47- | |
| | 7.43 (1H, m) 7.42-7.32 (3H, m) 7.29 (1H, dd, J = 9.0, 2.7 Hz) 3.38 | |
| | (3H, s) | |
| 1:29 | 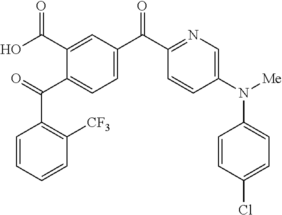 [SMILES: CN(c1ccc(Cl)cc1)c1ccc(C(=O)c2ccc(C(=O)c3ccccc3C(F)(F)F)c(C(=O)O)c2)nc1] | 245 |

5-{5-[(4-Chlorophenyl)(methyl)amino]picolinoyl-2-
(2-trifluoromethylbenzoyl)-benzoic acid
8.44-8.31 (1H, m) 8.17 (1H, d, J = 2.7 Hz) 8.07-7.94 (1H, m) 7.97
(1H, d, J = 9.0 Hz) 7.87 (1H, d, J = 7.8 Hz) 7.71-7.64 (1H, m) 7.64-
7.58 (1H, m) 7.52-7.48 (2H, m) 7.43 (1H, d, J = 7.8 Hz) 7.38-7.34
(2H, m) 7.33-7.21 (1H, m) 7.28 (1H, dd, J = 9.0, 2.7 Hz) 3.37 (3H, s)

### Example 2

### 5-(5-((4-Chlorophenyl)(methyl)amino)picolinoyl)-2-(3-(dichloromethyl)benzoyl)-benzoic acid

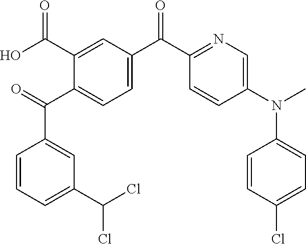 [SMILES: CN(c1ccc(Cl)cc1)c1ccc(C(=O)c2ccc(C(=O)c3cccc(C(Cl)Cl)c3)c(C(=O)O)c2)nc1]

### (a) 5-((4-Chlorophenyl)(methyl)amino)picolinaldehyde

4-Chloro-N-methylaniline (1.6 mL, 12.9 mmol) in toluene (44 mL) was added to a mixture of Cs₂CO₃ (4.9 g, 15.05 mmol), Pd(OAc)₂ (0.121 g, 0.538 mmol), BINAP (0.502 g, 0.806 mmol) and 5-bromopicolinaldehyde (2.00 g, 10.75 mmol). The mixture was stirred at 85° C. for 15 h and after cooling filtered through Celite. The solids were washed with EtOAc. The combined filtrates were concentrated and the residue purified by chromatography to give the sub-title compound. Yield: 1.537 g (58%).

### (b) 2-Bromo-5-((5-((4-chlorophenyl)(methyl)amino)pyridin-2-yl)(hydroxy)-methyl)benzoic acid methyl ester

i-PrMgCl (1.54 mL, 3.08 mmol, 2 M in THF) was added to 2-bromo-5-iodobenzoic acid methyl ester (1.0 g, 2.93 mmol) in THF (10 mL) at −15° C. The mixture was cooled to −45° C. and a cold (−45° C.) solution of 5-((4-chlorophenyl)(methyl)-amino)picolinaldehyde (0.80 g, 3.23 mmol) in THF (20 mL) was added. The mixture was allowed to reach rt and stirred for 16 h. NH₄Cl (aq, sat, 30 mL) was added at 0° C. and the mixture was stirred at rt for 30 min. Extractive workup (EtOAc, water, brine), drying (Na₂SO₄) and purification by chromatography gave the sub-title compound. Yield: 1.1 g (73%).

### (c) 2-Bromo-5-(5((4-chlorophenyl)(methyl)amino)picolinoyl)benzoic acid methyl ester

A mixture of 2-bromo-5-((5-((4-chlorophenyl)(methyl)amino)pyridin-2-yl)(hydroxy)-methyl)benzoic acid methyl ester (10.65 g, 23.06 mmol), DDQ (6.28 g, 27.68 mmol) and dioxane (75 mL) was stirred at rt for 1.5 h. The mixture was filtered through celite and the solids washed with EtOAc. The combined filtrates were concentrated and the residue purified by chromatography to give the sub-title compound. Yield: 6.23 g (58%).

### (d) 5-(5-((4-Chlorophenyl)(methyl)amino)picolinoyl)-2-trimethylstannanylbenzoic acid methyl ester

A mixture of 2-bromo-5-(5-((4-chlorophenyl)(methyl)amino)picolinoyl)benzoic acid methyl ester (100 mg, 0.217 mmol), 1,1,1,2,2,2-hexamethyldistannane (86 mg, 0.261 mmol), PdCl₂(PPh₃)₂ (5 mg, 0.0073 mmol) and toluene (15 mL) was stirred at 105° C. for 5 h. The mixture was cooled to rt, filtered through Celite, washed with EtOAc, concentrated and purified by chromatography to give the sub-title compound. Yield: 95 mg (80%).

### (e) 5-(5-((4-Chlorophenyl)(methyl)amino)picolinoyl)-2-(3-dichloromethyl-benzoyl)benzoic methyl ester

A mixture of 3-dichloromethylbenzoyl chloride (90 mg, 0.405 mmol), allylpalladium chloride dimer (6.6 mg) and toluene (0.5 mL) was added to 5-(5-((4-chlorophenyl)(methyl)amino)picolinoyl)-2-trimethylstannanylbenzoic acid methyl ester (200 mg, 0.368 mmol) in toluene (1.5 mL) at rt. The mixture was heated at 44° C. for 4.5 h and cooled to rt. Extractive workup (EtOAc, water, brine), drying (Na₂SO₄) and purification by chromatography gave the sub-title compound. Yield: 64 mg (30%).

### (f) 5-(5-((4-Chlorophenyl)(methyl)amino)picolinoyl)-2-(3-dichloromethyl-benzoyl)benzoic acid

A mixture of 5-(5-((4-chlorophenyl)(methyl)amino)picolinoyl)-2-(3-dichloromethylbenzoyl)benzoic methyl ester (54 mg, 0.095 mmol), EtOH (30 mL), water (6 mL) and NaOH (19 mg, 0.475 mmol) was stirred at 65° C. for 5 min. The pH was adjusted to ˜5 with HCl (aq, 1M). Concentration, extractive workup (EtOAc, water, brine), drying (Na₂SO₄) and purification by chromatography gave the title compound. Yield: 50 mg (95%). ¹H NMR (DMSO-d₆) δ: 8.54-8.46 (1H, m) 8.20 (1H, d, J=2.7 Hz) 8.18-8.09 (1H, m) 7.99 (1H, d, J=9.0 Hz) 7.96-7.91 (1H, m) 7.86-7.78 (1H, m) 7.56 (1H, s) 7.55-7.46 (4H, m) 7.46-7.33 (3H, m) 7.29 (1H, dd, J=9.0, 2.7 Hz) 3.38 (3H, s). IC₅₀=34 nM.

### Example 3

### 5-(5-((4-Chlorophenyl)(methyl)amino)picolinoyl)-2-(3-formylbenzoyl)benzoic acid

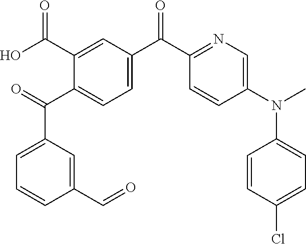 [SMILES: CN(c1ccc(Cl)cc1)c1ccc(C(=O)c2ccc(C(=O)c3cccc(C=O)c3)c(C(=O)O)c2)nc1]

A mixture of 5-(5-((4-chlorophenyl)(methyl)amino)picolinoyl)-2-(3-(dichloro-methyl)benzoyl)benzoic acid (20 mg, 0.036 mmol), NaOH (0.3 mL, 1M (aq)), water (2.5 mL), dimethylamine hydrochloride (1.0 g, 0.012 mmol) and EtOH (2 mL) was heated at 60° C. for 50 h. After cooling to rt, the pH was adjusted to ˜5 with HCl (aq, 1M). Concentration, extractive workup (EtOAc, water, brine), drying (Na₂SO₄) and purification by chromatography gave the title compound. Yield: 15 mg (83%). ¹H NMR (DMSO-d₆) δ: 10.0 (1H, s) 8.54-8.45 (1H, m) 8.20 (1H, d, J=2.7 Hz) 8.16-8.09 (1H, m) 8.08-8.02 (2H, m) 7.99 (1H, d, J=9.0 Hz) 7.92-7.83 (1H, m) 7.72-7.63 (1H, m) 7.52-7.48 (2H, m) 7.42-7.32 (3H, m) 7.29 (1H, dd, J=9.0, 2.7 Hz) 3.38 (3H, s). IC₅₀=52 nM.

### Example 4

### 5-(5-((4-Chlorophenyl)(cyclopropylmethyl)amino)picolinoyl)-2-(2,3-dihydrobenzo-[b][1,4]dioxine-6-carbonyl)benzoic acid

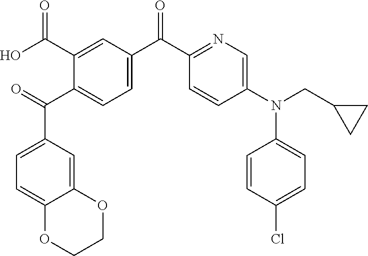 [SMILES: O=C(c1ccc(C(=O)c2ccc3c(c2)OCCO3)c(C(=O)O)c1)c1ccc(N(CC2CC2)c2ccc(Cl)cc2)cn1]

### (a) 5((4-Chlorophenyl)amino)picolinaldehyde

The sub-title compound was prepared in accordance with example 2 step (a) using 4-chloroaniline.

### (b) 5((4-Chlorophenyl)(cyclopropylmethyl)amino)picolinaldehyde

Sodium hydride (0.68 g, 17.17 mmol, 60% in mineral oil) was added to a mixture of 5-((4-chlorophenyl)amino)picolinaldehyde (3.70 g, 15.9 mmol), (bromomethyl)cyclopropane (6.44 g, 47.7 mmol) and DMF (70 mL) at 0° C. The mixture was stirred at rt for 5 h. Water was added and the mixture was stirred for another 0.5 h. Extractive workup (EtOAc, water, brine), drying (Na₂SO₄) and purification by chromatography gave the sub-title compound. Yield: 3.60 g (79%).

### (c) 5-(5-((4-Chlorophenyl)(cyclopropylmethyl)amino)picolinoyl)-2-(2,3-dihydro-benzo[b][1,4]dioxine-6-carbonyl)benzoic acid

The title compound was prepared from 5-((4-chlorophenyl)(cyclo-propylmethyl)amino)picolinaldehyde and 3,4-ethylenedioxybenzoyl chloride in accordance with Example 2, steps (b-f). ¹H NMR (DMSO-d₆) δ: 13.6-13.0 (1H, br s) 8.56-8.48 (1H, m) 8.18 (1H, d, J=2.7 Hz) 8.17-8.08 (1H, m) 8.03 (1H, d, J=9.0 Hz) 8.63-7.52 (2H, m) 7.46-7.33 (3H, m) 7.29 (1H, dd, J=9.0, 2.7 Hz) 7.18-7.04 (2H, m) 6.98-6.91 (1H, m) 4.35-4.30 (2H, m) 4.30-4.25 (2H, m) 3.75 (2H, d, J=6.7 Hz) 1.15-1.11 (1H, m) 0.51-0.43 (2H, m) 0.22-0.14 (2H, m). IC₅₀=21 nM.

### Example 5

### 2-Benzoyl-5-(5-(4-chlorobenzoyl)picolinoyl)benzoic acid

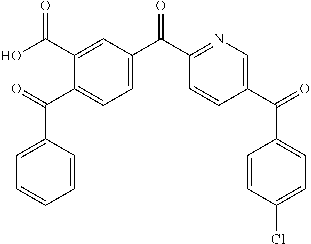 [SMILES: O=C(c1ccc(Cl)cc1)c1ccc(C(=O)c2ccc(C(=O)c3ccccc3)c(C(=O)O)c2)nc1]

### (a) 2-Benzoyl-5-bromobenzoic acid methyl ester

The sub-title compound was obtained from 5-bromo-2-iodobenzoic acid methyl ester and benzoyl chloride in accordance with Example 2, step (b).

### (b) 2-Benzoyl-5-iodobenzoic acid methyl ester

A mixture of 2-benzoyl-5-bromobenzoic acid methyl ester (5.207 g, 16.31 mmol), CuI (0.311 g, 1.63 mmol), NaI (4.889 g, 32.62 mmol), N¹,N²-dimethylethane-1,2-diamine (0.351 μL, 3.26 mmol) and dioxane (20 mL) was heated at 120° C. for 18 h in a closed vessel. Extractive workup (EtOAc, NH₄Cl (aq, sat), H₂O, brine), drying (Na₂SO₄), concentration and purification by chromatography gave the sub-title compound. Yield: 4.633 g (78%).

### (c) 2-Benzoyl-5-((5-bromo-2-pyridyl)hydroxymethyl)benzoic acid methyl ester

The sub-title compound was obtained from 2-benzoyl-5-iodobenzoic acid methyl ester and 5-bromopicolinaldehyde in accordance with Example 2, step (b).

### (d) 2-Benzoyl-5-(5-bromopicolinoyl)benzoic acid methyl ester

Pyridinium chlorochromate (0.457 g, 2.12 mmol) was added to 2-benzoyl-5-((5-bromo-2-pyridyl)hydroxymethyl)benzoic acid methyl ester (0.904 g, 2.12 mmol) in DCM (10 mL) at 0° C. After 1 h stirring at 0° C., a second portion of pyridinium chlorochromate (46 mg, 0.21 mmol) was added. After 25 min, a third portion of pyridinium chlorochromate (38 mg, 0.18 mmol) was added. After a further 1.5 h stirring at 0° C. the mixture was diluted with EtOAc. Extractive workup (EtOAc, HCl (aq, 1M), brine), drying (Na₂SO₄), concentration and purification by chromatography gave the sub-title compound. Yield: 373 mg (41%).

### (e) 2-Benzoyl-5-(5-(4-chlorobenzoyl)picolinoyl)benzoic acid

The title compound was prepared from 2-benzoyl-5-(5-bromopicolinoyl)benzoic acid methyl ester and 4-chlorobenzoyl chloride in accordance with Example 2, steps (d-f).

¹H NMR (DMSO-d₆) δ: 13.5-13.4 (1H, br s) 9.05-9.03 (1H, m) 8.62-8.59 (1H, m) 8.40 (1H, dd, J=8.1, 2.2 Hz) 8.35 (1H, dd, J=7.9, 1.7 Hz) 8.27-8.24 (1H, m) 7.91-7.86 (2H, m) 7.71-7.61 (6H, m) 7.56-7.50 (2H, m). IC₅₀=1286 nM.

### Example 6

### 5-(5-(4-Chlorobenzyl)picolinoyl)-2-(3-methoxybenzoyl)benzoic acid

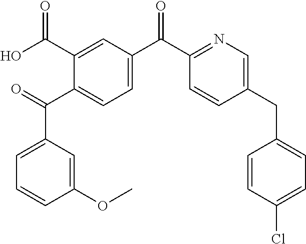 [SMILES: COc1cccc(C(=O)c2ccc(C(=O)c3ccc(Cc4ccc(Cl)cc4)cn3)cc2C(=O)O)c1]

### (a) 5-(5-Bromopicolinoyl)-2-(3-methoxybenzoyl)benzoic acid methyl ester

The sub-title compound was obtained from 5-bromo-2-iodobenzoic acid methyl ester and m-anisoyl chloride in accordance with Example 5, steps (a-d).

### (b) 5-(5-(4-Chlorobenzyl)picolinoyl)-2-(3-methoxybenzoyl)benzoic acid methyl ester

A mixture of potassium(4-chlorobenzyl)trifluoroborate (128 mg, 0.55 mmol), 5-(5-bromopicolinoyl)-2-(3-methoxybenzoyl)benzoic acid methyl ester (250 mg, 0.55 mmol), PdCl₂(dppf) CH₂Cl₂ (40 mg, 0.05 mmol), Cs₂CO₃ (538 mg, 1.65 mmol), water (0.3 mL) and THF (4 mL) was heated at rx for 19 h. Concentration and extractive workup (EtOAc, HCl (aq, 1M), brine), drying (Na₂SO₄) and purification by chromatography gave the sub-title compound. Yield: 125 mg (45%).

### (c) 5-(5-(4-Chlorobenzyl)picolinoyl)-2-(3-methoxybenzoyl)benzoic acid

The title compound was obtained after hydrolysis of 5-(5-(4-chlorobenzyl)picolinoyl)-2-(3-methoxybenzoyl)benzoic acid methyl ester in accordance with Example 2, step (f). ¹H NMR (DMSO-d₆) δ: 13.7-13.2 (1H, br s) 8.71 (1H, d, J=2.0 Hz) 8.56 (1H, d, J=1.5 Hz) 8.29 (1H, dd, J=8.0, 1.5 Hz) 8.06 (1H, d, J=8.0 Hz) 7.93 (1H, dd, J=8.0, 2.0 Hz) 7.57 (1H, d, J=8.0 Hz) 7.45-7.33 (5H, m) 7.27-7.19 (2H, m) 7.10 (1H, d, J=8.0 Hz) 4.13 (2H, s) 3.80 (3H, s). IC₅₀=155 nM.

### Example 7

### 5-(5-((4-Chlorophenyl)(methyl)amino)picolinoyl)-2-(3-hydroxybenzoyl)benzoic acid

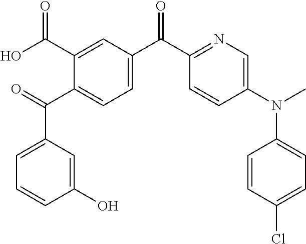 [SMILES: CN(c1ccc(Cl)cc1)c1ccc(C(=O)c2ccc(C(=O)c3cccc(O)c3)c(C(=O)O)c2)nc1]

### (a) 5-(5-((4-Chlorophenyl)(methyl)amino)picolinoyl)-2-(3-hydroxybenzoyl)-benzoic acid methyl ester

BBr₃ (156 mg, 0.621 mmol) was added dropwise to a solution of 5-(5-((4-chlorophenyl)(methyl)amino)picolinoyl)-2-(3-methoxybenzoyl)benzoic acid methyl ester (prepared in accordance to Example 2 step (e) from 5-(5-((4-chlorophenyl)(methyl)amino)picolinoyl)-2-trimethylstannanylbenzoic acid methyl ester and m-anisoyl chloride, 80 mg, 0.155 mmol) in DCM (3 mL) at −10° C. The mixture was stirred at −10° C. for 1 h and at rt for 30 min. An additional amount of BBr₃ (156 mg, 0.621 mmol) was added at −10° C. and stirring was continued for 3 h at −10° C. MeOH was added and the mixture was stirred at rt for 30 min. Concentration, extractive workup (EtOAc, H₂O, brine), drying (Na₂SO₄) and purification by chromatography gave the sub-title compound. Yield: 30 mg (38%).

### (b) 5-(5-((4-Chlorophenyl)(methyl)amino)picolinoyl)-2-(3-hydroxybenzoyl)-benzoic acid

The title compound was obtained from 5-(5-((4-chlorophenyl)-(methyl)amino)picolinoyl)-2-(3-hydroxybenzoyl)benzoic acid methyl ester in accordance with Example 2, step (f). ¹H NMR (DMSO-d₆) δ: 13.6-13.2 (1H, br s) 9.76 (1H, s) 8.52 (1H, d, J=1.4 Hz) 8.30-8.17 (2H, m) 8.02 (1H, d, J=9.0 Hz) 7.59-7.43 (3H, m) 7.42-7.21 (4H, m) 7.11-6.93 (3H, m) 3.40 (3H, s). IC₅₀=83 nM.

### Examples 9-12, 15-21

The title compounds were prepared from 5-{5-[(4-chlorophenyl)(methyl)amino]-picolinoyl}-2-trimethylstannanylbenzoic acid methyl ester and the appropriate acid chloride in accordance with Example 2, steps (e) and (f), see Table 2.

### Examples 8, 22-24

The title compounds were prepared from 5-((4-chlorophenyl)-(cyclopropylmethyl)amino)picolinaldehyde (Example 3, step (b) and the appropriate acid chloride in accordance with Example 2 steps (b-f) see Table 1.

### Example 13

The title compound was prepared from 5-(5-bromopicolinoyl)-2-(3-methoxybenzoyl)benzoic acid methyl ester (prepared in accordance with Example 5, steps (a-d) using m-anisoyl chloride in step (a)), followed by amination with 4-choroaniline, alkylation with (bromomethyl)cyclopropane in accordance with Example 4 steps (a-b) and hydrolysis in accordance with Example 2 step (f).

### Example 14

The title compound was prepared in accordance with Example 7, steps (a-b) using the penultimate intermediate in Example 13.

TABLE 2

| | (in which the compounds may exist in cyclised form; see above) | |
|---|---|---|
| | | IC₅₀ |
| | Chemical structure | (nM) |
| Ex- | Name | |
| ample | ¹H NMR (DMSO-d₆, δ) | |
| 8 | 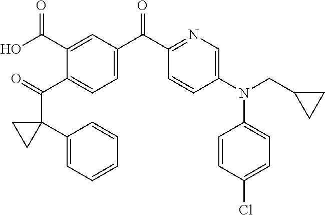 [SMILES: O=C(c1ccc(C(=O)C2(c3ccccc3)CC2)c(C(=O)O)c1)c1ccc(N(CC2CC2)c2ccc(Cl)cc2)cn1] | 41 |
| | 5-(5-((4-Chlorophenyl)(cyclopropylmethyl)amino)picolinoyl)- | |
| | 2-(1-phenylcyclopropanecarbonyl)benzoic acid | |
| | 8.34-8.24 (1H, m) 8.11 (1H, d, J = 2.7 Hz) 8.08-7.97 (1H, m) 7.93 | |
| | (1H, d, J = 9.0 Hz) 7.63-7.48 (2H, m) 7.44-7.34 (4H, m) 7.31-7.25 | |
| | (1H, m) 7.23 (1H, dd, J = 9.0, 2.7 Hz) 7.17-7.06 (3H, m) 3.70 | |
| | (2H, d, J = 6.7 Hz) 1.64-1.53 (2H, m) 1.34-122 (2H, m) 1.12-1.07 | |
| | (1H, m) 0.49-0.39 (2H, m) 0.20-0.10 (2H, m) | |
| 9 | 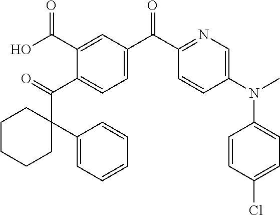 [SMILES: CN(c1ccc(Cl)cc1)c1ccc(C(=O)c2ccc(C(=O)C3(c4ccccc4)CCCCC3)c(C(=O)O)c2)nc1] | 32 |
| | 5-(5-((4-Chlorophenyl)(methyl)amino)picolinoyl)-2- | |
| | (1-phenylcyclohexanecarbonyl)benzoic acid | |
| | 13.7-13.3 (1H, br s) 8.43-7.94 (2H, m) 8.18 (1H, d, J = 2.7 Hz) | |
| | 7.98 (1H, d, J = 9.0 Hz) 7.56-7.51 (2H, m) 7.47-7.12 (6H, m) | |
| | 7.40-7.36 (2H, m) 7.28 (1H, dd, J = 9.0, 2.7 Hz) 3.40 (3H, s) | |
| | 2.41-2.29 (1H, m) 2.12-1.86 (2H, m) 1.60-1.53 (2H, m) 1.51- | |
| | 1.45 (1H, m) 1.32-1.01 (4H, m) | |
| 10 | 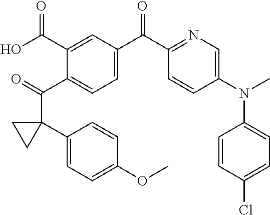 [SMILES: COc1ccc(C2(C(=O)c3ccc(C(=O)c4ccc(N(C)c5ccc(Cl)cc5)cn4)cc3C(=O)O)CC2)cc1] | 22 |
| | 5-(5-((4-Chlorophenyl)(methyl)amino)picolinoyl)-2- | |
| | (1-(4-methoxyphenyl)cyclopropanecarbonyl)benzoic acid | |
| | 13.6-13.3 (1H, br s) 8.37-8.26 (1H, m) 8.18 (1H, d, J = 2.7 Hz) | |
| | 8.16-8.05 (1H, m) 7.96 (1H, d, J = 9.0 Hz) 7.57-7.50 (2H, m) | |
| | 7.48-7.33 (3H, m) 7.28 (1H, dd, J = 9.0, 2.7 Hz) 7.27-7.10 (2H, | |
| | m) 6.78-6.59 (2H, m) 3.63 (3H, s) 3.40 (3H, s) 1.71-1.48 | |
| | (2H, m) 1.46-1.16 (2H, m) | |
| 11 | 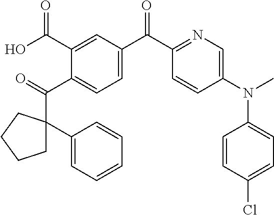 [SMILES: CN(c1ccc(Cl)cc1)c1ccc(C(=O)c2ccc(C(=O)C3(c4ccccc4)CCCC3)c(C(=O)O)c2)nc1] | 76 |
| | 5-(5-((4-Chlorophenyl)(methyl)amino)picolinoyl)-2- | |
| | (1-phenylcyclopentanecarbonyl)benzoic acid | |
| | 8.27-8.00 (2H, m) 8.18 (1H, d, J = 2.7 Hz) 7.97 (1H, d, J = 9.0 | |
| | Hz) 7.57-7.50 (2H, m) 7.42-7.10 (6H, m) 7.39-7.36 (2H, m) | |
| | 7.28 (1H, dd, J = 9.0, 2.7 Hz) 3.40 (3H, s) 2.46-2.33 (2H, m) | |
| | 2.32-2.15 (2H, m) 1.78-1.70 (2H, m) 1.53-1.36 (2H, m) 38519 | |
| 12 | 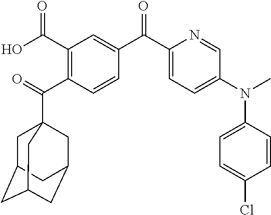 [SMILES: CN(c1ccc(Cl)cc1)c1ccc(C(=O)c2ccc(C(=O)C34CC5CC(CC(C5)C3)C4)c(C(=O)O)c2)nc1] | 65 |
| | 2-(Adamantane-1-carbonyl)-5-(5-((4-chlorophenyl) | |
| | (methyl)amino)picolinoyl)benzoic acid | |
| | 8.47-8.32 (1H, m) 8.31-8.24 (1H, m) 8.22 (1H, d, J = 2.7 Hz) | |
| | 8.04 (1H, d, J = 9.0 Hz) 7.92-7.74 (1H, m) 7.59-7.48 (2H, m) | |
| | 7.42-7.37 (2H, m) 7.31 (1H, dd, J = 9.0, 2.7 Hz) 3.42 (3H, s) | |
| | 1.99-1.94 (3H, m) 1.89-1.52 (12H, m) | |
| 13 | 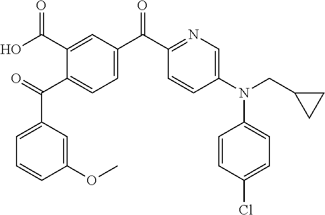 [SMILES: COc1cccc(C(=O)c2ccc(C(=O)c3ccc(N(CC4CC4)c4ccc(Cl)cc4)cn3)cc2C(=O)O)c1] | 18 |
| | 5-(5-((4-Chlorophenyl)(cyclopropylmethyl)amino)picolinoyl)- | |
| | 2-(3-methoxybenzoyl)benzoic acid | |
| | 13.6-13.2 (1H, br s) 8.54 (1H, d, J = 1.5 Hz) 8.28 (1H, dd, J = | |
| | 7.9, 1.5 Hz) 8.17 (1H, d, J = 2.8 Hz) 8.04 (1H, d, J = 8.9 Hz) | |
| | 7.62-7.49 (3H, m) 7.47-7.36 (3H, m) 7.34-7.16 (3H, m) 7.10 | |
| | (1H, d, J = 7.6 Hz) 3.80 (3H, s) 3.73 (2H, d, J = 6.6 Hz) 1.17-1.02 | |
| | (1H, m) 0.52-0.38 (2H, m) 0.22-0.10 (2H, m) | |
| 14 | 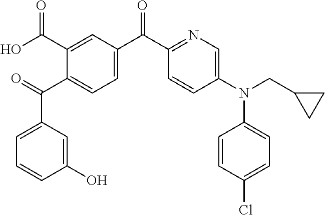 [SMILES: O=C(c1ccc(C(=O)c2cccc(O)c2)c(C(=O)O)c1)c1ccc(N(CC2CC2)c2ccc(Cl)cc2)cn1] | 30 |
| | 5-(5-((4-Chlorophenyl)(cyclopropylmethyl)amino)picolinoyl)- | |
| | 2-(3-hydroxybenzoyl)benzoic acid | |
| | 13.6-13.2 (1H, br s) 9.77 (1H, s) 8.53 (1H, d, J = 1.4 Hz) 8.25 | |
| | (1H, dd, J = 7.9, 1.4 Hz) 8.15 (1H, d, J = 2.8 Hz) 8.02 (1H, d, | |
| | J = 8.9 Hz) 7.60-7.45 (3H, m) 7.44-7.34 (2H, m) 7.34-7.20 | |
| | (2H, m) 7.10-6.94 (3H, m) 3.72 (2H, d, J = 6.6 Hz) 1.14-0.99 | |
| | (1H, m) 0.50-0.36 (2H, m) 0.20-0.08 (2H, m) | |
| 15 | 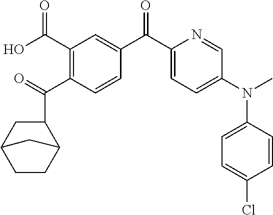 [SMILES: CN(c1ccc(Cl)cc1)c1ccc(C(=O)c2ccc(C(=O)C3CC4CCC3C4)c(C(=O)O)c2)nc1] | 94 |
| | 2-(Bicyclo[2.2.1]heptane-2-carbonyl)-5-(5-((4-chlorophenyl) | |
| | (methyl)amino)-picolinoyl)benzoic acid | |
| | 13.8-13.2 (1H, br s) 8.42-8.33 (1H, m) 8.30-8.12 (1H, m) 8.21 | |
| | (1H, d, J = 2.7 Hz) 8.03 (1H, d, J = 9.0 Hz) 7.76-7.45 (3H, m) | |
| | 7.43-7.36 (2H, m) 7.30 (1H, dd, J = 9.0, 2.7 Hz) 3.41 (3H, s) | |
| | 2.46-2.30 (1H, m) 2.27-2.22 (1H, m) 1.64-0.91 (9H, m) | |
| 16 | 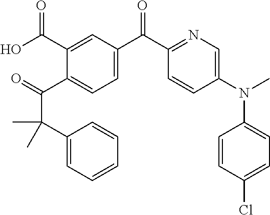 [SMILES: CN(c1ccc(Cl)cc1)c1ccc(C(=O)c2ccc(C(=O)C(C)(C)c3ccccc3)c(C(=O)O)c2)nc1] | 55 |
| | 5-(5-((4-Chlorophenyl)(methyl)amino)picolinoyl)-2- | |
| | (2-methyl-2-phenylpropanoyl)benzoic acid | |
| | 8.45-8.09 (1H, m) 8.19 (1H, d, J = 2.7 Hz) 8.20-7.93 (1H, m) | |
| | 7.99 (1H, d, J = 9.0 Hz) 7.58-7.50 (2H, m) 7.49-7.35 (4H, m) | |
| | 7.35-6.92 (4H, m) 7.28 (1H, dd, J = 9.0, 2.7 Hz) 3.40 (3H, s) | |
| | 1.63-1.39 (6H, m) | |
| 17 | 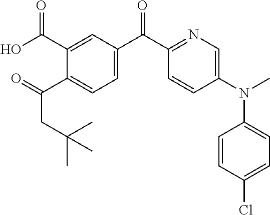 [SMILES: CN(c1ccc(Cl)cc1)c1ccc(C(=O)c2ccc(C(=O)CC(C)(C)C)c(C(=O)O)c2)nc1] | 235 |
| | 5-(5-((4-Chlorophenyl)(methyl)amino)picolinoyl)-2- | |
| | (3,3-dimethylbutanoyl)benzoic acid | |
| | 8.41-8.32 (1H, m) 8.20 (1H, d, J = 2.7 Hz) 8.13-8.04 (1H, m) | |
| | 7.99 (1H, d, J = 9.0 Hz) 7.57-7.49 (2H, m) 7.46-7.35 (3H, m) | |
| | 7.30 (1H, dd, J = 9.0, 2.7 Hz) 3.41 (3H, s) 2.78-2.69 (2H, m) | |
| | 1.00 (9H, s) | |
| 18 | 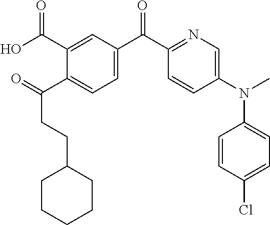 [SMILES: CN(c1ccc(Cl)cc1)c1ccc(C(=O)c2ccc(C(=O)CCC3CCCCC3)c(C(=O)O)c2)nc1] | 59 |
| | 5-(5-((4-Chlorophenyl)(methyl)amino)picolinoyl)-2- | |
| | (3-cyclohexylpropanoyl)benzoic acid | |
| | 8.42-8.31 (1H, m) 8.30-8.19 (1H, m) 8.21 (1H, d, J = 2.7 Hz) | |
| | 8.03 (1H, d, J = 9.0 Hz) 7.74-7.58 (1H, m) 7.57-7.50 (2H, m) | |
| | 7.42-7.36 (2H, m) 7.31 (1H, dd, J = 9.0, 2.7 Hz) 3.41 (3H, s) | |
| | 2.47-2.24 (1H, m) 1.93-1.50 (6H, m) 1.35-1.05 (6H, m) 0.89- | |
| | 0.79 (2H, m) | |
| 19 | 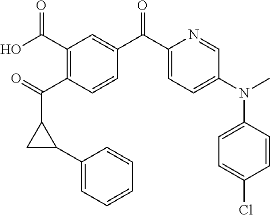 [SMILES: CN(c1ccc(Cl)cc1)c1ccc(C(=O)c2ccc(C(=O)C3CC3c3ccccc3)c(C(=O)O)c2)nc1] | 141 |
| | 5-(5-((4-Chlorophenyl)(methyl)amino)picolinoyl)-2- | |
| | (2-phenylcyclopropanecarbonyl)benzoic acid | |
| | 13.8-13.0 (1H, br s) 8.45-8.35 (1H, m) 8.19 (1H, d, J = 2.7 Hz) | |
| | 8.19-8.11 (1H, m) 8.00 (1H, d, J = 9.0 Hz) 7.59-7.45 (3H, m) | |
| | 7.41-7.36 (2H, m) 7.31-7.18 (6H, m) 3.40 (3H, s) 2.65-2.52 | |
| | (2H, m) 1.73-1.66 (1H, m) 1.57-1.48 (1H, m) | |
| 20 | 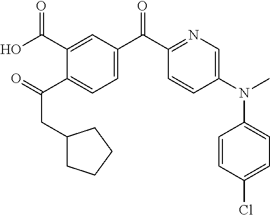 [SMILES: CN(c1ccc(Cl)cc1)c1ccc(C(=O)c2ccc(C(=O)CC3CCCC3)c(C(=O)O)c2)nc1] | 81 |
| | 5-(5-((4-Chlorophenyl)(methyl)amino)picolinoyl)-2- | |
| | (2-cyclopentylacetyl)benzoic acid | |
| | 8.39-8.32 (1H, m) 8.26-8.13 (2H, m) 8.00 (1H, d, J = 9.0 Hz) | |
| | 7.68-7.45 (3H, m) 7.42-7.32 (2H, m) 7.28 (1H, dd, J = 9.0, | |
| | 2.8 Hz) 3.39 (3H, s) 2.69-2.45 (1H, m) 2.10-1.84 (1H, m) | |
| | 1.79-1.30 (6H, m) 1.26-0.95 (3H, m) | |
| 21 | 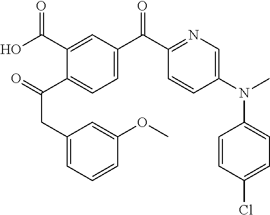 [SMILES: COc1cccc(CC(=O)c2ccc(C(=O)c3ccc(N(C)c4ccc(Cl)cc4)cn3)cc2C(=O)O)c1] | 192 |
| | 5-(5-((4-Chlorophenyl)(methyl)amino)picolinoyl)-2-(2-3- | |
| | methoxyphenyl)acetyl)benzoic acid | |
| | 8.41-8.08 (2H, m) 8.20 (1H, d, J = 2.7 Hz) 8.00 (1H, d, J = | |
| | 9.0 Hz) 7.79-7.44 (3H, m) 7.43-7.35 (2H, m) 7.29 (1H, dd, J = | |
| | 9.0, 2.7 Hz) 7.19-7.04 (1H, m) 6.90-6.54 (3H, m) 3.80-3.33 | |
| | (2H, m) 3.64 (3H, s) 3.41 (3H, s) | |
| 22 | 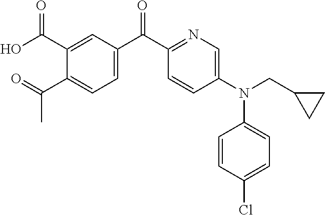 [SMILES: CC(=O)c1ccc(C(=O)c2ccc(N(CC3CC3)c3ccc(Cl)cc3)cn2)cc1C(=O)O] | 307 (Test 2) |
| | 2-Acetyl-5-(5-((4-chlorophenyl)(cyclopropylmethyl) | |
| | amino)picolinoyl)benzoic acid | |
| | 8.36-8.31 (1H, m) 8.25 (1H, d, J = 7.0 Hz) 8.15 (1H, d, J = | |
| | 2.7 Hz) 8.01 (1H, d, J = 9.0 Hz) 7.82-7.62 (1H, m) 7.61-7.49 | |
| | (2H, m) 7.43-7.34 (2H, m) 7.26 (1H, dd, J = 9.0, 2.7 Hz) 3.72 | |
| | (2H, d, J = 6.7 Hz) 2.3-1.6 (3H, br s) 1.16-1.04 (1H, m) 0.53- | |
| | 0.36 (2H, m) 0.23-0.08 (2H, m) | |
| 23 | 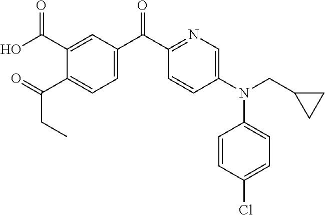 [SMILES: CCC(=O)c1ccc(C(=O)c2ccc(N(CC3CC3)c3ccc(Cl)cc3)cn2)cc1C(=O)O] | 129 (Test 2) |
| | 5-(5-((4-Chlorophenyl)(cyclopropylmethyl)amino) | |
| | picolinoyl)-2-propionylbenzoic acid | |
| | 8.42-8.32 (1H, m) 8.32-8.18 (1H, m) 8.15 (1H, d, J = 2.7 Hz) | |
| | 8.02 (1H, d, J = 9.0 Hz) 7.84-7.60 (1H, m) 7.59-7.51 (2H, m) | |
| | 7.45-7.34 (2H, m) 7.26 (1H, dd, J = 9.0, 2.7 Hz) 3.73 (2H, d, J = | |
| | 6.7 Hz) 2.41-1.93 (2H, m) 1.16-1.05 (1H, m) 1.07-0.74 (3H, m) | |
| | 0.50-0.38 (2H, m) 0.21-0.09 (2H, m) | |
| 24 | 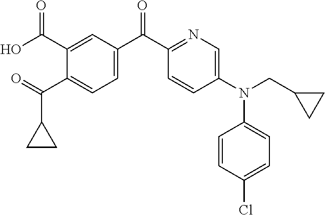 [SMILES: O=C(c1ccc(C(=O)C2CC2)c(C(=O)O)c1)c1ccc(N(CC2CC2)c2ccc(Cl)cc2)cn1] | 424 (Test 2) |

5-(5-((4-Chlorophenyl)(cyclopropylmethyl)amino)
picolinoyl)-2-(cyclopropanecarbonyl)benzoic acid
8.41-8.33 (1H, m) 8.13 (1H, d, J = 2.7 Hz) 8.05 (1H, dd, J = 7.8,
1.2 Hz) 7.98 (1H, d, J = 9.0 Hz) 7.62-7.51 (2H, m) 7.43-7.32 (3H,
m) 7.26 (1H, dd, J = 9.0, 2.7 Hz) 3.71 (2H, d, J = 6.7 Hz) 2.30-2.21
(1H, m) 1.13-1.06 (1H, m) 1.02-0.91 (4H, m) 0.48-0.42 (2H, m)
0.18-0.13 (2H, m)

### Example 25

By definition, the solubility of a compound is the maximum quantity of compound that can dissolve in a certain quantity of solvent at a specified temperature.

The method described here was developed to accurately determine the aqueous solubility of compounds of the invention in buffer solution at a given pH. The test is built as a classical thermodynamic solubility method with an assumption that saturation of solution incubated with an excess of solid material, is achieved after 24 h.

Solid material (1 mg) of test compound is added to a glass vial followed by 1 ml of buffer solution (pH 7.4 if another pH not is stated). The solution is left on an orbital shaker for 24 h at 20° C. After incubation, the remaining solid material is separated from solution and the solubility is quantified using LC-MS/MS.

### Method and Materials

### Preparation of USP Phosphate Buffer, pH 7.4

Prepare 0.2 M monobasic potassium phosphate solution by dissolving 27.22 g of KH₂PO₄ (MW=136.09 g/mol) in water, dilute with water to 1000 mL.

Add 250 mL of the monobasic potassium phosphate solution in a 1000 mL volumetric flask together with 195.5 mL 0.2M NaOH (aq). Add water to 1000 mL. Check pH.

### Test Compounds

Compounds of the invention/examples to be tested were provided as solid material, weighed into glass vials (2 mL). Each vial contains approximately 1 mg solid compound, two vials/compound are prepared, i.e. duplicate samples of each compound. If a freshly prepared 10 mM DMSO stock solution was available, this solution was used for MS optimization and preparation of standards. If a DMSO-stock solution was not available, a 10 mM solution from solid material 1 mg was prepared.

### Methods

### Day one

Test compounds are employed as solid material. Since duplicate samples are prepared for each compound, each test compound arrives in two vials (1+1 mg). Samples are denoted as sample 1 and 2.
Note amount of substance in each vial. Add USP phosphate buffer pH 7.4 (1 mL) to each sample vial and seal vials using screw caps. Determine whether the solutions are saturated or if compounds are dissolved, note appearance and time. Start incubation; the samples are incubated on an orbital shaker (450 rpm) at 20° C. for 24 hours.

### Day Two

Take out the 1 mM DMSO-stock solutions of compounds that are to be tested from freezer. If no 1 mM solutions are available, use master solutions with concentration of 10 mM. Allow the solutions to thaw at rt.
Collect two 96-deep well plates, glass inserts, sealing film, and an 8-channel expandable pipette with tall tips.
Abort shaking after 24 hour.
Note if samples are saturated or if compounds have dissolved.
Transfer 720 μL of sample slurry from each sample vial into glass inserts in a 96-deep well plate using an 8-channel expandable pipette. Samples 1 and 2 are transferred to separate plates. Thus, all samples 1 are placed in one plate and all samples 2 in the second plate.
Seal the plates using sealing film.
Centrifuge the plates at 3000 rpm for 15 min at rt. Set acceleration to 9 and brake to 7 (9=fastest acc/brake, 0=slowest acc/brake).
Transfer the supernatants to new glass inserts using an 8-channel pipette, centrifuge samples a second time.
Repeat the procedure above, i.e. centrifuge samples a third time.
After the third centrifugation, transfer supernatant to glass vials, seal vials with screw caps. Control that the solutions are clear and that no particles are present in sample. If solid material is observed, an additional centrifugation step is performed.
Standards are prepared from DMSO-stock solutions (1 or 10 mM). For each compound, transfer 10 of 1 mM solution to a glass vial, add DMSO (90 μL) to reach a final concentration of 100 μM, seal with screw cap and mix solution quickly on a vortex mixer. Further dilute the 100 μM solution with a 1:1 mixture of acetonitrile and USP phosphate buffer (pH 7.4) to standards with concentration 1000, 500, 100, 25, 2, and 1 nM. Standards are prepared in test tubes.

Preparation of 1000 nM standard: Transfer 10 μL from 100 μM DMSO-solution to a test tube, add 990 μL of acteonitrile:buffer, mix solution quickly on a vortex mixer

Preparation of 500 nM standard: Transfer 500 μL from 1000 nM standards, dilute with 500 μL acteonitrile:buffer, mix solution quickly on a vortex mixer.

Preparation of 100 nM standard: Transfer 200 μL from 500 nM standards, dilute with 800 μL acteonitrile:buffer, mix solution quickly on a vortex mixer.

Preparation of 25 nM standard: Transfer 250 μL from 100 nM standards, dilute with 750 μL acteonitrile:buffer, mix solution quickly on a vortex mixer.

Preparation of 2 nM standard: Transfer 80 μL from 25 nM standards, dilute with 920 μL acteonitrile:buffer, mix solution quickly on a vortex mixer.

Preparation of 1 nM standard: Transfer 40 μL from 25 nM standards, dilute with 960 μL acteonitrile:buffer, mix solution quickly on a vortex mixer. Preparation of 0 nM standard: use acetonitrile:buffer
Standards are transferred to a deep well plate containing glass inserts (700 μL) starting with compound 1 in row A, compound 2 in row B etc. Place standards as indicated in scheme 1. Seal the plate with a cap mat.

Scheme 1. Standard solutions
(nM) are place as indicated.

| | 1 | 2 | 3 | 4 | 5 | 6 | 7 | 8 | 9 | 10 | 11 |
|---|---|---|---|---|---|---|---|---|---|---|---|
| A | 0 | 1 | 2 | 25 | 100 | 500 | 1000 | | | | |
| B | 0 | 1 | 2 | 25 | 100 | 500 | 1000 | | | | |
| C | 0 | 1 | 2 | 25 | 100 | 500 | 1000 | | | | |
| D | 0 | 1 | 2 | 25 | 100 | 500 | 1000 | | | | |
| E | 0 | 1 | 2 | 25 | 100 | 500 | 1000 | | | | |
| F | | | | | | | | | | | |
| G | | | | | | | | | | | |
| H | | | | | | | | | | | |

Place standards in autosampler.
Try out the dilution of solubility samples so that their response will be within the standard curve. Dilute solubility sample one (or two) with the 1:1 mixture of acetonitrile:buffer. For highly soluble compounds, start with a 5000× dilution, for less soluble compounds start with a 1000× dilution.
Control the dilutions: Analyze standards (1000 nM) and the test dilutions using sample list format templat in MassLynx. In Microsoft explorer, create/load the MS-files.
Use the dilution that gives rise to an acceptable response of each compound. Dilute all solubility samples twice to minimize the risk of overestimation of the solubility due to particles (undissolved material) left after centrifugation, that are dissolved in acetonitrile:buffer (i.e. all test samples are diluted twice with MeCN/buffer solution).
Place solubility samples in vials according to scheme 2. Seal the plate with a silicone/Teflon cap map.

Scheme 2. Solubility samples are placed
as indicated, starting with compound 1
in row A, compound 2 in row B etc.

| | 1 | 2 | 3 | 4 | 5 | 6 | 7 | 8 | 9 | 10 |
|---|---|---|---|---|---|---|---|---|---|---|
| A | sol1,dil1 | sol1,dil2 | sol2,dil1 | sol1,dil2 | | | | | | |
| B | sol1,dil1 | sol1,dil2 | sol2,dil1 | sol1,dil2 | | | | | | |
| C | sol1,dil1 | sol1,dil2 | sol2,dil1 | sol1,dil2 | | | | | | |
| D | sol1,dil1 | sol1,dil2 | sol2,dil1 | sol2,dil2 | | | | | | |
| E | sol1,dil1 | sol1,dil2 | sol2,dil1 | sol1,dil2 | | | | | | |
| F | | | | | | | | | | |
| G | | | | | | | | | | |
| H | | | | | | | | | | |

Place the plate in autosampler.
Make a sample list using a sample list format.
Start the LC-MS/MS analysis.

### Results and Calculations

On MS-computer, analyze the LC-MS/MS data using QuanLynx and plot the solubility data.

The thermodynamic aqueous solubilities of representative examples are presented in the table below:

| | Thermodynamic aq solubility |
|---|---|
| Example | (μM) |
| 1:1 | 2577 |
| 1:4 | 2209 |
| 1:5 | 2477 |
| 1:17 | 153 |
| 1:19 | 680 |
| 8 | 125 |
| 9 | 7 |
| 10 | 403 |
| 12 | 15 |
| 14 | 1209 |
| 6 | 1902 |
| 15 | 1982 |
| 18 | 37 |
| 22 | 1859 |

1. A compound of formula I, 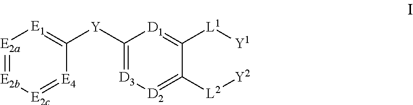
wherein
one of L² and L³ is —C(O)-A¹⁷- and the other is:
a single bond, —S(O)ₙ₁—, —C(R^{y4})(R^{y5})-A¹⁶, —N(R^{17a})-A¹⁶-, —OA¹⁷- or —C(O)-A¹⁷-;
one of E₂ₐ, E_{2b} and E_{2c} is —C(-L³-Y³)═ and the other two are respectively E₂ and E₃;
Y is —C(O)— or —C(═N—OR²⁸)—;
R²⁸ is hydrogen or C₁₋₆ alkyl optionally substituted by one or more fluoro atoms;
one or two of D₁, D₂ and D₃ are —N═; and/or
one or two of E₁, E₂, E₃ and E₄ are —N═; and
those (or the) remaining D₁, D₂ and D₃ group(s) are each independently —C(R¹)═; and
those remaining E₁, E₂, E₃ and E₄ groups are each independently —C(R²)═;
each R¹ is independently hydrogen or X¹;
each R² is independently hydrogen or X²;
Y¹ is —C(O)OR^{9a} or 5-tetrazolyl;
R^{9a} is:
(i) hydrogen; or
(ii) C₁₋₈ alkyl or a heterocycloalkyl group, both of which are optionally substituted by one or more substituents, wherein the substituents are independently G¹ or Z¹;
one of Y² and Y³ is an aryl group or a heteroaryl group optionally substituted by one or more, A and the other is either:
(a) an aryl group or a heteroaryl group optionally substituted by one or more A; or
(b) C₁₋₁₂ alkyl or a heterocycloalkyl group, both of which are optionally substituted by one or more G¹ or Z¹;
each A is independently:
I) an aryl group or a heteroaryl group, both of which are optionally substituted by one or more B;
II) C₁₋₈ alkyl or a heterocycloalkyl group, both of which are optionally substituted by one or more G¹ or Z¹; or
III) a G¹ group;
X¹, X², G¹ and B are independently halo, —R^{5a}, —C(O)R^{5b}, —CN, —NO₂, —C(O)N(R^{6a})R^{7a}, —N(R^{6b})R^{7b}, —N(R^{5c})C(O)R^{6c}, —N(R^{5d})C(O)OR^{6d}, —OR^{5e}, —OS(O)₂R^{5f}, —S(O)ₘR^{5g}, —OC(O)R^{5h} or —S(O)₂N(R^{6e})R^{7e};
R^{5b} to R^{5e}, R^{5g}, R^{5h}, R^{6a} to R^{6c}, R^{6e}, R^{7a}, R^{7b} and R^{7e} are independently H or R^{5a}; or
any of the pairs R^{6a} and R^{7a}, R^{6b} and R^{7b}, or R^{6e} and R^{7e} may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents, wherein the substituents are fluoro, ═O, —OR^{5e} and/or R^{5a};
R^{5f} and R^{6d} are independently R^{5a};
each R^{5a} is independently:
(i) C₁₋₆ alkyl optionally substituted by one or more substituents, wherein the substituents are fluoro, —CN, ═O, —OR^{8a}, —N(R^{8b})R^{8c}, —S(O)ₙR^{8d} or —S(O)₂N(R^{8e})R^{8f}; or
(ii) aryl or heteroaryl, both of which are optionally substituted by one or more substituents, wherein the substituents are halo, —CN, —OR^{8a}, —N(R^{8b})R^{8c}, —S(O)ₙR^{8d} or —S(O)₂N(R^{8e})R^{8f};
n is 0, 1 or 2;
each R^{8b}, R^{8d} and R^{8e} is independently H or C₁₋₆ alkyl optionally substituted by one or more substituents, wherein the substituents are fluoro, ═O, —OR^{11a} or —N(R^{12a})R^{12b};
each R^{8a}, R^{8c} and R^{8f} is independently H or C₁₋₃ alkyl optionally substituted by one or more substituents, wherein the substituents are F, ═O, —OR^{13a}, —N(R^{14a})R^{14b}, —S(O)₂CH₃, —S(O)₂CHF₂ and/or —S(O)₂CF₃; or
R^{8b} and R^{8c} and/or R^{8e} and R^{8f} may be linked together to form, along with the atom(s) to which they are attached, a 3- to 6-membered ring, optionally substituted by one or more substituents, wherein the substituents are fluoro or C₁₋₂ alkyl;
R^{11a} and R^{13a} are independently H or C₁₋₃ alkyl optionally substituted by one or more fluoro atoms;
R^{12a}, R^{12b}, R^{14a} and R^{14b} are independently H, —CH₃ or —CH₂CH₃;
each Z¹ is independently ═O or ═NOR^{16b};
R^{16b} is hydrogen or C₁₋₆ alkyl optionally substituted by one or more fluoro atoms;
L¹ is a single bond or —(CH₂)ₚ-Q-(CH₂)_{q}—;
Q is —C(R^{y1})(R^{y2})—, —C(O)—, —N(R^{y3})— or —O—;
p and q are independently 0, 1 or 2, wherein the sum of p and q does not exceed 2;
n1 is 0, 1 or 2;
A¹⁶ is a direct bond, —C(R^{y6})(R^{y7})—, —C(O)—, —C(O)N(R^{17b})—, —C(O)C(R^{y6})(R^{y7})— or —S(O)₂—;
A¹⁷ is a direct bond or —C(R^{y8})(R^{y9})—;
each R^{y1}, R^{y2}, R^{y4}, R^{y5}, R^{y6}, R^{y7}, R^{y8} and R^{y9} is independently H, fluoro or C₁₋₃ alkyl optionally substituted by one or more fluoro atoms; or
R^{y1} and R^{y2}, R^{y4} and R^{y5}, R^{y6} and R^{y7} and R^{y8} and R^{y9} may be linked together to form a 3- to 6-membered ring optionally substituted by one or more substituents, wherein the substituents are fluoro or C₁₋₂ alkyl;
R^{y3} is hydrogen or C₁₋₃ alkyl;
R^{17a} and R^{17b} are independently hydrogen, C₁₋₆ alkyl optionally substituted by one or more substituents, wherein the substituents are heterocycloalkyl, aryl, heteroaryl which latter two groups are optionally substituted by one or more substituents, wherein the substituents are R³⁰, fluoro, —CN, —OR¹⁹ or ═O, aryl or heteroaryl both of which latter two groups are optionally substituted by one or more substituents, wherein the substituents are R³¹;
R³⁰ and R³¹ are independently halo, —R^{18a}, —C(O)R^{18b}, —CN, —C(O)N(R^{18c})R^{18d}, —N(R^{18e})R^{18f}, —N(R^{18g})C(O)R^{18h}, —N(R¹⁸ⁱ)C(O)OR^{18j}, —OR^{18k}, —OS(O)₂R^{18m}, —S(O)ₘR¹⁸ⁿ, —OC(O)R^{18p} or —S(O)₂N(R^{18q})R^{18r});
m is 0, 1 or 2;
R^{18a}, R^{18b}, R^{18c}, R^{18d}, R^{18e}, R^{18f}, R^{18g}, R^{18h}, R¹⁸ⁱ, R^{18k}, R¹⁸ⁿ, R^{18p}, R^{18q} and R^{18r} are independently hydrogen or C₁₋₃ alkyl optionally substituted by one or more fluoro atoms;
R^{18j} and R^{18m} are independently C₁₋₃ alkyl optionally substituted by one or more fluoro atoms;
R¹⁹ is hydrogen or C₁₋₆ alkyl optionally substituted by one or more fluoro atoms;
or a pharmaceutically-acceptable salt thereof.

2. The compound of claim 1, further represented by a formula: 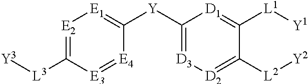
wherein E₁ is —N═;
E₄ is —N═ or —C(R²)═;
E₂ and E₃ are independently —C(R²)═;
each R² is independently hydrogen;
D₂ is —C(R¹)═;
D₁ and D₃ are independently —C(R¹)═ or —N═;
only one of the D₁ to D₃-containing ring and the E₁ to E₄-containing ring contains a nitrogen atom;
each R¹ is hydrogen;
L¹ is a single bond;
Y¹ is —C(O)OR^{9a};
one of L² and L³ is —C(O)-A¹⁷- and the other is:
a single bond, —S(O)ₙ₁—, —C(R^{y4})(R^{y5})-A¹⁶, —N(R^{17a})-A¹⁶-, —OA¹⁷- or —C(O)-A¹⁷-;
A¹⁶ is —CH₂—, a direct bond, —C(O)— or —S(O)₂—;
A¹⁷ is a direct bond or —C(R^{y8})(R^{y9})—;
R^{y8} and R^{y9} is independently H, fluoro, or C₁₋₃ alkyl optionally substituted by one or more fluoro atoms, or are linked together to form a 3- to 6-membered ring optionally substituted by one or more substituents, wherein the substituents are fluoro or C₁₋₂ alkyl;
R^{17a} is hydrogen or C₁₋₆ alkyl optionally substituted by one or more substituents, wherein the substituents are fluoro, —CN, —OR¹⁹, heterocycloalkyl or aryl;
one of Y² and Y³ is an aryl group or a heteroaryl group, both of which groups are optionally substituted by one or more substituents, wherein the substituents are A, and the other is either an aryl group or a heteroaryl group. both of which groups are optionally substituted by one or more substituents, wherein the substituents are A, or C₁₋₁₂ alkyl or a heterocycloalkyl group, both of which are optionally substituted by one or more substituents, wherein the substituents are G¹ and/or Z¹;
A is aryl or heteroaryl, both of which are optionally substituted by one or more B substituents, or A is G¹ or C₁₋₄ alkyl optionally substituted by one or more substituents, wherein the substituents are G¹;
G¹ is halo, —R^{5a}, —OR^{5e}, —S(O)ₘR^{5g}, —C(O)N(R^{6a})R^{7a} or —N(R^{6b})R^{7b};
m is 0, 1 or 2;
B is halo;
R^{5e} is hydrogen, C₁₋₄ alkyl optionally substituted by one or more halo atoms, or aryl or heteroaryl, wherein the aryl or heteroaryl are optionally substituted by one or more substituents, wherein the substituents are fluoro, chloro or —CN;
R^{5g} is C₁₋₄ alkyl;
R^{6e} and R^{7e} is independently hydrogen or C₁₋₂ alkyl; and
Z¹ is ═O.

3. The compound of claim 1, wherein:
one of L² and L³ is —C(O)-A¹⁷- and the other is a single bond,
—OA¹⁷-, —N(R^{17a})-A¹⁶, —C(O)-A¹⁷, —S— or —S(O)—;
R^{y8} and R^{y9} are hydrogen, or, are linked together to form a cyclopropyl group;
Y² is: acyclic C₁₋₆ alkyl; phenyl; 5- or 6-membered heteroaryl; 9- or 10-membered bicyclic heteroaryl group; C₃₋₈ cycloalkyl; or a 4- to 8-membered heterocycloalkyl group, all of which groups are optionally substituted by one or more substituents, wherein the substituents are independently A, G¹ Z¹;
Y³ is phenyl optionally substituted by one or more substituents selected from A; and
G¹ is halo, —CN, —NO₂, —OR^{5e}, —S(O)ₘR^{5g} or —S(O)₂N(R^{6e})R^{7e}.

4. The compound of claim 1, wherein:
n1 is 1;
one of L² and L³ is —C(O)-A¹⁷- and the other is a single bond, —S(O)—, —C(R^{y4})(R^{y5})—,
—N(R^{17a})-A¹⁶- or —OA¹⁷-;
A¹⁶ is a direct bond, —C(O)—, —C(O)N(R^{17b})—, —C(O)C(R^{y6})(R^{y7})— or —S(O)₂—;
R^{5a} is independently C₁₋₆ alkyl optionally substituted by one or more substituents, wherein the substituents are independently fluoro, —CN, ═O, —OR^{8a}, —N(R^{8b})R^{8c}, —S(O)ₙR^{8d} or —S(O)₂N(R^{8e})R^{8f};
R^{17a} and R^{17b} is independently hydrogen, C₁₋₆ alkyl optionally substituted by one or more substituents, wherein the substituents are independently fluoro, —CN, —OR¹⁹ or ═O, aryl or heteroaryl optionally substituted by one or more substituents, wherein the substituents are independently halo, —R^{18a}, —C(O)R^{18b}, —CN, —C(O)N(R^{18c})R^{18d}, —N(R^{18e})R^{18f}, —N(R^{18g})C(O)R^{18h}, —N(R¹⁸ⁱ)C(O)OR^{18j}, —OR^{18k}, —OS(O)₂R^{18m},
—S(O)ₘR¹⁸ⁿ, —OC(O)R^{18p} or —S(O)₂N(R^{18q})R^{18r});
X¹, X², G¹ and B are independently halo, —R^{5a}, —C(O)R^{5b}, —CN, —C(O)N(R^{6a})R^{7a}, —N(R^{6b})R^{7b}, —N(R^{5c})C(O)R^{6c}, —N(R^{5d})C(O)OR^{6d}, —OR^{5e}, —OS(O)₂R^{5f}, —S(O)ₘR^{5g}, —OC(O)R^{5h} or —S(O)₂N(R^{6e})R^{7e};
each R^{8a}, R^{8b}, R^{8d} and R^{8e} is independently H or C₁₋₆ alkyl optionally substituted by one or more substituents, wherein the substituents are fluoro, ═O, —OR^{11a} or —N(R^{12a})R^{12b}; and
when L² or L³ is C(R^{y4})(R^{y5})-A¹⁶ in which A¹⁶ is other than a direct, then A¹⁶ is —C(O)—.

5. The compound of claim 1, wherein:
one of L² and L³ is —C(O)-A¹⁷, and the other is a single bond, —N(R^{17a})-A¹⁶- or —OA¹⁷-;
A¹⁶ is a direct bond, —C(O)— or —S(O)₂—;
when L³ is —N(R^{17a})-A¹⁶-, then A¹⁶ is a direct bond;
A¹⁷ is a direct bond;
R^{17a} is hydrogen or C₁₋₆ alkyl optionally substituted by one or more substituents, wherein the substituents are —OCH₃, —OCH₂CH₃ and —CN;
when R^{17a} is optionally substituted C₁₋₆ alkyl, then that group is: a linear unsaturated C₁₋₆ alkyl group, a part cyclic C₁₋₆ alkyl group; or a linear saturated C₁₋₆ alkyl group, optionally substituted by —OCH₃, —OCH₂CH₃ and/or —CN;
Y² and Y³ are independently an aryl or heteroaryl group optionally substituted by one or more substitutents selected from A;
A is aryl optionally substituted by halo or G¹;
G¹ is halo, —R^{5a}, —OR^{5e} or —S(O)ₘR^{5g};
R^{5g} is R^{5a};
R^{5a} is C₁₋₆ alkyl optionally substituted by one or more fluoro atoms;
when R^{5e} is R^{5a}, then R^{5a} is C₁₋₆ alkyl; and/or
when R^{5g} is R^{5a}, then R^{5a} is unsubstituted C₁₋₄ alkyl.

6. The compound of claim 1, wherein Y² and Y³ are independently optionally substituted phenyl, naphthyl, pyrrolyl, furanyl, thienyl, imidazolyl, oxazolyl, isoxazolyl, thiazolyl, pyrazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, or benzodioxanyl.

7. The compound of claim 6, wherein the optional substituents are independently halo; cyano; C₁₋₆ alkyl optionally substituted with one or more halo groups; heterocycloalkyl optionally substituted by one or more substituents, wherein the substituents of heterocycloalkyl are independently C₁₋₃ alkyl or ═O; —OR²⁶; —C(O)R²⁶; —C(O)OR²⁶; —N(R²⁶)R²⁷; —S(O)ₘR²⁶ wherein m is 0, 1 or 2, and wherein R²⁶ and R²⁷ are independently H, C₁₋₆ alkyl optionally substituted by one or more halo groups, or aryl optionally substituted by one or more substituents, wherein the substituents are independently halo or C₁₋₃ alkyl groups optionally substituted by one or more halo atoms.

8. (canceled)

9. A pharmaceutical formulation including a compound of claim 1, or a pharmaceutically acceptable salt thereof, in admixture with a pharmaceutically acceptable adjuvant, diluent or carrier.

10. A method of treating a disease in which inhibition of the synthesis of leukotriene C₄ is desired or required, comprising administering a compound according to claim 1, or a pharmaceutically acceptable salt thereof, to a patient in need thereof.

11. (canceled)

12. The method of claim 10 wherein the disease is a respiratory disease, inflammation or has an inflammatory component.

13. The method of claim 12 wherein the disease is an allergic disorder, asthma, childhood wheezing, a chronic obstructive pulmonary disease, bronchopulmonary dysplasia, cystic fibrosis, an interstitial lung disease, an ear nose and throat disease, an eye disease, a skin diseases, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhoea, a burn-induced oxidative injury, multiple trauma, pain, toxic oil syndrome, endotoxin chock, sepsis, a bacterial infection, a fungal infection, a viral infection, sickle cell anaemia, hypereosinofilic syndrome, or a malignancy.

14. The method of claim 13, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain.

15. (canceled)

16. A combination product comprising:
(A) a compound of claim 1, or a pharmaceutically-acceptable salt thereof; and
(B) a second therapeutic agent that is useful in the treatment of a respiratory disorder or inflammation,
wherein each of components (A) and (B) is formulated in admixture with a pharmaceutically-acceptable adjuvant, diluent or carrier.

17. The combination product of claim 16 wherein the compound and the second therapeutic agent are combined in a single pharmaceutical formulation.

18. The combination product of claim 16 which comprises a kit of parts comprising components:
(a) a pharmaceutical formulation including the compound, or a pharmaceutically-acceptable salt thereof, in admixture with a pharmaceutically-acceptable adjuvant, diluent or carrier; and
(b) a pharmaceutical formulation including the second therapeutic agent that is useful in the treatment of a respiratory disorder or inflammation in admixture with a pharmaceutically-acceptable adjuvant, diluent or carrier,
which components (a) and (b) are each provided in a form that is suitable for administration in conjunction with the other.

19. A process for the preparation of a compound of claim 1, which process comprises:
(i) for compounds of formula I in which Y is —C(O)—, oxidation of a compound of formula II, 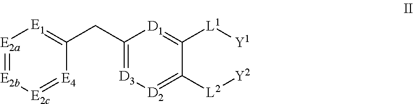
or a compound corresponding to a compound of formula II, but in which the methylene bridge is —C(H)(OH)—, wherein ring E₁, E₂ₐ, E_{2b}, E_{2c}, E_{2d}, E₄, D₁, D₂, D₃, L¹, Y¹, L² and Y² are as hereinbefore defined, in the presence of a suitable oxidising agent;
(ia) for compounds of formula I in which Y is —C(O)—, oxidation of a compound of formula IIA, 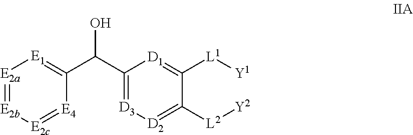
wherein ring E₁, E₂ₐ, E_{2b}, E_{2c}, E_{2d}, E₄, D₁, D₂, D₃, L¹, Y¹, L² and Y² are as defined in claim 1;
(ii) for compounds of formula I in which L² or L³ is —N(R^{17a})A¹⁶- and the other is —C(O)-A¹⁷-, reaction of a compound of formula III, 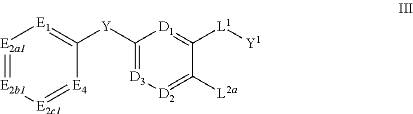
or a protected derivative thereof wherein one of E₂ₐ₁, E_{2b1}, E_{2c1} is —C(-L^{3a})= and the other two respectively are E₂ and E₃, one of L^{2a} and L^{3a} is —NH₂ and the other is —C(O)-A¹⁷-Y² or —C(O)-A¹⁷-Y³ as appropriate, and Y, E₁, E₂, E₃, E₄, D₁, D₂, D₃, L¹ and Y¹ are as defined in claim 1, with:
(A) when A¹⁶ is —C(O)N(R^{17b})—, in which R^{17b} is H:
(a) a compound of formula IV,
Y^{a}—N═C═O IV
; or
(b) with CO, a reagent that is a suitable source of CO, or phosgene or triphosgene in the presence of a compound of formula V,
Y^{a}—NH₂ V
wherein, in both cases, Y^{a} is Y² or Y³ as defined in claim 1;
(B) when A¹⁶ is a direct bond, with a compound of formula VI,
Y^{a}-L^{a} VI
wherein L^{a} is a suitable leaving group and Y^{a} is as defined above;
(C) when A¹⁶ is —S(O)₂—, —C(O)— or —C(O)—C(R^{y6})(R^{y7})—, with a compound of formula VII,
Y^{a}-A^{16a}-L^{a} VII
wherein A^{16a} is —S(O)₂—, —C(O)— or —C(O)—C(R^{y6})(R^{y7})—, and Y^{a} and L^{a} are as defined above;
(iii) for compounds of formula I in which one of L² and L³ is —C(O)-A¹⁷- and the other is —N(R^{17a})C(O)N(R^{17b})—, in which R^{17a} and R^{17b} are H, reaction of a compound of formula VIII, 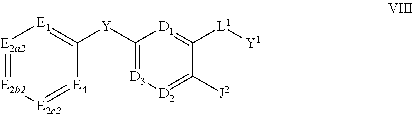
wherein one of E₂ₐ₂, E_{2b2}, E_{2c2} is —C(-J¹)= and the other two respectively are E₂ and E₃, one of J¹ and J² is —N═C═O and the other is —C(O)-A¹⁷-Y² or —C(O)-A¹⁷-Y³, and Y, E₁, E₂, E₃, E₄, D₁, D₂, D₃, L¹ and Y¹ are as defined in claim 1, with a compound of formula V as defined above;
(iv) reaction of a compound of formula IX, 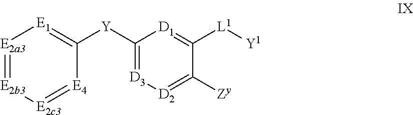
wherein one of E₂ₐ₃, E_{2b3}, E_{2c3} is —C(—Z^{x})═ and the other two respectively are E₂ and E₃, at least one of Z^{x} and Z^{y} is a suitable leaving group and the other may also is independently a suitable leaving group, or, Z^{y} is -L²-Y² and Z^{x} is -L³-Y³, and Y, E₁, E₂, E₃, E₄, D₁, D₂, D₃, L¹, Y¹, L², Y², L³ and Y³ are as defined in claim 1, with a compound of formula X,
Y^{a}-L^{x}-H X
wherein L^{x} is L² or L³ provided that at least one of L² and L³ is —C(O)A¹⁷-Y^{a}), and Y^{a} is as defined above;
(v) compounds of formula I in which there is a R^{17a} or R^{17b} group present that is not hydrogen, or if there is a R⁵, R⁶, R⁷, R⁸, R⁹, R¹¹, R¹², R¹³, R¹⁴, R¹⁶, R¹⁷ or R¹⁸ group present, which is attached to a heteroatom and which is not hydrogen, may be prepared by reaction of a corresponding compound of formula I in which such a group is present that is hydrogen with a compound of formula XI,
R^{wy}-L^{b} XI
wherein R^{wy} is either R^{17a} or R^{17b} as hereinbefore defined provided that it is not represent hydrogen, or R^{wy} is a R⁵ to R¹⁸ group in which those groups are not represent hydrogen, and L^{b} is a suitable leaving group;
(vi) for compounds of formula I that contain only saturated alkyl groups, reduction of a corresponding compound of formula I that contains an unsaturation, in the presence of suitable reducing conditions;
(vii) for compounds of formula I in which Y¹ is —C(O)OR^{9a}, in which R^{9a} is hydrogen or other carboxylic acid or ester protected derivatives, hydrolysis of a corresponding compound of formula I in which R^{9a} does not represent H;
(viii) for compounds of formula I in which Y¹ is —C(O)OR^{9a} and R^{9a} is not H:
(A) esterification of a corresponding compound of formula I in which R^{9a} is H; or
(B) trans-esterification of a corresponding compound of formula I in which R^{9a} does is not H and does not represent the same value of the corresponding R^{9a} group in the compound of formula I to be prepared,
in the presence of the appropriate alcohol of formula XII,
R^{9za}OH XII
in which R^{9za} is R^{9a} provided that it is not H;
(ix) for compounds of formula I in which Y¹ is —C(O)OR^{9a}, in which R^{9a} is other than H, and L¹ is as defined in claim 1, provided that it is not —(CH₂)ₚ-Q-(CH₂)_{q}— in which p is 0 and Q is —O—, reaction of a compound of formula XIII, 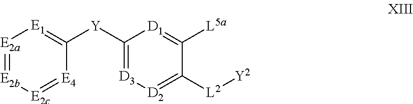
wherein L^{5a} is an appropriate alkali metal group, a —Mg-halide, a zinc-based group or a suitable leaving group, and Y, E₁, E₂ₐ, E_{2b}, E_{2c}, E₄, D₁, D₂, D₃, L² and Y² are as defined in claim 1, with a compound of formula XIV,
L⁶⁻L^{xy}-Y^{b} XIV
wherein L^{xy} is L¹, provided that it is not —(CH₂)ₚ-Q-(CH₂)_{q}— in which p is 0 and Q is —O—, and Y^{b} is —C(O)OR^{9a}, in which R^{9a} is other than H, and L⁶ is a suitable leaving group;
(x) for compounds of formula I in which L¹ is a single bond, and Y¹ is —C(O)OR^{9a} in which R^{9a} is H, reaction of a compound of formula XIII as defined above but in which L^{5a} is either:
(I) an alkali metal; or
(II) —Mg-halide,
with carbon dioxide, followed by acidification;
(xi) for compounds of formula I in which L¹ is a single bond, and Y¹ is —C(O)OR^{9a}, reaction of a corresponding compound of formula XIII as defined above but in which L^{5a} is a suitable leaving group, with CO or a reagent that is a suitable source of CO, in the presence of a compound of formula XV,
R^{9a}OH XV
wherein R^{9a} is as hereinbefore defined, and an appropriate catalyst system;
(xii) for compounds of formula I in which Y is —C(O)—, reaction of either a compound of formula XVI or XVII, 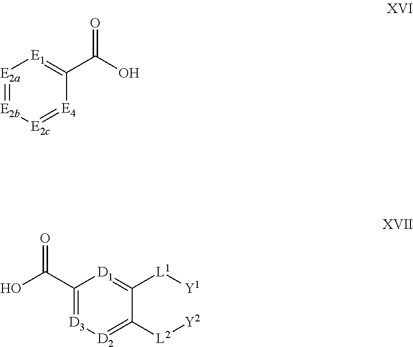
respectively with a compound of formula XVIII or XIX, 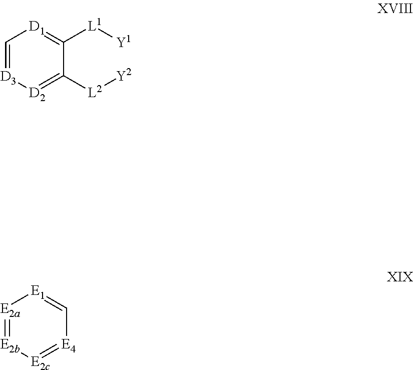
wherein E₁, E₂ₐ, E_{2b}, E_{2c}, E₄, D₁, D₂, D₃, L¹, Y¹, L² and Y² are as defined in claim 1;
(xiii) for compounds of formula I in which Y is —C(O)—, reaction of either a compound of formula XX or XXI, 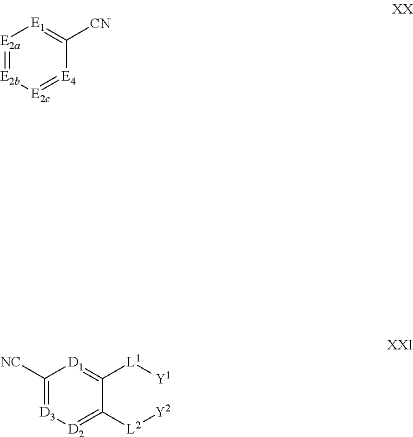
with a compound of formula XXII or XXIII, 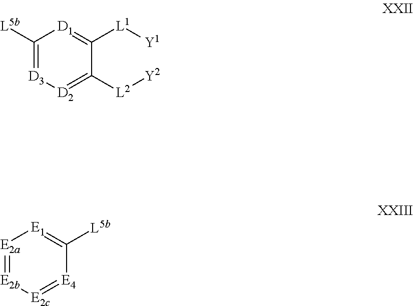
respectively, wherein L^{5b} is L^{5a} as defined above, and E₁, E₂ₐ, E_{2b}, E_{2c}, E₄, D₁, D₂, D₃, L¹, Y¹, L² and Y² are as defined in claim 1;
(xiv) for compounds of formula I in which Y is —C(O)—, reaction of an activated derivative of a compound of formula XVI or XVII as defined above, with a compound of formula XXII or XXIII as defined above, respectively;
(xvi) for compounds of formula I in which Y is —C(═N—OR²⁸)—, reaction of a corresponding compound of formula I in which Y is —C(O)—, with a compound of formula XXIIIA,
H₂N—O—R²⁸ XXIIIA
wherein R²⁸ is hydrogen or C₁₋₆ alkyl optionally substituted by one or more halo atoms;
(xvii) for compounds of formula I in which Y is —C(═N—OR²⁸)— and R²⁸ is C₁₋₆ alkyl optionally substituted by one or more halo atoms, reaction of a corresponding compound of formula I, in which R²⁸ is hydrogen, with a compound of formula XXIIIB,
R^{28a}-L⁷ XXIIIB
wherein R^{28a} is R²⁸, provided that it is not hydrogen and L⁷ is a suitable leaving group.

20. A process for the preparation of a pharmaceutical formulation as defined in claim 9, which process comprises bringing into association the compound of, or a pharmaceutically acceptable salt thereof with a pharmaceutically-acceptable adjuvant, diluent or carrier.

21. A process for the preparation of a combination product of claim 16, which process comprises bringing into association the compound, or a pharmaceutically acceptable salt thereof with the second therapeutic agent that is useful in the treatment of a respiratory disorder and/or inflammation, and at least one pharmaceutically-acceptable adjuvant, diluent or carrier.
